(19) United States
(12) Patent Application Publication
(10) Pub. No.: US 2026/0325466 A1
(43) Pub. Date: Oct. 22, 2026

(54) COPPER CHELATION THERAPY

(71) Applicants: NewSouth Innovations Pty Limited, Sydney (AU); Cure Cancer Australia Foundation, Sydney (AU)

(72) Inventors: Orazio VITTORIO, Sydney (AU); Jourdin ROUAEN, Sydney (AU)

(21) Appl. No.: 19/484,472

(22) PCT Filed: May 16, 2024
(86) PCT No.: PCT/AU2024/050490

§ 371 Date: Nov. 14, 2025

(30) Foreign Application Priority Data
May 16, 2023 (AU) 2023901504

(51) Int. Cl. A61K 31/133; A61K 31/4745; A61K 31/495; A61K 39/395; A61K 40/11; A61K 40/31; A61K 40/42; A61P 35/00

(52) U.S. Cl. CPC A61K 31/133; A61K 31/4745; A61K 31/495; A61K 39/3955; A61K 39/39558; A61K 40/11; A61K 40/31; A61K 40/4258; A61P 35/00

(57) ABSTRACT

Disclosed herein is the use of copper chelators, or agents that are capable of inducing copper chelators, for treating cancer, in particular for enhancing the efficiency of immunotherapies for treating cancer. Disclosed herein are methods for treating cancer, comprising administering a therapeutically effective amount of a copper chelator or a copper chelator inducing agent to a subject in need thereof in combination with a therapeutically effective amount of an anticancer immunotherapy agent. Also disclosed herein are kits or combinations and compositions comprising a copper chelator or a copper chelator inducing agent, and an anticancer immunotherapy agent, and uses thereof for treating cancer.

### CROSS REFERENCE

The present application claims priority to Australian provisional application no. 2023901504, the entire contents of which is incorporated by cross reference herein in its entirety.

### FIELD OF THE INVENTION

The invention relates to the use of copper chelators, or agents that are capable of inducing copper chelators, for treating cancer, in particular for enhancing the efficiency of immunotherapies for treating cancer. However, it will be appreciated that the invention is not limited to this particular field of use.

### BACKGROUND

The following discussion of the prior art is provided to place the invention in an appropriate technical context and enable the advantages of it to be more fully understood. It should be appreciated, however, that any discussion of the prior art throughout the specification should not be considered as an express or implied admission that such prior art is widely known or forms part of the common general knowledge in the field.

Despite great investment and advances, current treatments for cancer are still far from perfect. There is a need for further advances in therapeutic methods for treating different types of cancer.

Immunotherapy has improved the survival of cancer patients; however, efficacy of immunotherapy treatments can be hampered by the immunosuppressive tumor microenvironment (FIG. 1). For example, one mechanism that cancer cells use to protect themselves from antitumor immune responses is overexpression of programmed death ligand 1 (PD-L1). The immune checkpoint protein programmed death receptor 1 (PD-1) expressed by lymphocytes negatively regulates T-cell effector functions, leading to reduced cytokine production and cytotoxic activity against target cells, including tumor cells.

Several therapeutic mAbs targeting PD-L1/PD-1 have been approved by the FDA for adult melanoma and lung cancer. However, their efficacy, particularly for PD-L1/PD-1 blockade, is limited by cancer cell resistance in many patients and immune-related adverse events in others. There is a need to develop new treatments that can suppress the mechanisms by which cancer cells protect themselves from immune system attack in order to allow improvements in patient survival for immunotherapeutic oncological treatments. Such immunotherapeutic treatments include, for example, the use of immune checkpoint inhibitors, chimeric antigen receptor (CAR) T-cell therapy, the use of bispecific T-cell engagers (BiTEs), (CAR) NK-cell therapy, adoptive cell therapy and antibody-dependent cell cytotoxicity (ADCC) therapy.

It is an object of the present invention to overcome or ameliorate one or more the disadvantages of the prior art, or at least to provide a useful alternative.

### SUMMARY OF THE INVENTION

The inventors of the present application have surprisingly discovered that the combination of a copper chelator, or copper chelating inducing agent, with an anticancer immunotherapy agent provides a synergistic improvement in cancer treatment.

In a first aspect of the invention there is provided a method of treating cancer, comprising administering a therapeutically effective amount of a copper chelator or a copper chelator inducing agent to a subject in need thereof in combination with a therapeutically effective amount of an anticancer immunotherapy agent.

In a second aspect of the invention there is provided use of a copper chelator or a copper chelator inducing agent for the manufacture of a medicament for treating cancer in a subject in need thereof, wherein the copper chelator or copper chelator inducing agent is administered or is to be administered to the subject in combination with an anticancer immunotherapy agent.

In a third aspect of the invention there is provided use of an anticancer immunotherapy agent for the manufacture of a medicament for treating cancer in a subject in need thereof, wherein the anticancer immunotherapy agent is administered or is to be administered to the subject in combination with a copper chelator or a copper chelator inducing agent.

In a fourth aspect of the invention there is provided use of a copper chelator or a copper chelator inducing agent and an anticancer immunotherapy agent for the manufacture of a medicament for treating cancer in a subject in need thereof.

In a fifth aspect of the invention there is provided a copper chelator or a copper chelator inducing agent for use in the treatment of cancer in a subject in need thereof, wherein said copper chelator or copper chelator inducing agent is administered or is to be administered to the subject in combination with an anticancer immunotherapy agent.

In a sixth aspect of the invention there is provided an anticancer immunotherapy agent for use in the treatment of cancer in a subject in need thereof, wherein said anticancer immunotherapy agent is administered or is to be administered to the subject in combination with a copper chelator or a copper chelator inducing agent.

In a seventh aspect of the invention there is provided a kit or combination comprising a copper chelator or a copper chelator inducing agent, and an anticancer immunotherapy agent for use in the treatment of cancer in a subject in need thereof, optionally the kit or combination may further include instructions for administration of the copper chelator or copper chelator inducing agent, and for administration of the anticancer immunotherapy agent for the treatment of cancer.

The following options may be used in conjunction with the first, second, third, fourth, fifth, sixth, or seventh aspect, either individually or in any combination.

In certain embodiments, the copper chelator or copper chelator inducing agent and anticancer agent are administered, or are to be administered, separately to the subject.

In certain embodiments, the copper chelator or copper chelator inducing agent and anticancer agent are administered, or are to be administered, concurrently to the subject.

In certain embodiments, the anticancer immunotherapy agent is selected from the group consisting of antibodies, immune checkpoint inhibitors (e.g. α-CTLA4 targeting ICI and/or α-PDL1 targeting ICI), chimeric antigen receptor (CAR) T-cells, bispecific T-cell engagers (BiTEs), (CAR) NK-cells, adoptive immune cells and antibody-dependent immune cells. In certain specific embodiments, the anticancer immunotherapy agent is an anti-GD2 immunotherapy agent. In certain specific embodiments, the anticancer immunotherapy agent is a GD2-directed chimeric antigen receptor (CAR) T cell or an anti-GD2 antibody. In certain specific embodiments, the anticancer immunotherapy agent is an anti-GD2 antibody. In certain specific embodiments, the anticancer immunotherapy agent is selected from the group consisting of: atezolizumab (Tecentriq), avelumab (Bavencio), dostarlizumab (Jemperli), durvalumab (Imfinzi), ipilimumab (Yervoy), nivolumab (Opdivo), dinutiximab (Unituxin), dinutuxinab beta (Qarziba) and pembrolizumab (Keytruda).

In certain embodiments, the subject is selected from the group consisting of humans, pets and livestock. In a specific embodiment, the subject is a human.

A skilled person will appreciate that all tumours utilize copper, and accordingly the inventive treatment would be suitable for treating all types of cancer. In one embodiment the cancer is selected from the group consisting of lung cancer; breast cancer; colorectal cancer; anal cancer; pancreatic cancer; eye cancer; prostate cancer; ovarian carcinoma; liver and bile duct carcinoma; esophageal carcinoma; non-Hodgkin's lymphoma; bladder carcinoma; carcinoma of the uterus; glioma, glioblastoma, medulloblastoma, and other tumors of the brain; kidney cancer; myelofibrosis, cancer of the head and neck; cancer of the stomach; multiple myeloma; testicular cancer; germ cell tumor; neuroendocrine tumor; cervical cancer; oral cancer; carcinoids of the gastrointestinal tract, breast, and other organs; signet ring cell carcinoma; mesenchymal tumors including sarcomas, fibrosarcomas, haemangioma, angiomatosis, haemangiopericytoma, pseudoangiomatous stromal hyperplasia, myofibroblastoma, fibromatosis, inflammatory myofibroblastic tumour, lipoma, angiolipoma, granular cell tumour, neurofibroma, schwannoma, angiosarcoma, liposarcoma, rhabdomyosarcoma, osteosarcoma, leiomyoma or a leiomysarcoma. In a further embodiment, the cancer is selected from the group consisting of hepatocellular carcinoma, adenocarcinoma, breast cancer and prostate cancer. In a further embodiment, the cancer is selected from the group consisting of neuroblastoma, breast cancer, glioblastoma, ovarian cancer, lung cancer, prostate cancer, stomach cancer, mesothelioma, liver cancer, cervical cancer, bladder cancer, thyroid cancer, pancreatic cancer, oral cancer, osteosarcoma, head and neck cancers, medulloblastoma, lower-grade gliomas, DMG (diffuse midline glioma), DIPG (diffuse intrinsic pontine glioma), and leukemia. In one embodiment the cancer is breast cancer or neuroblastoma. In one embodiment, the cancer is selected from neuroblastoma and mesothelioma. In one embodiment the cancer is neuroblastoma. In one embodiment, the cancer is treatable by modulation of GD2, by for example, treatment with an anti-GD2 immunotherapy agent, such as an anti-GD2 antibody or a GD2-directed chimeric antigen receptor (CAR) T cell. In certain embodiments, the cancer expresses GD2. In certain embodiments, the cancer is MYCN-mediated.

In one embodiment lung cancer includes lung adenocarcinoma, squamous cell carcinoma, large cell carcinoma, bronchoalveolar carcinoma, non-small-cell carcinoma, small cell carcinoma and mesothelioma. In one embodiment breast cancer includes ductal carcinoma, lobular carcinoma, inflammatory breast cancer, clear cell carcinoma, and mucinous carcinoma. In one embodiment colorectal cancer includes colon cancer and rectal cancer. In one embodiment pancreatic cancer includes pancreatic adenocarcinoma, islet cell carcinoma and neuroendocrine tumors.

In one embodiment ovarian carcinoma includes ovarian epithelial carcinoma or surface epithelial-stromal tumour including serous tumour, endometrioid tumor and mucinous cystadenocarcinoma, and sex-cord-stromal tumor. In one embodiment liver and bile duct carcinoma includes hepatocellular carcinoma, cholangiocarcinoma and hemangioma. In one embodiment esophageal carcinoma includes esophageal adenocarcinoma and squamous cell carcinoma. In one embodiment carcinoma of the uterus includes endometrial adenocarcinoma, uterine papillary serous carcinoma, uterine clear-cell carcinoma, uterine sarcomas and leiomyosarcomas and mixed Mullerian tumors. In one embodiment kidney cancer includes renal cell carcinoma, clear cell carcinoma and Wilm's tumor. In one embodiment cancer of the head and neck includes squamous cell carcinomas. In one embodiment cancer of the stomach includes stomach adenocarcinoma and gastrointestinal stromal tumor. In one embodiment eye cancer include retinoblastoma and uveal melanoma.

In certain embodiments, the copper chelator is selected from the group consisting of: ethylenediamine, polyethylenimine and derivatives thereof, tetrathiomolybdate; N,N′-bis(2-aminoethyl)ethane-1,2-diamine (trientine, triethylenetetramine, TETA); penicillamine; D-penicillamine; N′-(2-aminoethyl)-N-[2-(2-amino ethylamino) ethyl]ethane-1,2-diamine (tetraethylenepentamine, TEPA); 2-[2-[bis(carboxymethyl)amino]ethyl-(carboxymethyl)amino]acetic acid (EDTA); 2-[bis[2-[bis(carboxymethyl)amino]ethyl] amino]acetic acid (DTPA); N,N,N′,N′-tetrakis(2-pyridyl-methyl) ethylenediamine (TPEN); 2-[4,8,11-tris(carboxymethyl)-1,4,8,11-tetrazacyclotetradec-1-yl]acetic acid; 2-[4,7,10-tris(carboxymethyl)-1,4,7,10-tetrazacyclododec-1-yl]acetic acid (DOTA); (2S)-2-amino-3-methyl-3-sulfanylbutanoic acid (D-pen); bis(sulfanylidene)molybdenum sulfanide (TTM); 5-chloro-7-iodo-8-hydroxy-quinoline (Cliquinol); metformin; diethylcarbamodithioic acid (DDC); 2,9-dimethyl-1,10-phenanthroline (Neocuproine); 2,9-dimethyl-4,7-diphenyl-1,10-phenanthroline (Bathocuproine); 4-[2,9-dimethyl-7-(4-sulfophenyl)-1,10-phenanthrolin-4-yl]benzenesulfonic acid (BCS); and N,N′-bis(cyclohexylideneamino)oxamide (Cuprizone). In certain embodiments, the copper chelator is selected from the group consisting of triethylenetetramine, tetraethylenepentamine, penicillamine, tetrathiomolibdic acid, and salts thereof. In certain specific embodiments, the copper chelator is selected from the group consisting of tetraethylenepentamine, triethylenetetramine, and salts thereof. In certain specific embodiments, the copper chelator is triethylenetetramine, or a salt thereof.

In certain embodiments, the copper chelator inducing agent is a zinc salt.

In certain specific embodiments, the copper chelator is TEPA or TETA, and the anticancer immunotherapy agent is an anti-GD2 immunotherapy agent, optionally the anticancer immunotherapy agent is a GD2-directed chimeric antigen receptor (CAR) T cell or an anti-GD2 antibody, optionally the anticancer immunotherapy agent is anti-GD2 mAb.

In certain embodiments, the copper chelator is trientine tetrahydrochloride and the anticancer immunotherapy comprises dinutuximab-beta, optionally the trientine tetrahydrochloride and dinutuximab-beta is administered in combination with irinotecan and temozolamide, optionally wherein the cancer is neuroblastoma, optionally wherein the subject to be treated is a child (being aged less than 21, less than 18, or less than 17, 16, 15, 14, 13, or 12 years old).

In certain embodiments, the copper chelator or copper chelator inducing agent is dosed through a different route to the anticancer immunotherapy agent. For example, in certain embodiments, the copper chelator or copper inducing agent is dosed orally, and the anticancer immunotherapy is dosed parenterally, optionally intra peritoneally.

In certain specific embodiments, the copper chelator is TEPA or TETA dosed orally, and the anticancer immunotherapy agent is an anti-GD2 immunotherapy agent, such as a GD2-directed chimeric antigen receptor (CAR) T cell or an anti-GD2 antibody, dosed parenterally, optionally intra peritoneally. In certain specific embodiments, the copper chelator is TEPA or TETA dosed orally, and the anticancer immunotherapy agent is anti-GD2 mAb dosed parenterally, optionally intra peritoneally.

In certain embodiments, the copper chelator or copper chelator inducing agent is dosed every day, every second day, every third day, weekly, or every fortnightly.

In certain embodiments, the immunotherapy agent is dosed every day, every second day, every third day, weekly, or every fortnightly.

In certain specific embodiments, the copper chelator is TEPA or TETA dosed orally for at least three, four or five or more days per week, and the anticancer immunotherapy agent is anti-GD2 mAb dosed parenterally, optionally intra peritoneally, at least one, two, or three or more days per week.

In certain specific embodiments, the copper chelator is TEPA or TETA dosed orally for at least three, four or five or more days per week, and the anticancer immunotherapy agent is an anti-GD2 immunotherapy agent, optionally a GD2-directed chimeric antigen receptor (CAR) T cell or an anti-GD2 antibody, dosed parenterally, optionally intra peritoneally, at least one, two, or three or more days per week.

In certain embodiments, the dosage of the copper chelator or copper chelator inducing agent at each administration may be from about 0.5 mg/kg to about 4000 mg/kg, or from about 1 mg/kg to about 3000 mg/kg, about 1 mg/kg to about 2000 mg/kg, about 1 mg/kg to about 950 mg/kg, about 1 mg/kg to about 950 mg/kg, about 10 mg/kg to about 950 mg/kg, about 21 mg/kg to about 950 mg/kg, about 41 mg/kg to about 950 mg/kg, about 60 mg/kg to about 950 mg/kg, about 80 mg/kg to about 950 mg/kg, about 100 mg/kg to about 950 mg/kg, about 150 mg/kg to about 950 mg/kg, about 200 mg/kg to about 950 mg/kg, about 250 mg/kg to about 950 mg/kg, about 300 mg/kg to about 950 mg/kg, about 350 mg/kg to about 950 mg/kg, about 400 mg/kg to about 950 mg/kg, about 450 mg/kg to about 950 mg/kg, about 500 mg/kg to about 950 mg/kg, about 550 mg/kg to about 950 mg/kg, about 600 mg/kg to about 950 mg/kg, about 1 mg/kg to about 920 mg/kg, about 1 mg/kg to about 880 mg/kg, about 1 mg/kg to about 840 mg/kg, about 1 mg/kg to about 810 mg/kg, about 1 mg/kg to about 780 mg/kg, about 1 mg/kg to about 740 mg/kg, about 1 mg/kg to about 700 mg/kg, about 1 mg/kg to about 670 mg/kg, about 1 mg/kg to about 640 mg/kg, about 1 mg/kg to about 600 mg/kg, about 100 mg/kg to about 600 mg/kg, about 200 mg/kg to about 600 mg/kg, about 300 mg/kg to about 600 mg/kg, about 400 mg/kg to about 600 mg/kg, about 500 mg/kg to about 600 mg/kg, about 100 mg/kg to about 500 mg/kg, about 100 mg/kg to about 400 mg/kg, about 100 mg/kg to about 300 mg/kg, or about 100 mg/kg to about 200 mg/kg. It may be greater than or equal to about 1 mg/kg, 11 mg/kg, 21 mg/kg, 31 mg/kg, 41 mg/kg, 50 mg/kg, 60 mg/kg, 70 mg/kg, 80 mg/kg, 90 mg/kg, 100 mg/kg, 150 mg/kg, 200 mg/kg, 250 mg/kg, or 300 mg/kg. It may be less than or equal to about 4000 mg/kg, 3000 mg/kg, 2000 mg/kg, 1000 mg/kg, 950 mg/kg, 920 mg/kg, 880 mg/kg, 840 mg/kg, 810 mg/kg, 780 mg/kg, 740 mg/kg, 700 mg/kg, 670 mg/kg, 640 mg/kg, 600 mg/kg, 550 mg/kg, 500 mg/kg, 450 mg/kg, or 400 mg/kg. In certain embodiments, it may be, for example, about 1 mg/kg, 11 mg/kg, 21 mg/kg, 31 mg/kg, 41 mg/kg, 50 mg/kg, 60 mg/kg, 70 mg/kg, 80 mg/kg, 90 mg/kg, 100 mg/kg, 120 mg/kg, 150 mg/kg, 180 mg/kg, 200 mg/kg, 220 mg/kg, 250 mg/kg, 280 mg/kg, 300 mg/kg, 320 mg/kg, 350 mg/kg, 380 mg/kg, 400 mg/kg, 420 mg/kg, 450 mg/kg, 480 mg/kg, 500 mg/kg, 520 mg/kg, 550 mg/kg, 580 mg/kg, 600 mg/kg, 670 mg/kg, 700 mg/kg, 740 mg/kg, 780 mg/kg, 810 mg/kg, 840 mg/kg, 880 mg/kg, 920 mg/kg, 950 mg/kg, 1000 mg/kg, 1100 mg/kg, 1200 mg/kg, 1500 mg/kg, 2000 mg/kg, 2500 mg/kg, 3000 mg/kg, 3500 mg/kg, or 4000 mg/kg.

In certain embodiments, the dosage of the anticancer immunotherapy agent at each administration may be from about 0.01 mg/m² (where m² is the body surface area of the subject) to about 250 mg/m², or from about 4 mg/m² to about 250 mg/m², about 8 mg/m² to about 250 mg/m², about 12 mg/m² to about 250 mg/m², about 16 mg/m² to about 250 mg/m², about 20 mg/m² to about 250 mg/m², about 23 mg/m² to about 250 mg/m², about 26 mg/m² to about 250 mg/m², about 29 mg/m² to about 250 mg/m², about 32 mg/m² to about 250 mg/m², about 35 mg/m² to about 250 mg/m², about 38 mg/m² to about 250 mg/m², about 41 mg/m² to about 250 mg/m², about 44 mg/m² to about 250 mg/m², about 47 mg/m² to about 250 mg/m², about 50 mg/m² to about 250 mg/m², about 0.01 mg/m² to about 230 mg/m², about 0.01 mg/m² to about 210 mg/m², about 0.01 mg/m² to about 190 mg/m², about 0.01 mg/m² to about 170 mg/m², about 0.01 mg/m² to about 150 mg/m², about 0.01 mg/m² to about 130 mg/m², about 0.01 mg/m² to about 110 mg/m², about 0.01 mg/m² to about 90 mg/m², about 0.01 mg/m² to about 70 mg/m², about 0.01 mg/m² to about 50 mg/m², about 20 mg/m² to about 50 mg/m², about 26 mg/m² to about 50 mg/m², about 32 mg/m² to about 50 mg/m², about 38 mg/m² to about 50 mg/m², about 44 mg/m² to about 50 mg/m², about 20 mg/m² to about 44 mg/m², about 20 mg/m² to about 38 mg/m², about 20 mg/m² to about 32 mg/m², or about 20 mg/m² to about 26 mg/m². It may be greater than or equal to about 0.01 mg/m², 2 mg/m², 4 mg/m², 6 mg/m², 8 mg/m², 10 mg/m², 12 mg/m², 14 mg/m², 16 mg/m², 18 mg/m², 20 mg/m², 23 mg/m², 26 mg/m², 29 mg/m², or 32 mg/m². It may be less than or equal to about 250 mg/m², 230 mg/m², 210 mg/m², 190 mg/m², 170 mg/m², 150 mg/m², 130 mg/m², 110 mg/m², 90 mg/m², 70 mg/m², 50 mg/m², 47 mg/m², 44 mg/m², 41 mg/m², or 38 mg/m². In certain embodiments, it may be, for example, about 0.01 mg/m², 2 mg/m², 4 mg/m², 6 mg/m², 8 mg/m², 10 mg/m², 12 mg/m², 14 mg/m², 16 mg/m², 18 mg/m², 20 mg/m², 22 mg/m², 23 mg/m², 24 mg/m², 26 mg/m², 28 mg/m², 29 mg/m², 30 mg/m², 32 mg/m², 34 mg/m², 35 mg/m², 36 mg/m², 38 mg/m², 40 mg/m², 41 mg/m², 42 mg/m², 44 mg/m², 46 mg/m², 47 mg/m², 48 mg/m², 50 mg/m², 90 mg/m², 110 mg/m², 130 mg/m², 150 mg/m², 170 mg/m², 190 mg/m², 210 mg/m², 230 mg/m², or 250 mg/m².

In certain embodiments, the copper chelator, copper chelator inducing agent and/or the anticancer immunotherapy agent may individually or together (i.e., the copper chelator and the anticancer immunotherapy agent; or alternatively the copper chelator inducing agent and the anticancer immunotherapy agent) be in the form of a pharmaceutical composition.

### Pharmaceutical Composition

In one embodiment there is provided a pharmaceutical composition, which may comprise the copper chelator as described hereinbefore, the copper chelator inducing agent as described hereinbefore, the anticancer immunotherapy agent as described hereinbefore, the copper chelator as described hereinbefore and the anticancer immunotherapy agent as described hereinbefore, or the copper chelator inducing agent as described hereinbefore and the anticancer immunotherapy agent as described hereinbefore; and a pharmaceutically acceptable excipient.

The phrase “pharmaceutically acceptable excipient” includes pharmaceutically acceptable carriers and diluents. The phrase “pharmaceutically acceptable carrier” refers to any carrier known to those skilled in the art to be suitable for the particular mode of administration. In addition, the components described herein before may be formulated as the sole pharmaceutically active ingredient in the composition or may be combined with other active ingredients.

The phrase “pharmaceutically acceptable salt” refers to any salt preparation that is appropriate for use in a pharmaceutical application. By pharmaceutically acceptable salt it is meant those salts which, within the scope of sound medical judgement, are suitable for use in contact with the tissues of humans and lower animals without undue toxicity, irritation, allergic response and the like, and are commensurate with a reasonable benefit/risk ratio. Pharmaceutically acceptable salts are well known in the art and include acid addition and base salts. Hemisalts of acids and bases may also be formed. Pharmaceutically acceptable salts include salts of mineral acids (e.g., hydrochlorides, hydrobromides, sulfates, and the like); and salts of organic acids (e.g., formates, acetates, lactates, malates, tartrates, citrates, ascorbates, succinates, maleates, butyrates, valerates, fumarates, and the like).

The components of the pharmaceutical composition (i.e. the copper chelator as described hereinbefore, the copper chelator inducing agent as described hereinbefore, the anticancer immunotherapy agent as described hereinbefore, the copper chelator as described hereinbefore and the anticancer immunotherapy agent as described hereinbefore, or the copper chelator inducing agent as described hereinbefore and the anticancer immunotherapy agent as described hereinbefore) may exist in both unsolvated and solvated forms. The term ‘solvate’ is used herein to describe a molecular complex comprising the copper chelator, copper chelator inducing agent, and/or anticancer immunotherapy agent and a stoichiometric amount of one or more pharmaceutically acceptable solvent molecules, for example, ethanol. The term ‘hydrate’ is employed when the solvent is water.

In one embodiment the copper chelator, copper chelator inducing agent, and/or anticancer immunotherapy agent described herein may be administered in the form of a “prodrug”. The phrase “prodrug” refers to a compound that, upon in vivo administration, is metabolized by one or more steps or processes or otherwise converted to the biologically, pharmaceutically or therapeutically active form of the copper chelator, copper chelator inducing agent, and/or anticancer immunotherapy agent described hereinbefore. Prodrugs can be prepared by modifying functional groups present in the compound in such a way that the modifications are cleaved, either in routine manipulation or in vivo, to a copper chelator, copper chelator inducing agent, and/or anticancer immunotherapy agent described herein.

Compositions herein comprise one or more copper chelators, copper chelator inducing agents, and/or anticancer immunotherapy agents provided herein. The copper chelators, copper chelator inducing agents, and/or anticancer immunotherapy agents are, in one embodiment, formulated into suitable pharmaceutical preparations such as solutions, suspensions, tablets, creams, gels, dispersible tablets, pills, capsules, powders, sustained release formulations or elixirs, for oral administration or in sterile solutions or suspensions for parenteral administration, as well as transdermal patch preparation and dry powder inhalers. In one embodiment, the copper chelators, copper chelator inducing agents, and/or anticancer immunotherapy agents described above are formulated into pharmaceutical compositions using techniques and procedures well known in the art (see, e.g., Ansel Introduction to Pharmaceutical Dosage Forms, Fourth Edition 1985, 126).

In the compositions, effective concentrations of one or more copper chelators, copper chelator inducing agents, and/or anticancer immunotherapy agents or pharmaceutically acceptable derivatives thereof is (are) mixed with a suitable pharmaceutical carrier. The copper chelators, copper chelator inducing agents, and/or anticancer immunotherapy agents may be derivatized as the corresponding salts, esters, acids, bases, solvates, hydrates or prodrugs prior to formulation, as described above. The concentrations of the copper chelators, copper chelator inducing agents, and/or anticancer immunotherapy agents in the compositions are effective for delivery of an amount, upon administration, that treats, prevents, or ameliorates one or more of the symptoms of diseases or disorders to be treated.

In one embodiment, the compositions are formulated for single dosage administration. To formulate a composition, the weight fraction of copper chelator, copper chelator inducing agent, and/or anticancer immunotherapy agent is dissolved, suspended, dispersed or otherwise mixed in a selected carrier at an effective concentration such that the treated condition is relieved, prevented, or one or more symptoms are ameliorated.

The copper chelator, copper chelator inducing agent, and/or anticancer immunotherapy agent is included in the pharmaceutically acceptable carrier in an amount sufficient to exert a therapeutically useful effect in the absence of undesirable side effects on the patient treated. The therapeutically effective concentration may be determined empirically by testing the copper chelator, copper chelator inducing agent, and/or anticancer immunotherapy agent in in vitro and in vivo systems described herein, and then extrapolated from there for dosages for humans.

The concentration of copper chelator, copper chelator inducing agent, and/or anticancer immunotherapy agent in the pharmaceutical composition will depend on absorption, distribution, inactivation and excretion rates of the copper chelator, copper chelator inducing agent, and/or anticancer immunotherapy agent, the physicochemical characteristics of the copper chelator, copper chelator inducing agent, and/or anticancer immunotherapy agent, the dosage schedule, and amount administered as well as other factors known to those of skill in the art.

In one embodiment, a therapeutically effective dosage should produce a serum concentration of active ingredient (i.e., copper chelator, copper chelator inducing agent, and/or anticancer immunotherapy agent) of from about 0.1 ng/mL to about 50-100 μg/mL. The pharmaceutical compositions, in another embodiment, should provide a dosage of from about 0.001 mg to about 2000 mg of copper chelator, copper chelator inducing agent, and/or anticancer immunotherapy agent per kilogram of body weight per day. Pharmaceutical dosage unit forms are prepared to provide from about 0.01 mg, 0.1 mg or 1 mg to about 500 mg, 1000 mg or 2000 mg, and in one embodiment from about 10 mg to about 500 mg of the active ingredient or a combination of essential ingredients per dosage unit form.

Dosing may occur at intervals of minutes, hours, days, weeks, months or years or continuously over any one of these periods. Suitable dosages lie within the range of about 0.1 ng per kg of body weight to 1 g per kg of body weight per dosage. The dosage is preferably in the range of 1 μg to 1 g per kg of body weight per dosage, such as is in the range of 1 mg to 1 g per kg of body weight per dosage. Suitably, the dosage is in the range of 1 μg to 500 mg per kg of body weight per dosage, such as 1 μg to 200 mg per kg of body weight per dosage, or 1 μg to 100 mg per kg of body weight per dosage. Other suitable dosages may be in the range of 1 mg to 250 mg per kg of body weight, including 1 mg to 10, 20, 50 or 100 mg per kg of body weight per dosage or 10 μg to 100 mg per kg of body weight per dosage.

Suitable dosage amounts and dosing regimens can be determined by the attending physician and may depend on the particular condition being treated, the severity of the condition, as well as the general health, age and weight of the subject.

In instances in which the copper chelator, copper chelator inducing agent, and/or anticancer immunotherapy agent exhibit insufficient solubility, methods for solubilizing such substances may be used. Such methods are known to those of skill in this art, and include, but are not limited to, using cosolvents, such as dimethyl sulfoxide (DMSO), using surfactants, such as TWEEN®, dissolution in aqueous sodium bicarbonate, formulating the substances of interest as nanoparticles, and the like. Derivatives of the substances, such as prodrugs of the substances may also be used in formulating effective pharmaceutical compositions.

Upon mixing or addition of the copper chelator, copper chelator inducing agent, and/or anticancer immunotherapy agent, the resulting mixture may be a solution, suspension, emulsion or the like. The form of the resulting mixture depends upon a number of factors, including the intended mode of administration and the solubility of the copper chelator, copper chelator inducing agent, and/or anticancer immunotherapy agent in the selected carrier or vehicle. The effective concentration is sufficient for ameliorating the symptoms of the disease, disorder or condition treated and may be empirically determined.

The pharmaceutical compositions are provided for administration to humans and animals in unit dosage forms, such as tablets, capsules, pills, powders, granules, sterile parenteral solutions or suspensions, and oral solutions or suspensions, and oil-water emulsions containing suitable quantities of the copper chelator, copper chelator inducing agent, and/or anticancer immunotherapy agent or pharmaceutically acceptable derivatives thereof. The pharmaceutically therapeutically active substances and derivatives thereof are, in one embodiment, formulated and administered in unit-dosage forms or multiple-dosage forms. The active ingredient may be administered at once or may be divided into a number of smaller doses to be administered at intervals of time. Unit-dose forms as used herein refer to physically discrete units suitable for human and animal subjects and packaged individually as is known in the art. Each unit-dose contains a predetermined quantity of the therapeutically active copper chelator, copper chelator inducing agent, and/or anticancer immunotherapy agent sufficient to produce the desired therapeutic effect, in association with the required pharmaceutical carrier, vehicle or diluent. Examples of unit-dose forms include ampules and syringes and individually packaged tablets or capsules. Unit-dose forms may be administered in fractions or multiples thereof. A multiple-dose form is a plurality of identical unit-dosage forms packaged in a single container to be administered in segregated unit-dose form. Examples of multiple-dose forms include vials, bottles of tablets or capsules or bottles of pints or gallons. Hence, multiple dose form is a multiple of unit-doses which are not segregated in packaging.

Actual methods of preparing such dosage forms are known, or will be apparent, to those skilled in this art; for example, see Remington's Pharmaceutical Sciences, Mack Publishing Company, Easton, Pa., 15th Edition, 1975.

Dosage forms or compositions containing active ingredient in the range of 0.005% to 100% (wt. %) with the balance made up from non-toxic carrier may be prepared. Methods for preparation of these compositions are known to those skilled in the art. The contemplated compositions may contain 0.001%-100% (wt. %) active ingredient, in one embodiment 0.1-95% (wt. %), in another embodiment 75-85% (wt. %).

### Modes of Administration

Convenient modes of administration include injection (subcutaneous, intravenous, intraperitoneal etc.), oral administration, inhalation, transdermal application, topical creams or gels or powders, ocular, optic and nasal dosage forms, vaginal or rectal administration. Depending on the route of administration, the formulation and/or compound (i.e., copper chelator, copper chelator inducing agent, and/or anticancer immunotherapy agent) may be coated with a material to protect the compound from the action of enzymes, acids and other natural conditions which may inactivate the therapeutic activity of the compound. The compound may also be administered parenterally or intraperitoneally.

### Compositions for Oral Administration

Oral pharmaceutical dosage forms are either solid, gel or liquid. The solid dosage forms are tablets, capsules, granules, and bulk powders. Types of oral tablets include compressed, chewable lozenges and tablets which may be enteric-coated, sugar-coated, sustained release or film-coated. Capsules may be hard or soft gelatin capsules, while granules and powders may be provided in non-effervescent or effervescent form with the combination of other ingredients known to those skilled in the art.

### Solid Compositions for Oral Administration

In certain embodiments, the formulations are solid dosage forms, in one embodiment, capsules or tablets. The tablets, pills, capsules, troches and the like can contain one or more of the following ingredients, or compounds of a similar nature: a binder; a lubricant; a diluent; a glidant; a disintegrating agent; a coloring agent; a sweetening agent; a flavoring agent; a wetting agent; an emetic coating; and a film coating. Examples of binders include microcrystalline cellulose, gum tragacanth, glucose solution, acacia mucilage, gelatin solution, molasses, polyvinylpyrrolidone, povidone, crospovidones, sucrose and starch paste. Lubricants include talc, starch, magnesium or calcium stearate, lycopodium and stearic acid. Diluents include, for example, lactose, sucrose, starch, kaolin, salt, mannitol and dicalcium phosphate. Glidants include, but are not limited to, colloidal silicon dioxide. Disintegrating agents include crosscarmellose sodium, sodium starch glycolate, alginic acid, corn starch, potato starch, bentonite, methylcellulose, agar and carboxymethylcellulose. Coloring agents include, for example, any of the approved certified water-soluble FD and C dyes, mixtures thereof; and water insoluble FD and C dyes suspended on alumina hydrate. Sweetening agents include sucrose, lactose, mannitol and artificial sweetening agents such as saccharin, and any number of spray dried flavors. Flavoring agents include natural flavors extracted from plants such as fruits and synthetic blends of compounds which produce a pleasant sensation, such as, but not limited to peppermint and methyl salicylate. Wetting agents include propylene glycol monostearate, sorbitan monooleate, diethylene glycol monolaurate and polyoxyethylene lauryl ether. Emetic-coatings include fatty acids, fats, waxes, shellac, ammoniated shellac and cellulose acetate phthalates. Film coatings include hydroxyethylcellulose, sodium carboxymethylcellulose, polyethylene glycol 4000 and cellulose acetate phthalate.

The copper chelator, copper chelator inducing agent, and/or anticancer immunotherapy agent, or pharmaceutically acceptable derivative thereof, could be provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

When the dosage unit form is a capsule, it can contain, in addition to material of the above type, a liquid carrier such as a fatty oil. In addition, dosage unit forms can contain various other materials which modify the physical form of the dosage unit, for example, coatings of sugar and other enteric agents. The copper chelator, copper chelator inducing agent, and/or anticancer immunotherapy agent can also be administered as a component of an elixir, suspension, syrup, wafer, sprinkle, chewing gum or the like. A syrup may contain, in addition to the active compounds, sucrose as a sweetening agent and certain preservatives, dyes and colorings and flavors.

The active materials can also be mixed with other active materials which do not impair the desired action, or with materials that supplement the desired action, such as antacids, H2 blockers, and diuretics. The active ingredient is a copper chelator, copper chelator inducing agent, and/or anticancer immunotherapy agent or pharmaceutically acceptable derivative thereof as described herein. Higher concentrations, up to about 98% by weight of the active ingredient may be included.

In certain embodiments, tablets and capsules formulations may be coated as known by those of skill in the art in order to modify or sustain dissolution of the active ingredient. Thus, for example, they may be coated with a conventional enterically digestible coating, such as phenylsalicylate, waxes and cellulose acetate phthalate.

### Liquid Compositions for Oral Administration

Liquid oral dosage forms include aqueous solutions, emulsions, suspensions, solutions and/or suspensions reconstituted from non-effervescent granules and effervescent preparations reconstituted from effervescent granules. Aqueous solutions include, for example, elixirs and syrups. Emulsions are either oil-in-water or water-in-oil.

Liquid pharmaceutically administrable compositions can, for example, be prepared by dissolving, dispersing, or otherwise mixing an active compound (i.e. copper chelator, copper chelator inducing agent, and/or anticancer immunotherapy agent) as defined above and optional pharmaceutical adjuvants in a carrier, such as, for example, water, saline, aqueous dextrose, glycerol, glycols, ethanol, and the like, to thereby form a solution or suspension. If desired, the pharmaceutical composition to be administered may also contain minor amounts of nontoxic auxiliary substances such as wetting agents, emulsifying agents, solubilizing agents, pH buffering agents and the like, for example, acetate, sodium citrate, cyclodextrin derivatives, sorbitan monolaurate, triethanolamine sodium acetate, triethanolamine oleate, and other such agents.

Elixirs are clear, sweetened, hydroalcoholic preparations. Pharmaceutically acceptable carriers used in elixirs include solvents. Syrups are concentrated aqueous solutions of a sugar, for example, sucrose, and may contain a preservative. An emulsion is a two-phase system in which one liquid is dispersed in the form of small globules throughout another liquid. Pharmaceutically acceptable carriers used in emulsions are non-aqueous liquids, emulsifying agents and preservatives. Suspensions use pharmaceutically acceptable suspending agents and preservatives. Pharmaceutically acceptable substances used in non-effervescent granules, to be reconstituted into a liquid oral dosage form, include diluents, sweeteners and wetting agents. Pharmaceutically acceptable substances used in effervescent granules, to be reconstituted into a liquid oral dosage form, include organic acids and a source of carbon dioxide. Coloring and flavoring agents are used in all of the above dosage forms.

Solvents include glycerin, sorbitol, ethyl alcohol and syrup. Examples of preservatives include glycerin, methyl and propylparaben, benzoic acid, sodium benzoate and ethanol. Examples of non-aqueous liquids utilized in emulsions include mineral oil and cottonseed oil. Examples of emulsifying agents include gelatin, acacia, tragacanth, bentonite, and surfactants such as polyoxyethylene sorbitan monooleate. Suspending agents include sodium carboxymethylcellulose, pectin, tragacanth, Veegum and acacia. Sweetening agents include sucrose, syrups, glycerin and artificial sweetening agents such as saccharin. Wetting agents include propylene glycol monostearate, sorbitan monooleate, diethylene glycol monolaurate and polyoxyethylene lauryl ether. Organic acids include citric and tartaric acid. Sources of carbon dioxide include sodium bicarbonate and sodium carbonate. Coloring agents include any of the approved certified water-soluble FD and C dyes, and mixtures thereof. Flavoring agents include natural flavors extracted from plants such fruits, and synthetic blends of compounds which produce a pleasant taste sensation.

For a solid dosage form, the solution or suspension, in for example propylene carbonate, vegetable oils or triglycerides, is in one embodiment encapsulated in a gelatin capsule. For a liquid dosage form, the solution, e.g., for example, in a polyethylene glycol, may be diluted with a sufficient quantity of a pharmaceutically acceptable liquid carrier, e.g., water, to be easily measured for administration.

Alternatively, liquid or semi-solid oral formulations may be prepared by dissolving or dispersing the active compound (i.e., copper chelator, copper chelator inducing agent, and/or anticancer immunotherapy agent) or salt in vegetable oils, glycols, triglycerides, propylene glycol esters (e.g., propylene carbonate) and other such carriers, and encapsulating these solutions or suspensions in hard or soft gelatin capsule shells. Other useful formulations include those set forth in U.S. Pat. Nos. RE28,819 and 4,358,603. Briefly, such formulations include, but are not limited to, those containing a compound provided herein, a dialkylated mono- or poly-alkylene glycol, including, but not limited to, 1,2-dimethoxymethane, diglyme, triglyme, tetraglyme, polyethylene glycol-350-dimethyl ether, polyethylene glycol-550-dimethyl ether, polyethylene glycol-750-dimethyl ether wherein 350, 550 and 750 refer to the approximate average molecular weight of the polyethylene glycol, and one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, thiodipropionic acid and its esters, and dithiocarbamates.

Other formulations include, but are not limited to, aqueous alcoholic solutions including a pharmaceutically acceptable acetal. Alcohols used in these formulations are any pharmaceutically acceptable water-miscible solvents having one or more hydroxyl groups, including, but not limited to, propylene glycol and ethanol. Acetals include, but are not limited to, di(lower alkyl) acetals of lower alkyl aldehydes such as acetaldehyde diethyl acetal.

### Injectables, Solutions and Emulsions

Parenteral administration, in one embodiment characterized by injection, either subcutaneously, intramuscularly, intraperitoneally or intravenously is also contemplated herein. Injectables can be prepared in conventional forms, either as liquid solutions or suspensions, solid forms suitable for solution or suspension in liquid prior to injection, or as emulsions. The injectables, solutions and emulsions also contain one or more excipients. Suitable excipients are, for example, water, saline, dextrose, glycerol or ethanol. In addition, if desired, the pharmaceutical compositions to be administered may also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins.

Implantation of a slow-release or sustained-release system, such that a constant level of dosage is maintained is also contemplated herein. Briefly, a compound (i.e. copper chelator, copper chelator inducing agent, and/or anticancer immunotherapy agent) provided herein is dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The compound diffuses through the outer polymeric membrane in a release rate controlling step. The percentage of active compound contained in such parenteral compositions is highly dependent on the specific nature thereof, as well as the activity of the compound and the needs of the subject.

Parenteral administration of the compositions includes intravenous, subcutaneous and intramuscular administrations. Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, olive oil, cottonseed oil, corn oil, sesame oil and peanut oil. Antimicrobial agents in bacteriostatic or fungistatic concentrations must be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcellulose, hydroxypropyl methylcellulose and polyvinylpyrrolidone. Emulsifying agents include Polysorbate 80 (TWEEN® 80). A sequestering or chelating agent of metal ions include ethylenediaminetetraacetic acid (EDTA). Pharmaceutical carriers also include ethyl alcohol, polyethylene glycol and propylene glycol for water miscible vehicles; and sodium hydroxide, hydrochloric acid, citric acid or lactic acid for pH adjustment.

The concentration of the pharmaceutically active compound (i.e., copper chelator, copper chelator inducing agent, and/or anticancer immunotherapy agent) is adjusted so that an injection provides an effective amount to produce the desired pharmacological effect. The exact dose depends on the age, weight and condition of the patient or animal as is known in the art.

The unit-dose parenteral preparations may be packaged in an ampule, a vial or a syringe with a needle. All preparations for parenteral administration must be sterile, as is known and practiced in the art.

Illustratively, intravenous or intraarterial infusion of a sterile aqueous solution containing an active compound (i.e., copper chelator, copper chelator inducing agent, and/or anticancer immunotherapy agent) is an effective mode of administration. Another embodiment is a sterile aqueous or oily solution or suspension containing an active material injected as necessary to produce the desired pharmacological effect.

Injectables are designed for local and systemic administration. In one embodiment, a therapeutically effective dosage is formulated to contain a concentration of at least about 0.1% w/w up to about 90% w/w or more, in certain embodiments more than 1% w/w of the active compound (i.e., copper chelator, copper chelator inducing agent, and/or anticancer immunotherapy agent) to the treated tissue(s).

The copper chelator, copper chelator inducing agent, and/or anticancer immunotherapy agent may be suspended in micronized or other suitable form or may be derivatized to produce a more soluble active product or to produce a prodrug. The form of the resulting mixture depends upon a number of factors, including the intended mode of administration and the solubility of the copper chelator, copper chelator inducing agent, and/or anticancer immunotherapy agent in the selected carrier or vehicle. The effective concentration is sufficient for ameliorating the symptoms of the condition and may be empirically determined.

### Lyophilized Powders

Of interest herein are also lyophilized powders, which can be reconstituted for administration as solutions, emulsions and other mixtures. They may also be reconstituted and formulated as solids or gels.

The sterile, lyophilized powder may be prepared by dissolving a copper chelator, copper chelator inducing agent, and/or anticancer immunotherapy agent provided herein, or a pharmaceutically acceptable derivative thereof, in a suitable solvent. The solvent may contain an excipient which improves the stability or other pharmacological component of the powder or reconstituted solution, prepared from the powder. Excipients that may be used include, but are not limited to, dextrose, sorbital, fructose, corn syrup, xylitol, glycerin, glucose, sucrose or other suitable agent. The solvent may also contain a buffer, such as citrate, sodium or potassium phosphate or other such buffer known to those of skill in the art at, in one embodiment, about neutral pH. Subsequent sterile filtration of the solution followed by lyophilization under standard conditions known to those of skill in the art provides the desired formulation. In one embodiment, the resulting solution will be apportioned into vials for lyophilization. Each vial will contain a single dosage or multiple dosages of the compound. The lyophilized powder can be stored under appropriate conditions, such as at about 4 °C to room temperature.

Reconstitution of this lyophilized powder with water for injection provides a formulation for use in parenteral administration. For reconstitution, the lyophilized powder is added to sterile water or other suitable carrier. The precise amount depends upon the selected compound. Such amount can be empirically determined.

### Topical Administration

Topical mixtures are prepared as described for the local and systemic administration. The resulting mixture may be a solution, suspension, emulsions or the like and are formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

The copper chelator, copper chelator inducing agent, and/or anticancer immunotherapy agent or pharmaceutically acceptable derivatives thereof may be formulated as aerosols for topical application, such as by inhalation. These formulations for administration to the respiratory tract can be in the form of an aerosol or solution for a nebulizer, or as a microfine powder for insufflation, alone or in combination with an inert carrier such as lactose. In such a case, the particles of the formulation will, in one embodiment, have diameters of less than 50 microns, in one embodiment less than 10 microns.

The copper chelator, copper chelator inducing agent, and/or anticancer immunotherapy agent may be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the active compound (i.e. copper chelator, copper chelator inducing agent, and/or anticancer immunotherapy agent) alone or in combination with other pharmaceutically acceptable excipients can also be administered.

These solutions, particularly those intended for ophthalmic use, may be formulated as 0.01%-10% (vol. %) isotonic solutions, pH about 5-7, with appropriate salts.

### Compositions for Other Routes of Administration

Other routes of administration, such as transdermal patches, including iontophoretic and electrophoretic devices, vaginal and rectal administration, are also contemplated herein.

Transdermal patches, including iontophoretic and electrophoretic devices, are well known to those of skill in the art. For example, pharmaceutical dosage forms for rectal administration are rectal suppositories, capsules and tablets for systemic effect. Rectal suppositories used herein mean solid bodies for insertion into the rectum which melt or soften at body temperature releasing one or more pharmacologically or therapeutically active ingredients. Pharmaceutically acceptable substances utilized in rectal suppositories are bases or vehicles and agents to raise the melting point. Examples of bases include cocoa butter (theobroma oil), glycerin-gelatin, carbowax (polyoxyethylene glycol) and appropriate mixtures of mono-, di- and triglycerides of fatty acids. Combinations of the various bases may be used. Agents to raise the melting point of suppositories include spermaceti and wax. Rectal suppositories may be prepared either by the compressed method or by molding. The weight of a rectal suppository, in one embodiment, is about 2 to 3 gm.

Tablets and capsules for rectal administration are manufactured using the same pharmaceutically acceptable substance and by the same methods as for formulations for oral administration.

### Targeted Formulations

The copper chelator, copper chelator inducing agent, and/or anticancer immunotherapy agent provided herein, or pharmaceutically acceptable derivatives thereof, may also be formulated to be targeted to a particular tissue, receptor, or other area of the body of the subject to be treated. Many such targeting methods are well known to those of skill in the art. All such targeting methods are contemplated herein for use in the instant compositions.

In one embodiment, liposomal suspensions, including tissue-targeted liposomes, such as tumor-targeted liposomes, may also be suitable as pharmaceutically acceptable carriers. These may be prepared according to methods known to those skilled in the art. For example, liposome formulations may be prepared as described in U.S. Pat. No. 4,522,811. Briefly, liposomes such as multilamellar vesicles (MLVs) may be formed by drying down egg phosphatidyl choline and brain phosphatidyl serine (7:3 molar ratio) on the inside of a flask. A solution of a compound provided herein in phosphate buffered saline lacking divalent cations (PBS) is added and the flask shaken until the lipid film is dispersed. The resulting vesicles are washed to remove unencapsulated compound, pelleted by centrifugation, and then resuspended in PBS.

### Co-Administration With Other Drugs

In accordance with another aspect of the present invention, it is contemplated that copper chelators, copper chelator inducing agents, and/or anticancer immunotherapy agents as described herein may be administered to a subject in need thereof in combination with medication considered by those of skill in the art to be current standard of care for the condition of interest. Such combinations provide one or more advantages to the subject, e.g., requiring reduced dosages to achieve similar benefit, obtaining the desired palliative effect in less time, and the like.

Copper chelators, copper chelator inducing agents, and/or anticancer immunotherapy agents in accordance with the present invention may be administered as part of a therapeutic regimen with other drugs. It may be desirable to administer a combination of active compounds, for example, for the purpose of treating a particular disease or condition. Accordingly, it is within the scope of the present invention that two or more pharmaceutical compositions, at least one of which contains a copper chelator, copper chelator inducing agent, and/or anticancer immunotherapy agent according to the present invention, may be combined in the form of a kit suitable for co-administration of the compositions.

In one embodiment of the copper chelator, copper chelator inducing agent, and/or anticancer immunotherapy agent may be administered with a second therapeutic agent. In one embodiment the second therapeutic agent is an anti-cancer agent.

When two or more active ingredients are co-administered, the active ingredients may be administered simultaneously, sequentially or separately. In one embodiment the copper chelator, copper chelator inducing agent, and/or anticancer immunotherapy agent are co-administered simultaneously with another therapeutic agent. In another embodiment the copper chelator, copper chelator inducing agent, and/or anticancer immunotherapy agent and the other therapeutic agent are administered sequentially. In a further embodiment the copper chelator, copper chelator inducing agent, and/or anticancer immunotherapy agent and the other therapeutic agent are administered separately.

In an eighth aspect of the invention there is provided a pharmaceutical composition comprising a copper chelator or a copper chelator inducing agent, and an anticancer immunotherapy agent.

The following options may be used in conjunction with the eighth aspect, either individually or in any combination.

The pharmaceutical composition may comprise a pharmaceutically acceptable carrier or excipient.

The copper chelator, copper chelator inducing agent, anticancer immunotherapy agent, and/or pharmaceutical composition may be as hereinbefore described with respect to the first, second, third, fourth, fifth, sixth, or seventh aspect.

The pharmaceutical composition of the eighth aspect may be used in the method of the first aspect. The method of the first aspect may use the pharmaceutical composition of the eighth aspect.

The pharmaceutical composition of the eighth aspect may be used in the use of the second aspect. The use of the second aspect may use the pharmaceutical composition of the eighth aspect.

The pharmaceutical composition of the eighth aspect may be used in the use of the third aspect. The use of the third aspect may use the pharmaceutical composition of the eighth aspect.

The pharmaceutical composition of the eighth aspect may be used in the use of the fourth aspect. The use of the fourth aspect may use the pharmaceutical composition of the eighth aspect.

The pharmaceutical composition of the eighth aspect may include the copper chelator or copper chelator inducing agent of the fifth aspect. The copper chelator or copper chelator inducing agent of the fifth aspect may be a component of the pharmaceutical composition of the eighth aspect.

The pharmaceutical composition of the eighth aspect may include the anticancer immunotherapy agent of the sixth aspect. The anticancer immunotherapy agent of the sixth aspect may be a component of the pharmaceutical composition of the eighth aspect.

The pharmaceutical composition of the eighth aspect may include the kit or combination of the seventh aspect. The kit or combination of the seventh aspect may be a component of the pharmaceutical composition of the eighth aspect.

Disclosed herein are the following forms:
1. A method for boosting the anti-cancer immune response and efficacy of immunotherapies in a subject having a tumor, comprising administering to the subject a copper chelator in an amount effective to chelate copper from the tumor microenvironment, thereby reducing copper-mediated immunosuppression and enhancing the anti-cancer immune response and efficacy of immunotherapies against the tumor.
2. The method of form 1, wherein the copper chelator is selected from the group consisting of tetrathiomolybdate, trientine, penicillamine, D-penicillamine and Zinc salts.
3. The method of form 1, wherein the subject has a solid tumor or a hematological malignancy.
4. The method of form 1, wherein the immunotherapy is selected from the group consisting of immune checkpoint inhibitors (e.g. α-CTLA4 targeting ICI and/or α-PDL1 targeting ICI), chimeric antigen receptor (CAR) T-cell therapy, bispecific T-cell engagers (BiTEs), (CAR) NK-cell therapy, adoptive cell therapy and antibody-dependent cell cytotoxicity (ADCC).
5. The method of form 1, wherein the copper chelator is administered before, concurrently with, or after the immunotherapy.
6. A pharmaceutical composition comprising a copper chelator and a pharmaceutically acceptable carrier or excipient, wherein the copper chelator is present in an amount effective to chelate copper from the tumor microenvironment, thereby reducing copper-mediated immunosuppression and enhancing the anti-cancer immune response and efficacy of immunotherapies against a tumor.
7. The pharmaceutical composition of form 6, wherein the copper chelator is selected from the group consisting of tetrathiomolybdate, trientine, penicillamine, D-penicillamine and Zinc salts.
8. The pharmaceutical composition of form 6, further comprising an immunotherapy selected from the group consisting of immune checkpoint inhibitors (e.g. α-CTLA4 targeting ICI and/or α-PDL1 targeting ICI), chimeric antigen receptor (CAR) T-cell therapy, bispecific T-cell engagers (BiTEs), (CAR) NK-cell therapy, adoptive cell therapy and antibody-dependent cell cytotoxicity (ADCC).
9. A method of treating a subject having a tumor, comprising administering the pharmaceutical composition of form 6 to the subject in an amount effective to chelate copper from the tumor microenvironment, thereby reducing copper-mediated immunosuppression and enhancing the anti-cancer immune response and efficacy of immunotherapies against the tumor.
10. The method of form 9, wherein the subject has a solid tumor or a hematological malignancy.
11. The method of form 9, wherein the immunotherapy is selected from the group consisting of immune checkpoint inhibitors (e.g. α-CTLA4 targeting ICI and/or α-PDL1 targeting ICI), chimeric antigen receptor (CAR) T-cell therapy, bispecific T-cell engagers (BiTEs), (CAR) NK-cell therapy, adoptive cell therapy and antibody-dependent cell cytotoxicity (ADCC).
12. The method of form 9, wherein the copper chelator is administered before, concurrently with, or after the immunotherapy.
13. A method for boosting the anti-cancer immune response and efficacy of immunotherapies in a subject having a tumor, comprising administering to the subject a copper chelator in an amount effective to chelate copper from the tumor microenvironment, thereby reducing copper-mediated immunosuppression, enhancing the anti-cancer immune response, and activating myeloid cell response against the tumor.
14. The method of form 13, wherein the myeloid cell response is mediated by dendritic cells, macrophages, or neutrophils.
15. The method of form 13, wherein the copper chelator is selected from the group consisting of tetrathiomolybdate, trientine, penicillamine, D-penicillamine and Zinc Salts
16. The method of form 13, wherein the subject has a solid tumor or a hematological malignancy.
17. The method of form 13, wherein the immunotherapy is selected from the group consisting of immune checkpoint inhibitors (e.g. α-CTLA4 targeting ICI and/or α-PDL1 targeting ICI), chimeric antigen receptor (CAR) T-cell therapy, bispecific T-cell engagers (BiTEs), (CAR) NK-cell therapy, adoptive cell therapy and antibody-dependent cell cytotoxicity (ADCC).
18. The method of form 13, wherein the copper chelator is administered before, concurrently with, or after the immunotherapy.
19. A pharmaceutical composition comprising a copper chelator and a pharmaceutically acceptable carrier or excipient, wherein the copper chelator is present in an amount effective to chelate copper from the tumor microenvironment, thereby reducing copper-mediated immunosuppression, enhancing the anti-cancer immune response, and activating myeloid cell response against the tumor.
20. The pharmaceutical composition of form 19, wherein the myeloid cell response is mediated by dendritic cells, macrophages, or neutrophils.
21. The pharmaceutical composition of form 19, wherein the copper chelator is selected from the group consisting of tetrathiomolybdate, trientine, penicillamine, D-penicillamine and Zinc Salts.
22. The pharmaceutical composition of form 19, further comprising an immunotherapy selected from the group consisting of immune checkpoint inhibitors (e.g. α-CTLA4 targeting ICI and/or α-PDL1 targeting ICI), chimeric antigen receptor (CAR) T-cell therapy, bispecific T-cell engagers (BiTEs), (CAR) NK-cell therapy, adoptive cell therapy and antibody-dependent cell cytotoxicity (ADCC).
23. A method of treating a subject having a tumor, comprising administering the pharmaceutical composition of form 19 to the subject in an amount effective to chelate copper from the tumor microenvironment, thereby reducing copper-mediated immunosuppression, enhancing the anti-cancer immune response, and activating myeloid cell response against the tumor.
24. The method of form 23, wherein the myeloid cell response is mediated by dendritic cells, macrophages, or neutrophils.
25. The method of form 23, wherein the subject has a solid tumor or a hematological malignancy.
26. The method of form 23, wherein the immunotherapy is selected from the group consisting of immune checkpoint inhibitors (e.g. α-CTLA4 targeting ICI and/or α-PDL1 targeting ICI), chimeric antigen receptor (CAR) T-cell therapy, bispecific T-cell engagers (BiTEs), (CAR) NK-cell therapy, adoptive cell therapy and antibody-dependent cell cytotoxicity (ADCC).
27. The method of form 23, wherein the copper chelator is administered before, concurrently with, or after the immunotherapy
28. A method for increasing the efficacy of Antibody-dependent cell cytotoxicity (ADCC) in a subject having a tumor, comprising administering to the subject a copper chelator in an amount effective to chelate copper from the tumor microenvironment, thereby reducing copper-mediated immunosuppression, enhancing the anti-cancer immune response, and increasing the efficacy of ADCC.
29. The method of form 28, wherein the copper chelator is selected from the group consisting of tetrathiomolybdate, trientine, penicillamine, D-penicillamine and Zinc Salts.
30. The method of form 28, wherein the subject has a solid tumor or a hematological malignancy.
31. The method of form 28, wherein the ADCC is mediated by natural killer (NK) cells, monocytes, or macrophages.
32. The method of form 28, wherein the copper chelator is administered before, concurrently with, or after the administration of the antibody used in the ADCC.
33. A pharmaceutical composition comprising a copper chelator and a pharmaceutically acceptable carrier or excipient, wherein the copper chelator is present in an amount effective to chelate copper from the tumor microenvironment, thereby reducing copper-mediated immunosuppression, enhancing the anti-cancer immune response, and increasing the efficacy of ADCC.
34. The pharmaceutical composition of form 33, wherein the copper chelator is selected from the group consisting of tetrathiomolybdate, trientine, penicillamine, D-penicillamine and Zinc Salts.
35. The pharmaceutical composition of form 33, further comprising an antibody that mediates ADCC.
36. The pharmaceutical composition of form 33, wherein the ADCC is mediated by natural killer (NK) cells, monocytes, or macrophages.
37. A method of treating a subject having a tumor, comprising administering the pharmaceutical composition of form 33 to the subject in an amount effective to chelate copper from the tumor microenvironment, thereby reducing copper-mediated immunosuppression, enhancing the anti-cancer immune response, and increasing the efficacy of ADCC.
38. The method of form 37, wherein the copper chelator is selected from the group consisting of tetrathiomolybdate, trientine, penicillamine, D-penicillamine and Zinc Salts
39. The method of form 37, wherein the subject has a solid tumor or a hematological malignancy.
40. The method of form 37, wherein the ADCC is mediated by natural killer (NK) cells, monocytes, or macrophages.
41. The method of form 37, wherein the copper chelator is administered before, concurrently with, or after the administration of the antibody used in the ADCC.

### BRIEF DESCRIPTION OF THE DRAWINGS

The patent or application file contains at least one drawing executed in colour. Copies of this patent or patent application publication with colour drawings will be provided by the Office upon request and payment of the necessary fee (if appropriate).

FIG. 1: an illustration of a tumor microenvironment.

FIG. 2: formation of a TEPA Cu²⁺ complex.

FIG. 3: a graphical depiction of Example 1 disclosed herein.

FIG. 4: single cell RNA sequencing of tumour cells.

FIG. 5: graph showing MYCN oncogene downregulation.

FIG. 6: single cell RNA sequencing of infiltrating immune cells. This diagram also shows that copper chelation induced a near five-fold increase in neutrophil (the most abundant leukocyte type in circulation) infiltration into the tumour microenvironment.

FIG. 7: Heatmap showing that the major genes responsible for copper uptake are highly expressed only in the neutrophils.

FIG. 8: GSEA analysis demonstrates that copper chelation polarises infiltrating neutrophils towards an “N1” phenotype.

FIG. 9: Opal multiplex analysis.

FIG. 10: Opal multiplex analysis summary showing increased infiltration of CD11B+ myeloid cells in addition to increased NCR1+ natural killer cell and CD8+ cytotoxic T cells into the tumour microenvironment with copper chelation.

FIG. 11: single cell RNAseq network.

FIG. 12: Cytokines assay showing that copper chelation increases levels of pro-inflammatory cytokines in the tumour.

FIG. 13: in vitro model of tumour cells (SK-N-BE(2)C)/CD45+ immune cell co-culture. FIG. 13A shows that the copper chelator TEPA removes copper from the tumour cells (SK-N-BE(2)C) and it is released in the media where is fully absorbed by the CD45+ immune cell. FIG. 13B shows that neutrophils are able to uptake the copper released by the cancer cells in the media in response to copper chelation therapy.

FIG. 14: tissue sections stained with a copper-specific probe to determine intracellular copper distribution on a single cell basis. This experiment showed copper decreases on the tumour and increases in the immune cells after copper chelation treatment.

FIG. 15: dosage schedule for TEPA or saline, and vehicle, IgG, or anti-GD2 moAb Example 3. TEPA was given first at 400 mg/kg/day for 5 days as a single agent (pre-treatment); thereafter five 7-day treatment cycles were performed where TEPA 400 mg/kg/day was administered for five days and anti-GD2 was administered at day one and day five in combination. After the fifth 7-day cycle, the treatment was stopped.

FIG. 16: Opal multiplex immunohistochemistry for Example 3.

FIG. 17: Opal multiplex immunohistochemistry showing increased CD8/NCR1/CD11B infiltration when TEPA was combined with anti-GD2 therapy.

FIG. 18: mouse survival (A) and weight (B) for Example 3.

FIG. 19: structural comparison of TEPA and TETA and their complexes with Cu²⁺.

FIG. 20: dosage schedule for TETA or saline, and vehicle, IgG, or anti-GD2 moAb Example 4.

FIG. 21: Opal multiplex immunohistochemistry for Example 4.

FIG. 22: Opal multiplex immunohistochemistry showing increased CD8/NCR1/CD11B infiltration when TETA was combined with anti-GD2 therapy.

FIG. 23: mouse survival (A) and weight (B) for Example 4.

FIG. 24: Structure of TETA Cu²⁺ complex.

FIG. 25: Equilibrium status of TETA by pH.

FIG. 26: Structure of TEPA Cu²⁺ complex.

FIG. 27: Copper chelation potentiates anti-tumor activity of anti-GD2 immunotherapy. a) Experimental design and dosing strategy (IP, intraperitoneal; p.o., oral gavage). b) Kaplan-Meier survival curves of Th-MYCN mice (n=10/group). c) Statistical comparisons using the Mantel-Cox log-rank test (two-tailed) of survival curves presented in b. d) Representative images of merged OPAL multiplex immunofluorescence spectra depicting the tumoral distribution of NCR1+ natural killer cells (red), CD8+ cytotoxic T cells (yellow) and CD11b+ myeloid (white) in Th-MYCN neuroblastoma tumor tissue 14 days post-treatment. Scale bar, 100 μm. e) Immune cell quantification of panel d as positive counts per 1000 nuclei. Data presented as mean±SEM (n=3-4/group). Significance was calculated using a one-tailed Mann-Whitney U test (*p≤0.005, **p≤0.01, ***p≤0.001, ns=not significant). Abbreviations: moAb, monoclonal antibody.

FIG. 28: Copper chelation promotes an immune-permissive tumor microenvironment. a) Experimental design and dosing strategy. b) Cytokine levels in tumor lysates obtained from control and TEPA-treated Th-MYCN mice. Data represented as mean±SEM (n=4-11/group). Significance was calculated using the Mann-Whitney U test (*p≤0.05; **p≤0.01). c) Representative images of merged OPAL multiplex immunofluorescence spectra depicting the tumoral distribution of NCR1+ natural killer cells (red), CD8+ cytotoxic T cells (yellow) and CD11b+ myeloid (white) in Th-MYCN neuroblastoma tumor tissue after one week of treatment. Scale bar, 100 μm. d) Immune cell quantification of panel c as counts per 1000 nuclei. Data represented as mean±SEM (n=3-5/group). Significance was calculated using a Mann-Whitney U test (*p≤0.05).

FIG. 29: Copper chelation reinvigorates anti-tumor immunity via pro-inflammatory signaling. a) Representative images of control and TEPA-treated Th-MYCN tumor cores resected after seven days, stained with fluorescently conjugated antibodies to PanCK (red), smooth muscle actin (yellow), CD45 (green) with DAPI nuclear dye. Scale bar, 300 μm. b) Sankey plot of Th-MYCN tumor cores depicting the proportion of CD45-infiltrated false/true regions of interest, plotted against treatment group and duration. c) Volcano plot (log 2 fold-change (FC) vs −log 10 p value) of genes upregulated and downregulated in TEPA-treated infiltrated versus non-infiltrated regions of interest. p, false discovery rate (FDR), adjusted with the Benjamini-Hochberg correction. Thresholds: p<0.1; |Log 2FC|>0.5. d) Overrepresentation analysis of pathways relatively enriched in TEPA-treated treated infiltrated tumor regions compared to non-infiltrated regions, presented as nodes with associated genes as branches. e) Bar plot visualization of the data presented in d.

FIG. 30: The neuroblastoma tumor microenvironment is sensitive to copper chelation therapy and promotes neutrophil infiltration. a) Schematic of experimental design and tumor processing workflow for single-cell RNA sequencing. b) Uniform manifold approximation and projection (UMAP) representation of tumoral compartment (13,544 cells), coloured by treatment group. c) Violin plots of gene expression levels associated with intracellular copper levels (Mt1, Mt2) and neuroblastoma oncogene Mycn, split by treatment group; ***p<0.001. The horizontal line indicates the median of the data. d) Enrichment plot for the HALLMARK_MYC_TARGETS_V1 gene set by gene set enrichment analysis. e) Split UMAP representation of immune cell compartment (12,127 cells total) according to treatment arm and coloured by annotated immune subsets (n=13). f) Bar plot of the proportion of immune cell subsets shown in e. g, Dot plot of gene expression markers associated used to classify the immune subsets defined in e.

FIG. 31: Neutrophils supersede tumorigenic signaling to drive reinvigoration of anti-tumor immunity. a) Overrepresentation analysis of pathways relatively enriched in TEPA-treated immune cell clusters compared to control, presented as nodes with associated genes as branches using single-cell RNA sequencing. b) Circle network diagram of significant cell-cell interaction pathways in control and TEPA-treated tumour datasets. Width of edge represents the interaction strength and edge thickness is proportional to signal strength. c) Scatter plots comparing the outgoing and incoming interaction strength between control and TEPA-treated samples.

FIG. 32: Copper chelation facilitates egress and N1-polarisation of neutrophils via copper mobilization. a) Heatmap comparing the average expression of genes associated with copper metabolism across treatment arms within immune cell clusters. b) Heatmap comparing average expression of genes curated to form N1 (anti-tumor) and N2 (pro-tumor) neutrophil phenotypic signatures. Data presented in panels a and b were obtained from single-cell RNA sequencing with relevant cell values averaged and row-scaled (z-score). c) Gene set enrichment analysis shows top pathways relatively enriched in TEPA-treated neutrophils. “N1 ANTI-TUMOR NEUTROPHILS” signature was constructed using the N1-associated genes listed in b. d) Count and percentage of circulating neutrophils obtained from control and TEPA-treated Th-MYCN mice after seven days. Data represented as mean±SEM (n=4/group). Significance was calculated using the Mann-Whitney U test (*p≤0.05). e) IncuCyte cell imaging of neuroblastoma cell line SK-N-BE(2)-C transfected with a plasmid encoding turbo green fluorescent protein (tGFP)-tagged MT1X protein following 24 hr of TEPA treatment (10× objective). Scale bar, 100 μm. f) Concentration of copper in conditioned media before and after 30 min incubation with naïve neutrophils isolated from healthy donors. Data represented as mean±SEM (n=3). Significance was calculated using a paired t-test (*p≤0.05).

FIG. 33: Copper chelating agent TETA synergizes with anti-GD2 therapy to mediate anti-tumor activity in the syngeneic NXS2 model of neuroblastoma. a) Tumor growth kinetics in a syngeneic model of neuroblastoma involving the subcutaneous inoculation of A/J mice with NXS2 cells. Animals commenced treatment one week after inoculation (black arrow) and were treated by oral gavage with saline (control) or TETA (400 mg/kg/day for seven days before blood and tumor collection (n=4/group). Significance was calculated using the Mann-Whitney U test (*p≤0.05) at each timepoint. Flow cytometric analyses of freshly dissociated tumors gated on b) neutrophil frequency and c) proportion of cells in panel n expressing MMP9 indicating an N1 pro-inflammatory phenotype. Significance was calculated using the Mann-Whitney U test (*p≤0.05). d) Experimental design of the syngeneic NXS2→A/J preclinical model and immunocombination dosing strategy. e-g Arrows indicate treatment period. e) Relative weight change in tumor-bearing mice measured from date of inoculation. f) Relative tumor volume measured from date of inoculation. g) Survival curves of tumor-bearing mice measured from date of inoculation. h) Statistical pairwise comparisons of survival curves in panel g calculated using the Mantel-Cox log-rank test (two-tailed). Data are representative of one experiment (n=8-10/group). i) Representative images of merged OPAL multiplex immunofluorescence spectra depicting the tumoral distribution of NCR1+ natural killer cells (red), CD8+ cytotoxic T cells (yellow) and CD11b+ myeloid (white) in NXS2 neuroblastoma tumor tissue after 14 days post-treatment. Scale bar, 100um. j) Immune cell quantification of panel i as counts per 1000 nuclei (n=3-4/group). Significance was calculated using a one-tailed Mann-Whitney U test (*p≤0.005, **p≤0.01, ***p≤0.001, ns: not significant). Abbreviations: IP, intraperitoneal; moAb, monoclonal antibody; p.o., oral gavage.

FIG. 34: Gene set enrichment analyses for immune clusters determined by single-cell RNA sequencing for a) B cells, b) basophils, c) CD4+ naïve T cells, d) CD4+ memory T cells, e) CD8+ naïve T cells, f) CD8+ NKT-like cells, g) dendritic cells, h) double-negative regulatory T cells, i) macrophages, j) eosinophils and, k) natural killer cells. The gamma-delta T cell cluster was excluded from analysis due to low cell numbers.

FIG. 35: Top pathways determined by gene set enrichment analysis for immune clusters determined by single-cell RNA sequencing. Bar plot displaying enriched pathways in infiltrating immune cells in TEPA-treated compared to control tumors. Bars are associated with individual immune clusters as specified in the legend.

FIG. 36: Heatmaps of top signaling networks contributing to outgoing or incoming signalling of the different immune cell clusters in a) control and b) TEPA-treated tumors. The signal relative strength is represented as shades of green, the upper horizontal bar indicates the number of outgoing (left) and incoming (right) interactions per immune cell cluster, the right vertical bar indicates the relative strength of a certain signalling network.

FIG. 37: Dot plots illustrating the most significant a) outgoing and, b) incoming interactions between neutrophils and the other immune cell clusters expressed as ligand-receptor pairs between control (grey) and TEPA-treated (red) groups.

FIG. 38: Tumoral immune infiltration is associated with higher transcriptional levels of copper exporter ATP7A. Box and whisker plots of transcripts per million (TPM) of ATP7A and respective association with Immune Paediatric Signature Score (IPASS) status of solid pediatric cancers.

FIG. 39: Copper chelating agent TETA is a safe and non-toxic therapeutic strategy. Hematological analyses of metabolites associated with a) hepatic, b) renal, and c) systemic toxicities after one week of treatment. Data represented as mean±SEM (n=3/group). Significance was calculated using the Mann-Whitney U test (ns, not significant).

FIG. 40: a) Flow cytometric gating strategy used for single-cell RNA sequencing in Th-MYCN tumors to illustrate the selection of viable NK1.1+ natural killer cells, CD3+ T cells, CD11b+ myeloid cells and the tumor compartment. b) Gating strategy used to determine viable CD45+CD11b+Ly6G+ neutrophil frequencies in preclinical NXS2 tumors.

FIG. 41: EZH2 inhibition is emerging as an exciting avenue to increase GD2 antigen expression in neuroblastoma and Ewing's sarcoma. The copper chelator TEPA robustly downregulates EZH2 in diffuse midline glioma (DMG), an aggressive childhood brain cancer (a) and neuroblastoma cell lines (b). Translating this work in vivo, it was found that TEPA robustly upregulated GD2 expression in Th-MYCN neuroblastoma tumours after one week of treatment using flow cytometry (c). Interestingly, preliminary data from a xenograft model of glioblastoma demonstrated that one week of TEPA pre-treatment had a synergistic effect in reducing tumour growth in combination with GD2-targeting CAR T cells (1×10⁶ cells IV in one injection) (d). This data strongly suggests copper chelation can epigenetically modulate GD2 antigen expression and thereby increase sensitivity to GD2 CAR T immunotherapy. Importantly, those results support the idea that by reducing intra-tumoural copper we can simultaneously reduce immune evasion mechanisms in the TME whilst increasing the target of the GD2-CAR T in the cancer cells.

FIG. 42: Dosage regime for copper chelation therapy in mesothelioma models. Animals received 2 weeks of daily doses of 800 mg per kg TETA orally, followed by alternative day dosing until end of experiment.

FIG. 43: Tumor growth data for the AB1-HA mesothelioma model combination treatment with immune checkpoint inhibitor (ICI) and copper chelator (TETA): a) PBS alone; b) α-CTLA4 targeting ICI and α-PDL1 targeting ICI; c) TETA alone; d) combination of TETA, α-CTLA4 targeting ICI and α-PDL1 targeting ICI.

FIG. 44: Animal survival data for the AB1-HA mesothelioma model combination treatment with immune checkpoint inhibitor (ICI) and copper chelator (TETA).

FIG. 45: Tumor growth data for the AE17-OVA mesothelioma model combination treatment with immune checkpoint inhibitor (ICI) and copper chelator (TETA): a) PBS alone; b) α-CTLA4 targeting ICI and α-PDL1 targeting ICI; c) TETA alone; d) combination of TETA, α-CTLA4 targeting ICI and α-PDL1 targeting ICI.

FIG. 46: Animal survival data for the AE17-OVA mesothelioma model combination treatment with immune checkpoint inhibitor (ICI) and copper chelator (TETA).

FIG. 47: Flow cytometry results with PBS or TETA showing proportion of cells as a percentage of viable CD45+ve: CD3−ve (top) and CD3+ve (bottom).

FIG. 48: Flow cytometry results with PBS or TETA showing proportion of cells as a percentage of viable CD45+ve: CD4+ve (top) and CD8+ve (bottom).

FIG. 49: Flow cytometry results with PBS or TETA showing proportion of cells as a percentage of viable CD45+ve: CD4+ve (top) and FOXP3 CD25+ve (bottom).

FIG. 50: Flow cytometry results with PBS or TETA showing proportion of cells as a percentage of viable CD45+ve: NKP46+ve (top) and Ly6G CD11b+ve (bottom).

FIG. 51: Flow cytometry results with PBS or TETA showing proportion of cells as a percentage of viable CD45+ve: NKP46+ve (top) and NKP46 CD11b+ve (bottom).

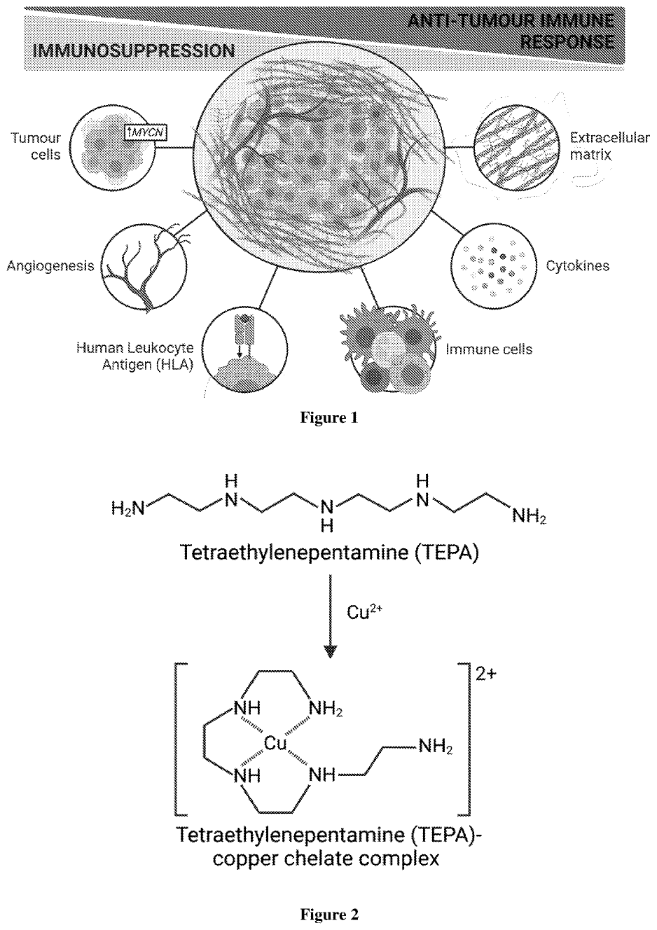

### DEFINITIONS

In describing and claiming the present invention, the following terminology will be used in accordance with the definitions set out below. It is also to be understood that the terminology used herein is for the purpose of describing particular embodiments of the invention only and is not intended to be limiting.

Unless defined otherwise, all technical and scientific terms used herein have the same meaning as commonly understood by one having ordinary skill in the art to which the invention pertains.

Unless the context clearly requires otherwise, throughout the description and the claims, the terms “comprise”, “comprising”, and the like are to be construed in an inclusive sense as opposed to an exclusive or exhaustive sense; that is to say, in the sense of “including, but not limited to”. For example, a composition, mixture, process or method that comprises a list of elements is not necessarily limited to only those elements but may include other elements not expressly listed or inherent to such composition, mixture, process or method.

The transitional phrase “consisting of” excludes any element, step, or ingredient not specified. If in the claim, such would close the claim to the inclusion of materials other than those recited except for impurities ordinarily associated therewith. When the phrase “consisting of” appears in a clause of the body of a claim, rather than immediately following the preamble, it limits only the element set forth in that clause; other elements are not excluded from the claim as a whole.

The transitional phrase “consisting essentially of” is used to define a composition, process or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Where applicants have defined an invention or a portion thereof with an open-ended term such as “comprising”, it should be readily understood that (unless otherwise stated) the description should be interpreted to also describe such an invention using the terms “consisting essentially of” or “consisting of”. In other words, with respect to the terms “comprising”, “consisting of”, and “consisting essentially of”, where one of these three terms is used herein, the presently disclosed and claimed subject matter may include the use of either of the other two terms. Thus, in some embodiments not otherwise explicitly recited, any instance of “comprising” may be replaced by “consisting of” or, alternatively, by “consisting essentially of”.

Other than in the claims or operating examples, or where otherwise indicated, all numbers expressing quantities of ingredients or reaction conditions used herein are to be understood as modified in all instances by the term “about”. The examples are not intended to limit the scope of the invention.

In what follows, or where otherwise indicated, “%” will mean “weight %”, “ratio” will mean “weight ratio” and “parts” will mean “weight parts”.

The terms “predominantly”, “predominant”, and “substantially” as used herein shall mean comprising more than 50% by weight, unless otherwise indicated.

As used herein, with reference to numbers in a range of numerals, the terms “about,” “approximately” and “substantially” are understood to refer to the range of −10% to +10% of the referenced number, preferably −5% to +5% of the referenced number, more preferably −1 % to +1% of the referenced number, most preferably −0.1% to +0.1% of the referenced number. Moreover, with reference to numerical ranges, these terms should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, from 8 to 10, and so forth.

The terms “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

As used herein, the term “copper chelator” means an organic molecule having more than atom which is capable of forming a coordinate bond with a copper ion, optionally with a Cu¹⁺ and/or Cu²⁺ ion. In certain embodiments the copper chelator may form a copper complex with the copper ion that has a stability constant of greater than 2, optionally greater than 5. Example copper chelators that are known in the art include: ethylenediamine, polyethylenimine and derivatives thereof, tetrathiomolybdate; N,N′-bis(2-aminoethyl)ethane-1,2-diamine (trientine, triethylenetetramine, TETA); penicillamine; D-penicillamine; N′-(2-aminoethyl)-N-[2-(2-amino ethylamino) ethyl]ethane-1,2-diamine (tetraethylenepentamine, TEPA); 2-[2-[bis(carboxymethyl)amino]ethyl-(carboxymethyl)amino]acetic acid (EDTA); 2-[bis[2-[bis(carboxymethyl)amino]ethyl] amino]acetic acid (DTPA); N,N,N′,N′-tetrakis(2-pyridyl-methyl) ethylenediamine (TPEN); 2-[4,8,11-tris(carboxymethyl)-1,4,8,11-tetrazacyclotetradec-1-yl]acetic acid; 2-[4,7,10-tris(carboxymethyl)-1,4,7,10-tetrazacyclododec-1-yl]acetic acid (DOTA); (2S)-2-amino-3-methyl-3-sulfanylbutanoic acid (D-pen); bis(sulfanylidene)molybdenum sulfanide (TTM); 5-chloro-7-iodo-8-hydroxy-quinoline (Cliquinol); diethylcarbamodithioic acid (DDC); 2,9-dimethyl-1,10-phenanthroline (Neocuproine); 2,9-dimethyl-4,7-diphenyl-1,10-phenanthroline (Bathocuproine); 4-[2,9-dimethyl-7-(4-sulfophenyl)-1,10-phenanthrolin-4-yl]benzenesulfonic acid (BCS); and N,N′-bis(cyclohexylideneamino)oxamide (Cuprizone).

As used herein, the term “copper chelator inducing agent” means a substance, which when administered to a subject, causes the subject to produce a copper chelator, or increase its production of a copper chelator. Example copper chelator inducing agents which are known in the art include zinc salts, which when administered to a mammal induce the production of metallothionein (which itself is a copper chelator).

As used herein, the term “anticancer immunotherapy agent” means an agent which is used to treat cancer by stimulating or suppressing the immune system of a subject. Example anticancer immunotherapy agents which are known in the art include: antibodies, immune checkpoint inhibitors, chimeric antigen receptor (CAR) T-cells, bispecific T-cell engagers (BiTEs), (CAR) NK-cells, adoptive immune cells and antibody-dependent immune cells.

### ABBREVIATIONS

Antibody-dependent cell cytotoxicity (ADCC); 4-[2,9-dimethyl-7-(4-sulfophenyl)-1,10-phenanthrolin-4-yl]benzenesulfonic acid (BCS); butylated hydroxyanisole (BHA); butylated hydroxytoluene (BHT); bispecific T-cell engagers (BiTEs); chimeric antigen receptor (CAR); diethylcarbamodithioic acid (DDC); diffuse intrinsic pontine glioma (DIPG); diffuse midline glioma (DMG); dimethyl sulfoxide (DMSO); 2-[4,7,10-tris(carboxymethyl)-1,4,7,10-tetrazacyclododec-1-yl]acetic acid (DOTA); (2S)-2-amino-3-methyl-3-sulfanylbutanoic acid (D-pen); 2-[bis[2-[bis(carboxymethyl)amino]ethyl] amino]acetic acid (DTPA); ethylenediaminetetraacetic acid (EDTA); US Food and Drug Administration (FDA); gene set enrichment analysis (GSEA); immune checkpoint inhibitor(s) (ICI); monoclonal antibodies (mAbs); multilamellar vesicles (MLVs); natural killer cells (NK cells); phosphate buffered saline (PBS); programmed death receptor 1 (PD-1); programmed death ligand 1 (PD-L1); tetraethylenepentamine (TEPA); triethylenetetramine (TETA); N,N,N′,N′-tetrakis(2-pyridyl-methyl) ethylenediamine (TPEN); and bis(sulfanylidene)molybdenum sulfanide (TTM).

Preferred features, embodiments and variations of the invention may be discerned from the following Examples which provide sufficient information for those skilled in the art to perform the invention. The following Examples are not to be regarded as limiting the scope of the preceding Summary of the Invention in any way.

### EXAMPLES

### Example 1: TEPA in TH-MYCN Transgenic Mouse Model

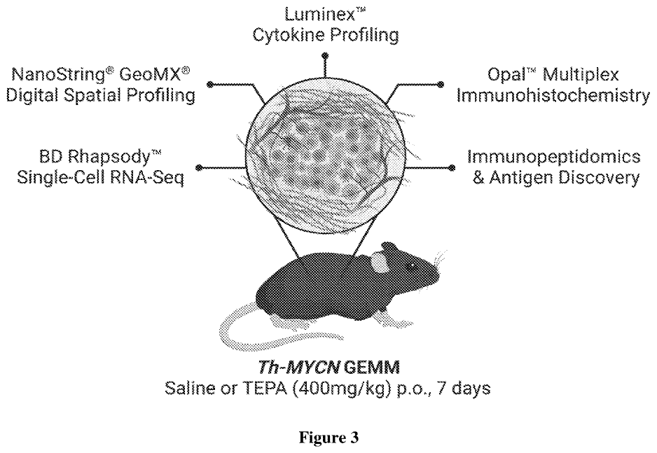

TEPA is a copper chelator, which can form a copper chelate complex with Cu²⁺ (FIG. 2). TEPA was dosed orally to TH-MYCN GEMM transgenic mice (which spontaneously develop aggressive tumors, closely resembling human neuroblastoma in location, histology, biology and cytogenetic abnormalities) at a daily dose of 400 mg/kg for 7 days. The control group received an equivalent volume daily oral saline dose over 7 days (FIG. 3).

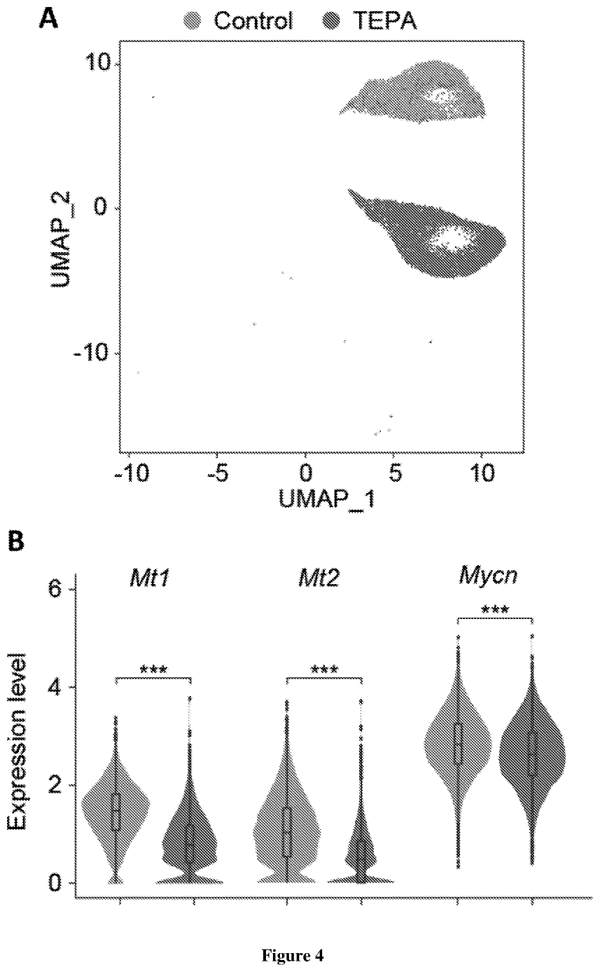

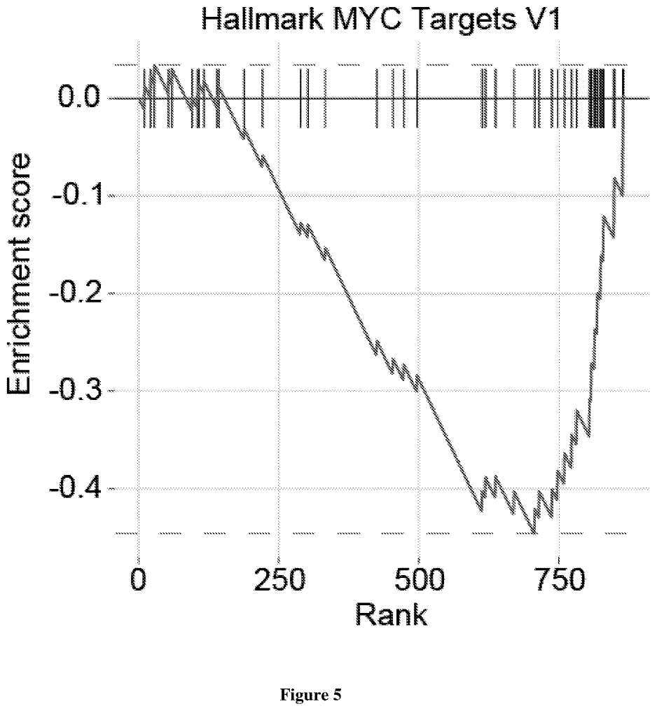

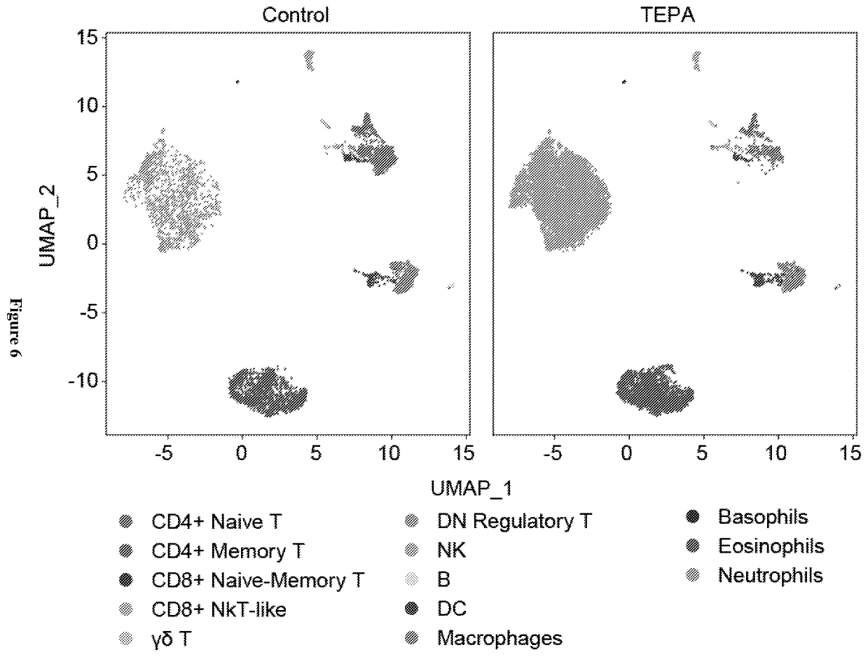

Single cell RNA sequencing was used to assess changes occurring in different cells comprising the tumour microenvironment. Tumour cells were observed to have reduced intracellular copper levels as indicated by expression of associated surrogate genes Mt1 and Mt2. In addition, copper chelation reduced Mycn expression, the key oncogenic driver of neuroblastoma often associated with high-risk disease (FIGS. 4A, 4B, and 5).

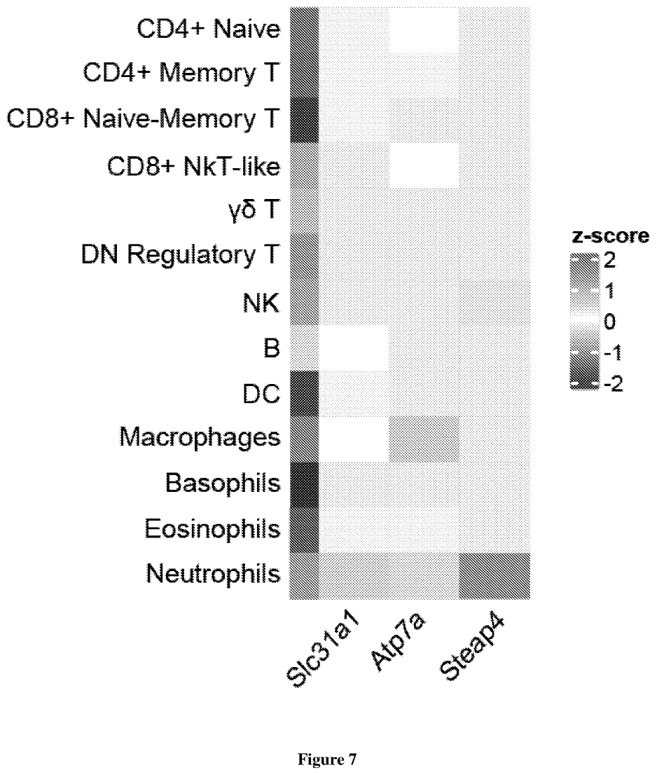

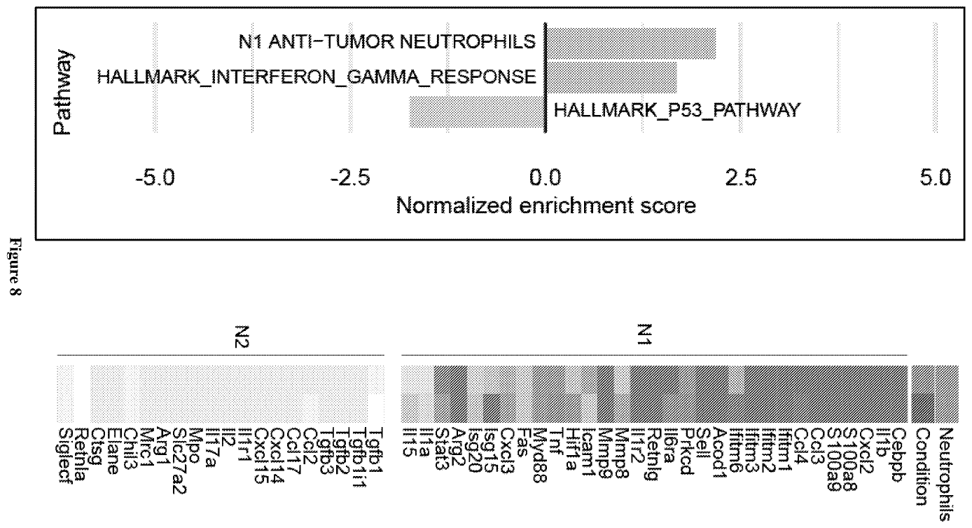

Single cell RNA sequencing of infiltrating immune cells revealed a diverse repertoire of subsets. Importantly, copper chelation induced a near five-fold increase in neutrophil (the most abundant leukocyte type in circulation) infiltration into the tumour microenvironment. Interestingly, the neutrophil subset featured the highest average expression of key genes involved in intracellular copper uptake including Steap4, Slc31a1, and Atp7a (FIGS. 6 and 7). Neutropenia is closely linked to copper deficiency in humans, and can be reversed with oral copper supplementation.

GSEA analysis demonstrated that copper chelation polarised infiltrating neutrophils towards an “N1” phenotype which is strongly associated with pro-inflammatory/anti-tumour effector functions (FIG. 8).

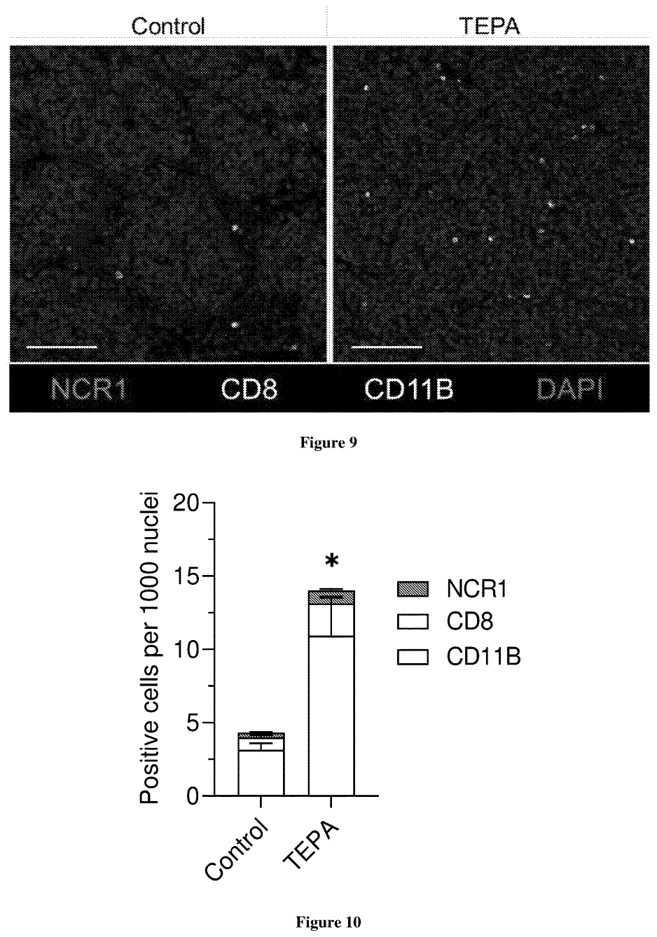

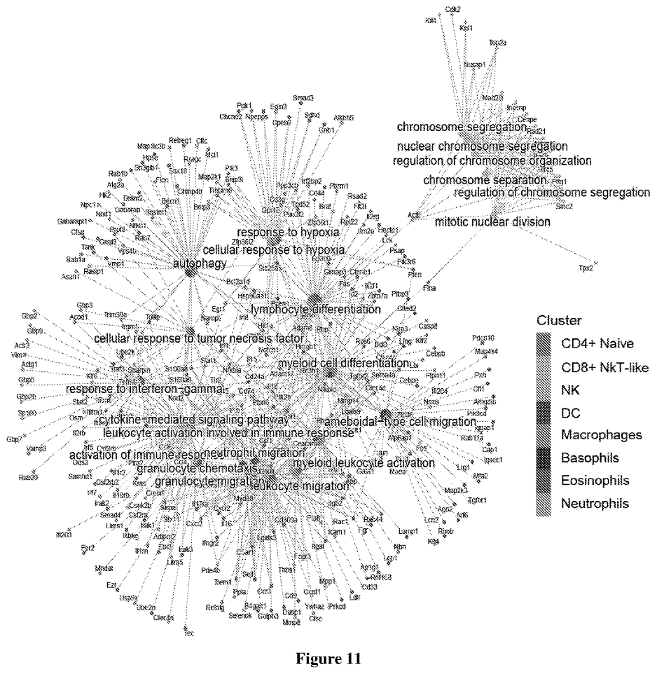

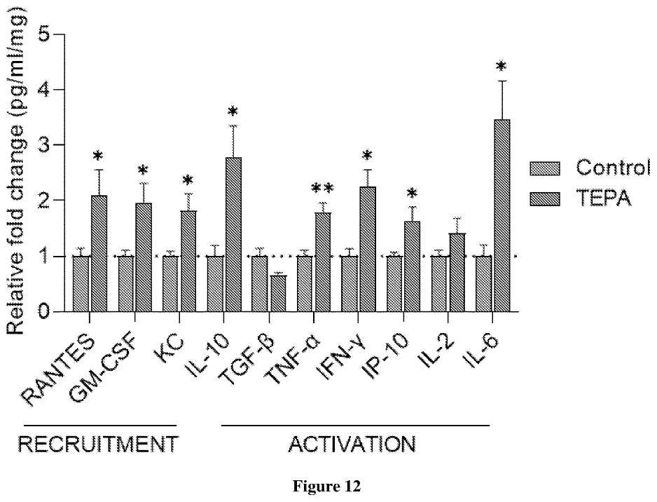

Opal multiplex analyses reflect increased infiltration of CD11B+ myeloid cells in addition to supporting increased NCR1 positive natural killer cell and CD8 positive cytotoxic T cells into the tumour microenvironment with copper chelation (FIGS. 9 and 10).

The single cell RNAseq network analysis reveals key pathways upregulated with copper chelation treatment. Of note, these are associated with lymphocyte and myeloid chemotaxis, activation & differentiation. These are complemented by key associated cytokines which promote recruitment and activation of cells into the tumour microenvironment. Importantly, NK and myeloid cells are associated with mediating antibody-dependent cytotoxicity.

N1-polarized neutrophils have a pro-inflammatory phenotype characterized among others by a higher level of intercellular adhesion molecule (ICAM)-1 and high secretion of interferon (IFN)γ-induced protein 10 (IP-10)/C-X-C motif chemokine 10 (CXCL10) and tumor necrosis factor (TNF). Further, neutrophils incubated under a tumor-mimicking in vitro environment show a high cell surface expression of C-X-C motif chemokine receptor 2 (CXCR2) and secrete high levels of interleukin (IL)-8.

Without being bound by theory, the inventors postulate that copper chelation upregulates lymphocytes, myeloid migration and activation to facilitate an anti-tumour immune response including increased inflammation, functional enrichment of interferon gamma and beta pathways in the TME, increased leukocyte migration to the site of inflammation, lymphocytes and myeloid differentiation and expansion, and the formation of ROS to kill the tumour.

### Example 2: TEPA Transferring Copper From Tumour Cells to Immune Cells

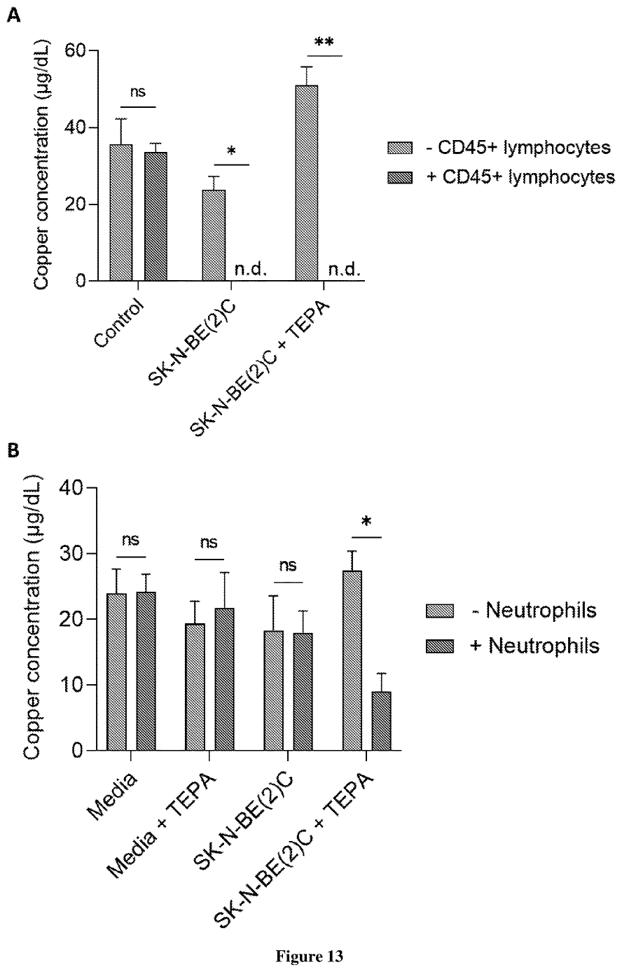

Using an in vitro model of tumour cells (SK-N-BE(2)C)/CD45 positive immune cell co-culture, it was found that TEPA was able to facilitate the release of copper from the tumour cells into the media (FIG. 13A—grey bars). When generic (CD45+) lymphocytes were isolated from whole blood and added (FIG. 13A—red bars), the released copper was completely absorbed from the environment during the immune cell response and activation of associated effector functions. This demonstrates the sensitivity of the immune system to copper in promoting the anti-tumour immune response.

Given previous findings regarding neutrophils in the tumour microenvironment, these were isolated from whole blood using a neutrophil-specific protocol. Again, the results showed the release of copper into the media for subsequent uptake by neutrophils when treated with TEPA (FIG. 13B).

Moreover, it was found that tumour cells specifically suppressed the activation of neutrophils (SK-N-BE(2)-C tumour cells alone condition) which without being bound by theory, the inventors consider suggests that copper chelation promotes immunomodulatory signals (e.g. immune checkpoint molecules) to induce neutrophil activation.

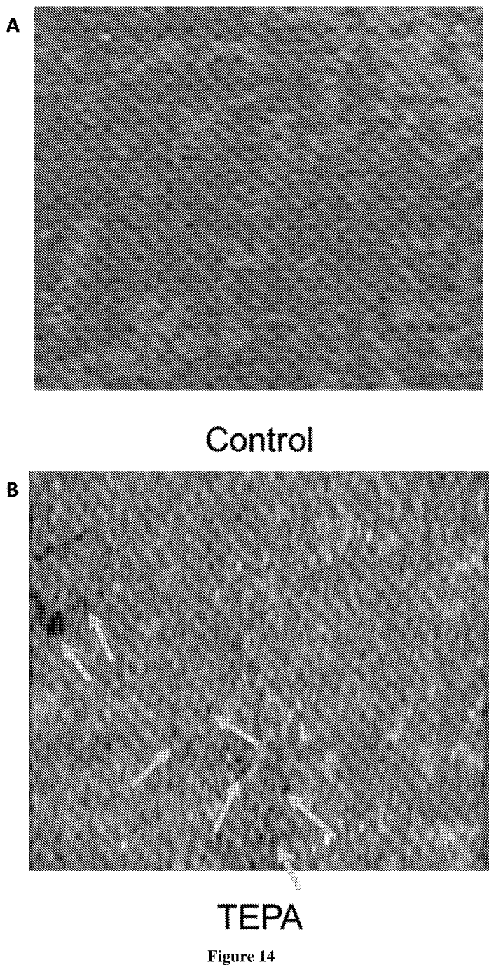

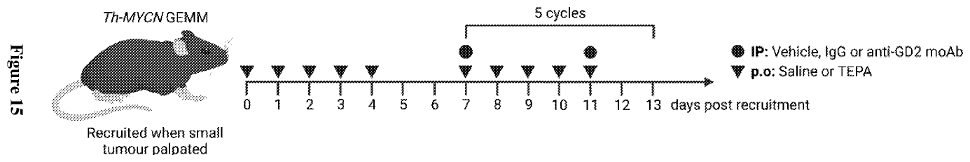

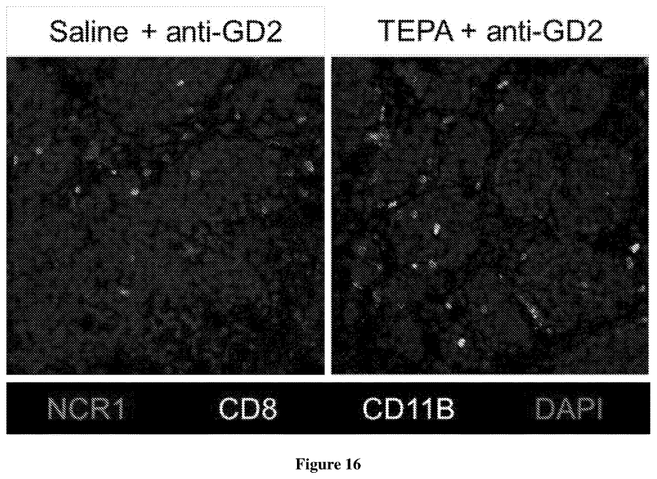

To assess the transfer of copper in situ in mouse neuroblastoma tumours, tissue sections were stained with a copper-specific probe to determine intracellular copper distribution on a single cell basis (FIG. 14). FIG. 14A indicates that TEPA selectively depletes copper from the tumour cells (e.g. control appears overall darker than TEPA) however an intense copper signal is detected in CD45+ infiltrating lymphocytes (black cells indicated by yellow arrows). Collectively, this provides evidence that copper transfers from tumour to immune cells using in vitro and in vivo techniques.

### Example 3: TEPA and Anti-GD2 moAb Combination Therapy in TH-MYCN Transgenic Mouse Model

The gold standard in vivo model of neuroblastoma was used to evaluate the efficacy of copper chelation therapy to enhance anti-GD2 efficacy. TEPA was dosed orally to TH-MYCN GEMM transgenic mice at a daily dose of 400 mg/kg according to the schedule depicted in FIG. 15 (i.e. at days 0, 2, 3, 4, 5, 7, 8, 9, 10, 11, 14, 15, 16, 17, 18 etc. to complete 5 weekly dosing cycles). Mice groups not receiving TEPA received an equivalent volume oral saline dose at the same time points. The mice groups also received either a saline vehicle, IgG, or anti-GD2 ip (20 ug) dose on days 7 and 11 (also repeated over 5 weekly cycles as shown in FIG. 15).

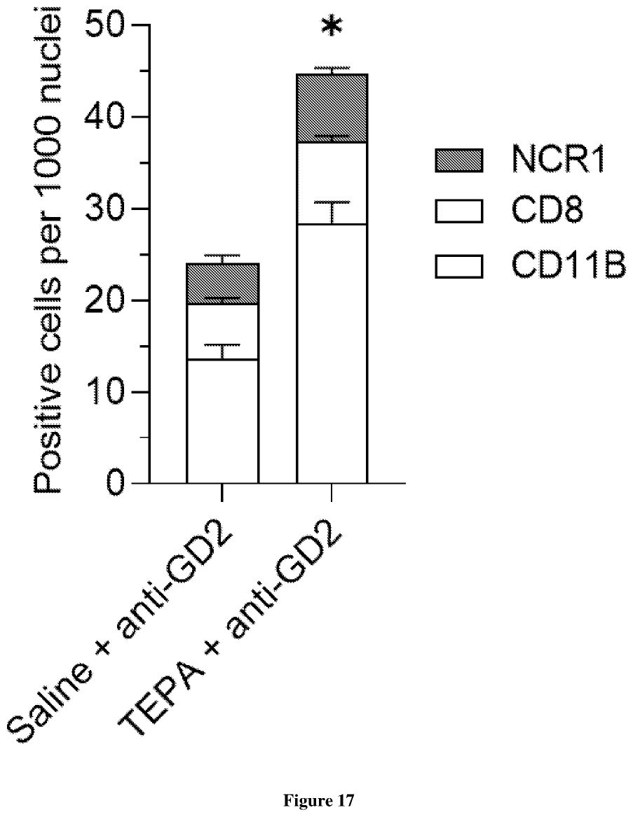

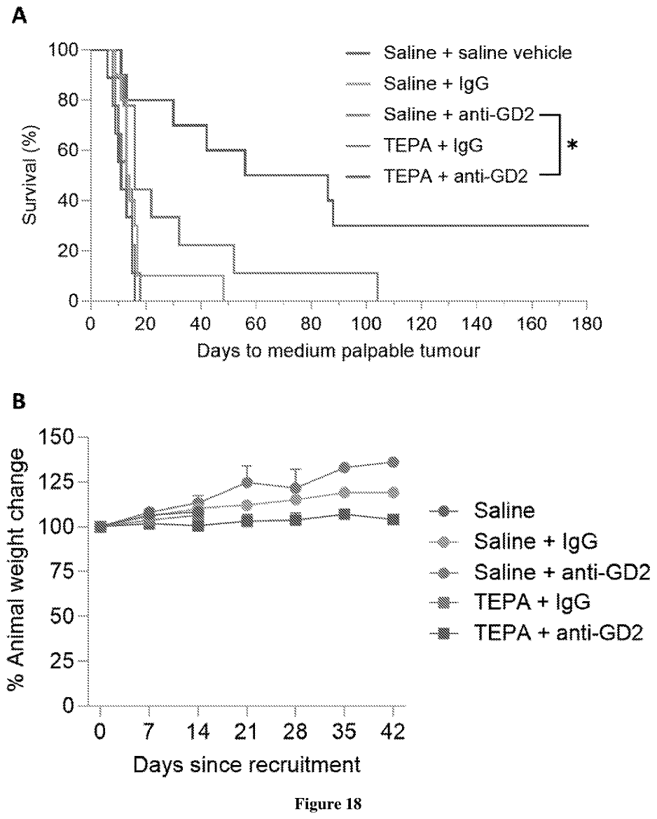

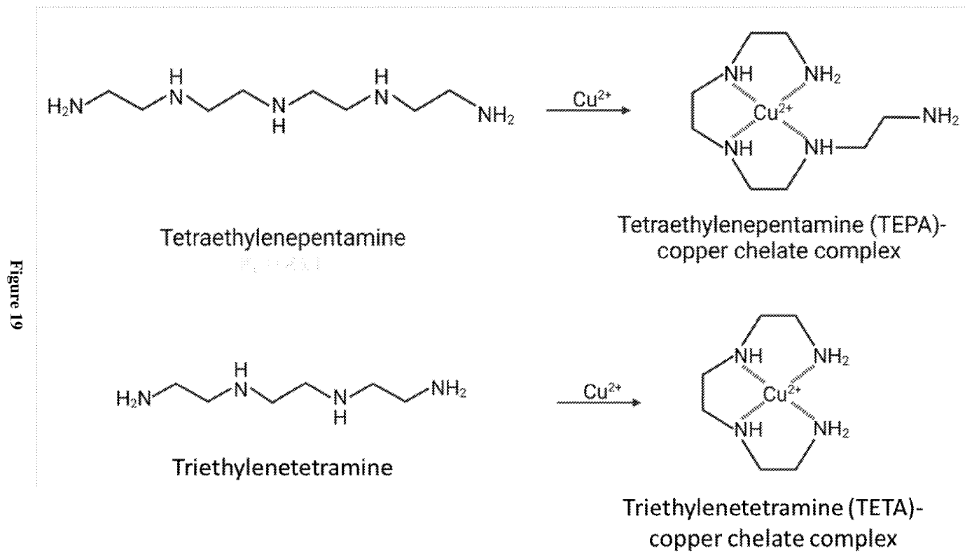

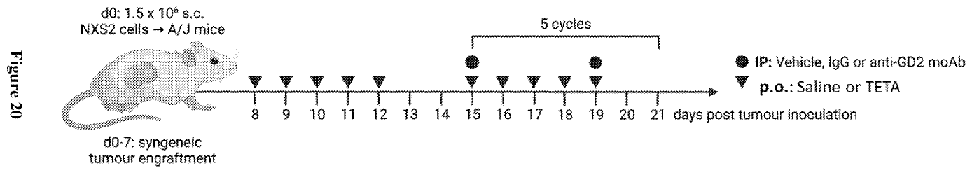

Copper chelation therapy was used to prime the tumour microenvironment to facilitate infiltration and activation of key cell subsets before addition of combination therapy (TEPA+anti-GD2). In a short-term study, Opal multiplex immunohistochemistry showed increased CD8/NCR1/CD11B infiltration when TEPA was combined with anti-GD2 therapy (FIGS. 16 and 17). Furthermore, the combination of this therapy was observed to significantly enhance survival and prevent relapse of disease compared to anti-GD2 alone (FIG. 18A). Copper depletion therapy with TEPA was evidenced to be non-toxic as evidenced by no change in animal weight loss (FIG. 18B). This synergistic effect of the combination of the copper chelator TEPA and anticancer immunotherapy agent anti-GD2 was unexpected, and highlights the effect of copper chelation in preventing immunosuppression by tumours.

### Example 4: TETA and Anti-GD2 moAb Combination Therapy in TH-MYCN Transgenic Mouse Model

Given such promising results with TEPA, TETA was also analysed to see if this effect could be replicated with a different copper chelator. TETA is a clinically approved analogue of TEPA (see FIG. 19), which was first approved for copper overload disorder Wilson's Disease. It is taken orally thrice daily and exhibits an extremely safe toxicity profile in both children and adults despite being used in a life-long treatment schedule.

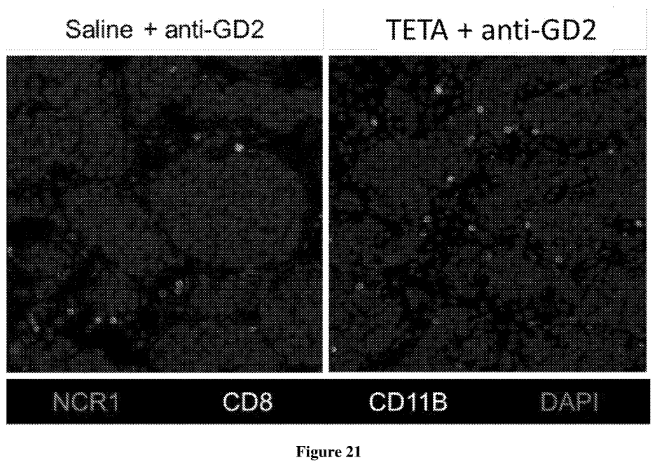

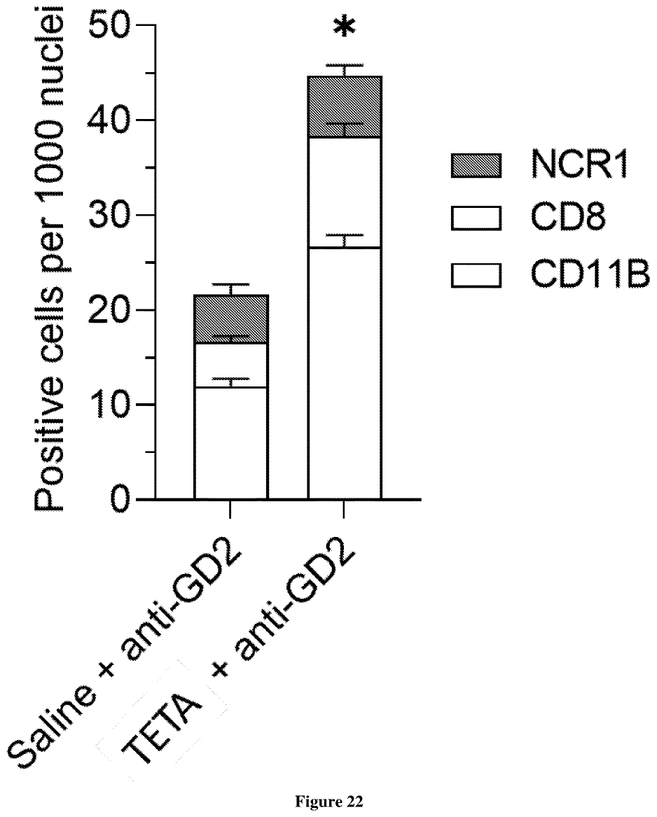

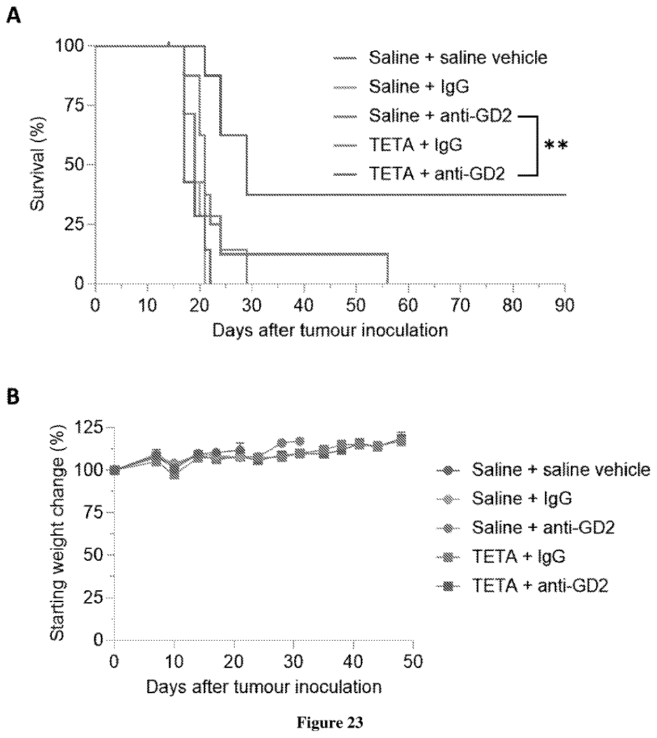

Using a secondary in vivo model of neuroblastoma, the copper chelation agent TETA was similarly trialled according to the dosage regime depicted in FIG. 20. Similar to TEPA, the use of TETA increased CD8/NCR1/CD11B infiltration into the tumour microenvironment using OPAL multiplex immunohistochemistry (FIGS. 21 and 22). Again, it was found that priming the TME with copper chelation therapy was successfully able to potentiate anti-GD2 therapy and prevent relapse in ~30% of animals (FIG. 23A). Importantly, no significant changes to animals were observed with any treatment schedule and underscores the safety profile of this therapy (FIG. 23B).

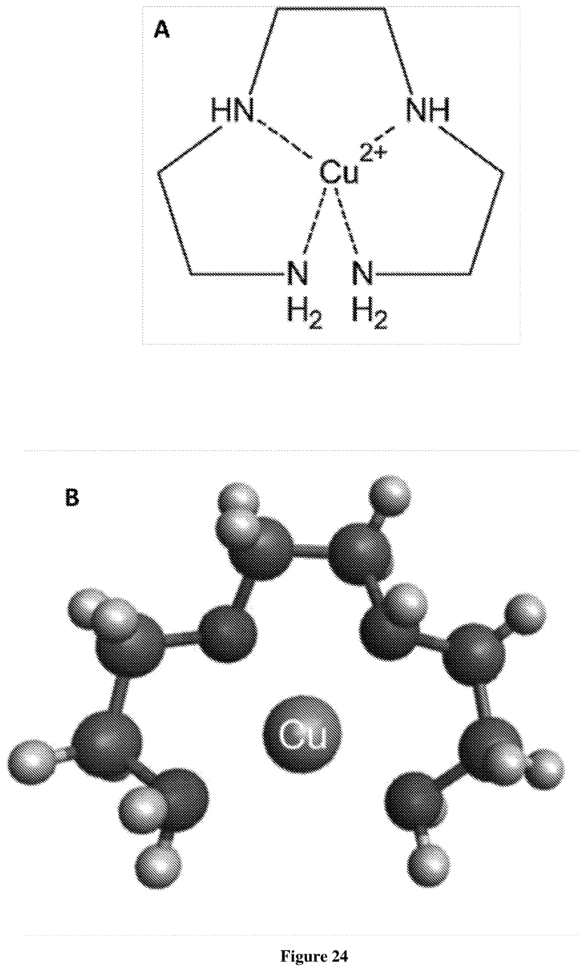

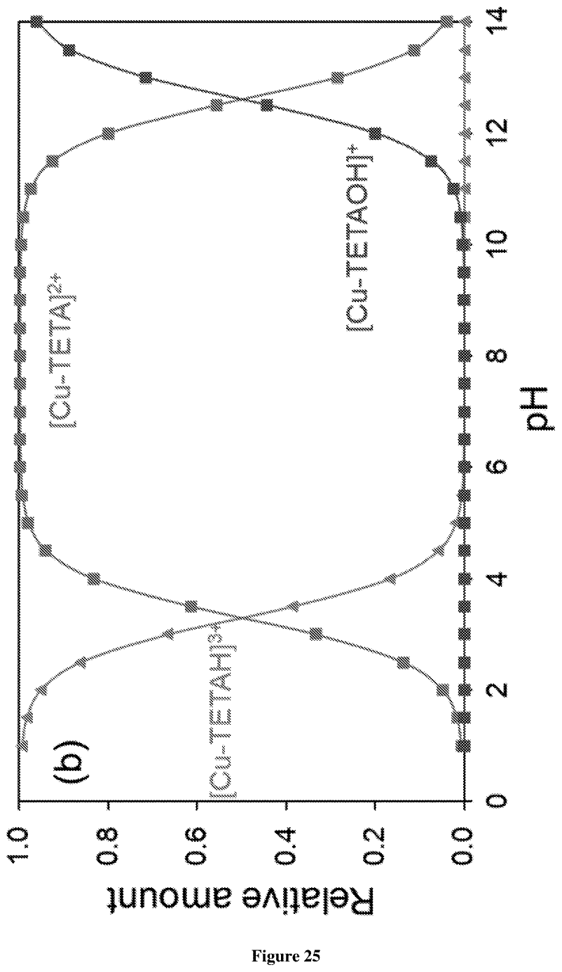

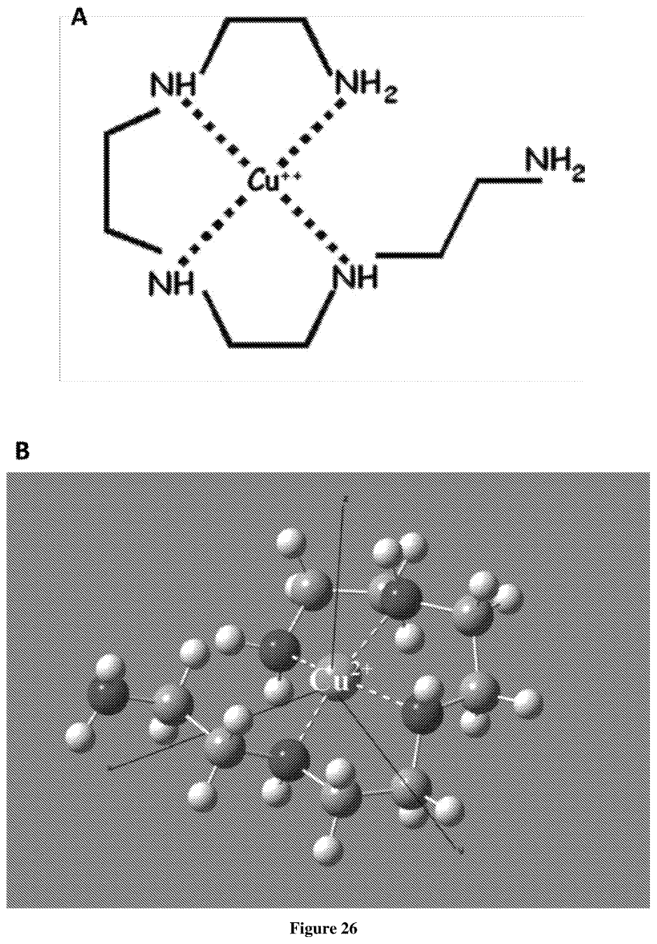

Without being bound by theory, the inventors postulate that these synergistic effects with the combination treatments of either TEPA and anti-GD2 mAb, or TETA and anti-GD2 mAb may follow the following possible mechanism:
Diffusion of TETA/TEPA within cancer cells due to a concentration gradient.
Formation of the complex (it is an equilibrium with the formation of the complex being favoured—see FIGS. 24 and 26 for the complex structure).
Diffusion of the Cu(TETA)²⁺/Cu(TEPA)²⁺ complex outside cancer cells due to a concentration gradient (the formation of the Cu(TETA)²⁺/Cu(TEPA)²⁺ complex is favoured at biological pH—see FIG. 25).
Diffusion of the Cu(TETA)²⁺/Cu(TEPA)²⁺ complex within immuno-cells due to a concentration gradient.
Release of Cu²⁺ in immuno-cells as a consequence of shift of the coordination equilibrium due to the use of copper ions by cells pathways (it is in an equilibrium with the dissociation of the complex being favoured).

### Example 5: Copper Chelation Potentiates Anti-GD2 Antibody Therapy

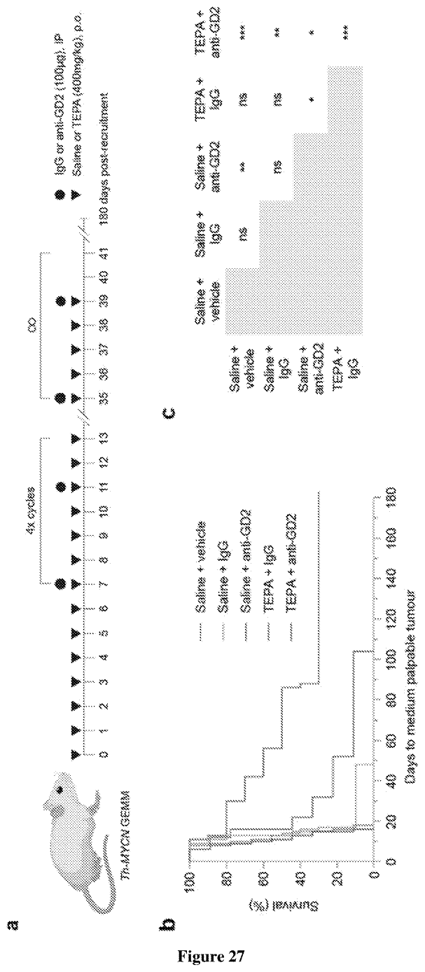

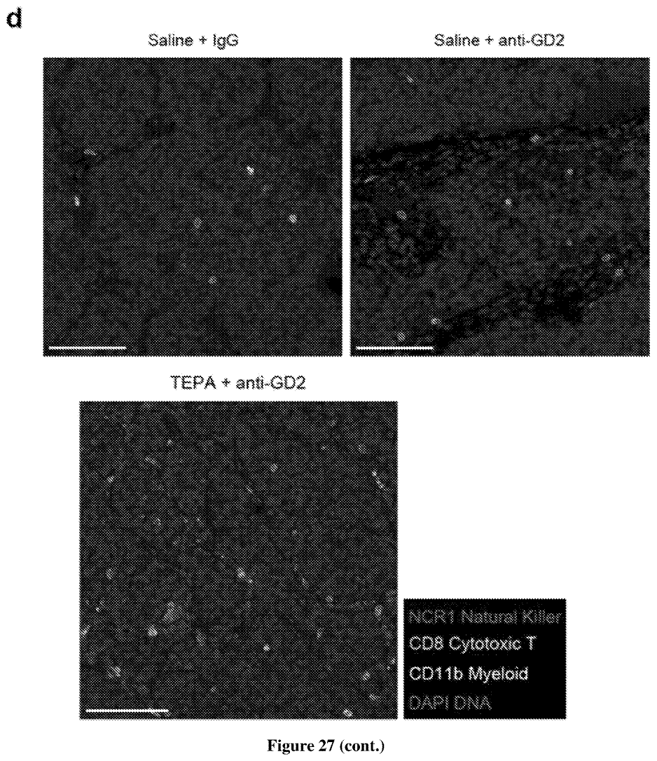

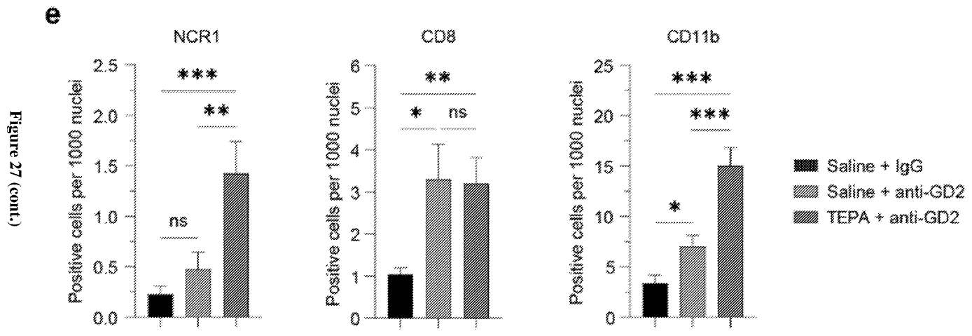

The potential of copper chelation to enhance the efficacy of anti-GD2 antibody therapy in the transgenic Th-MYCN model was tested. As a monotherapy, the copper chelating agent TEPA was administered for one week in mice with established tumors to stimulate immune activation in the tumor microenvironment prior to commencing anti-GD2 antibody therapy. Subsequently, daily treatment with TEPA for four weeks was combined with anti-GD2 antibody therapy twice a week (FIG. 27a). TEPA and anti-GD2 therapy were then reduced to twice weekly administrations until the ethical endpoint (tumor volume≥1000 mm³) or a maximum treatment duration of 180 days was met. Importantly, this immunocombination strategy was well-tolerated with no adverse effects observed.

Anti-GD2 monotherapy induced modest anti-tumor activity, but this was substantially enhanced with the addition of TEPA, resulting in significantly extended survival (p=0.043) and durable responses in ~30% of animals (FIG. 27b,c). To examine associated changes occurring in the immune environment, OPAL multiplex immunohistochemistry was performed on tumors obtained from animals on day 14 post-treatment (FIG. 27d). Anti-GD2 therapy alone significantly increased cytotoxic T (p=0.023) and myeloid cell (p=0.007) infiltration with a trend towards increased NK infiltration compared to the control isotype arm. Nonetheless, the combination of copper chelation with anti-GD2 therapy substantially enhanced the infiltration of NK (p=0.007) and myeloid (p=0.001) cell subsets without impacting cytotoxic T cell infiltration (FIG. 27e). NK cells are recognized as the primary effectors of ADCC in neuroblastoma, eliciting responses through Fc-receptor binding. Unexpectedly, it was observed that copper chelation therapy also increased the frequency of infiltrating CD11b+ myeloid cells which have been associated with immunosuppressive activity in neuroblastoma however can also be engaged as potent effectors of ADCC. This immune-induction strategy demonstrates that copper chelation is an effective adjuvant to increase immune infiltration and enhance tumor control in combination with anti-GD2 antibody therapy.

### Example 6: Copper Chelation Modulates Cytokine Levels to Drive Immune Cell Infiltration

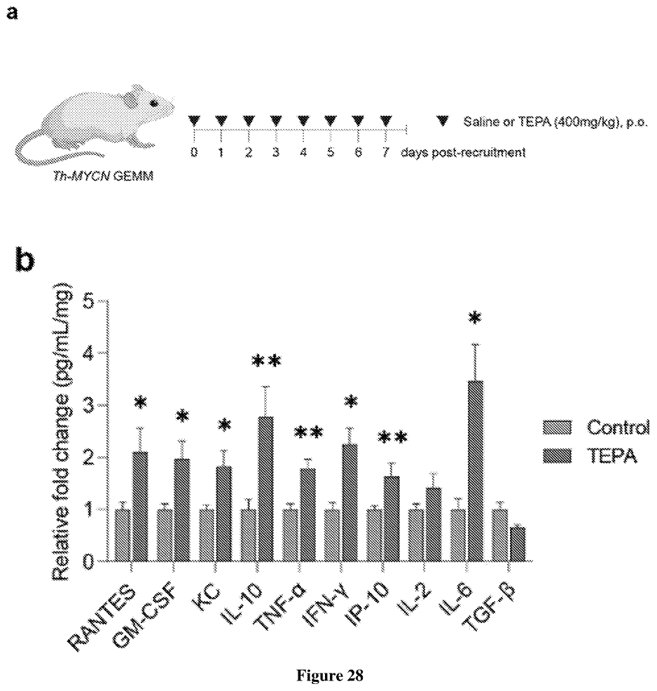

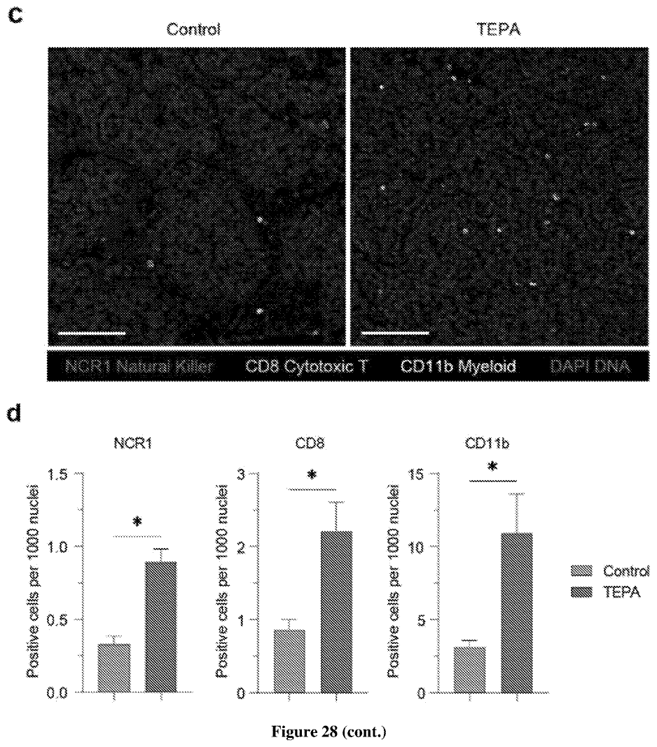

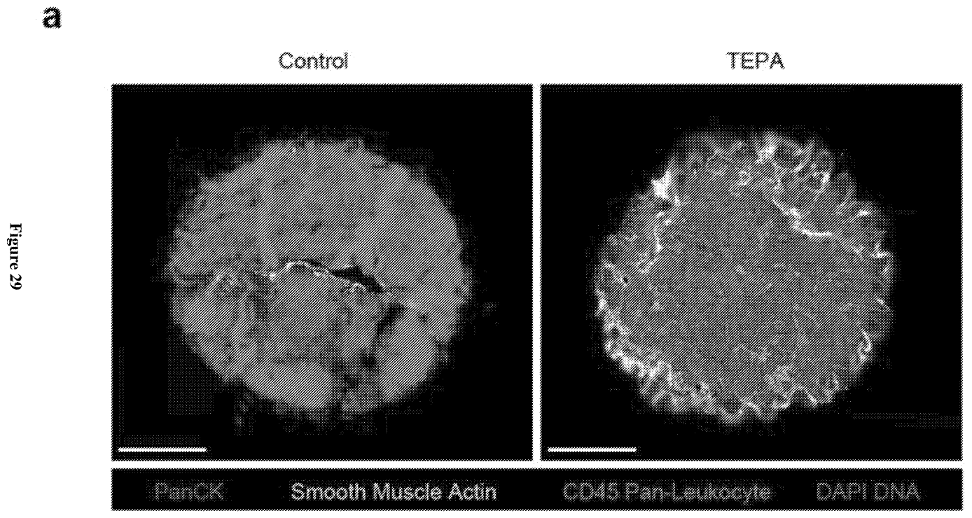

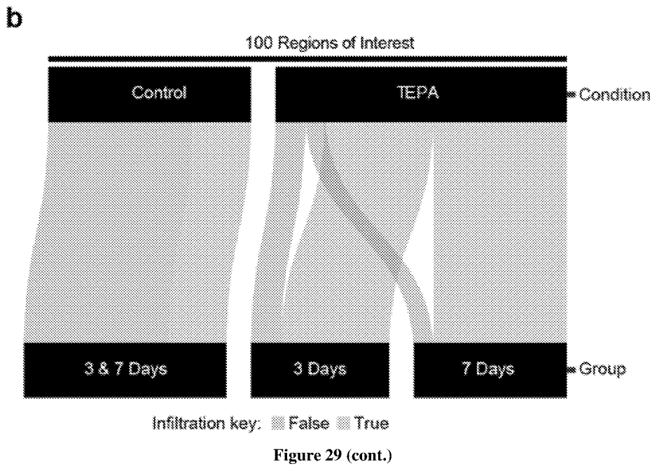

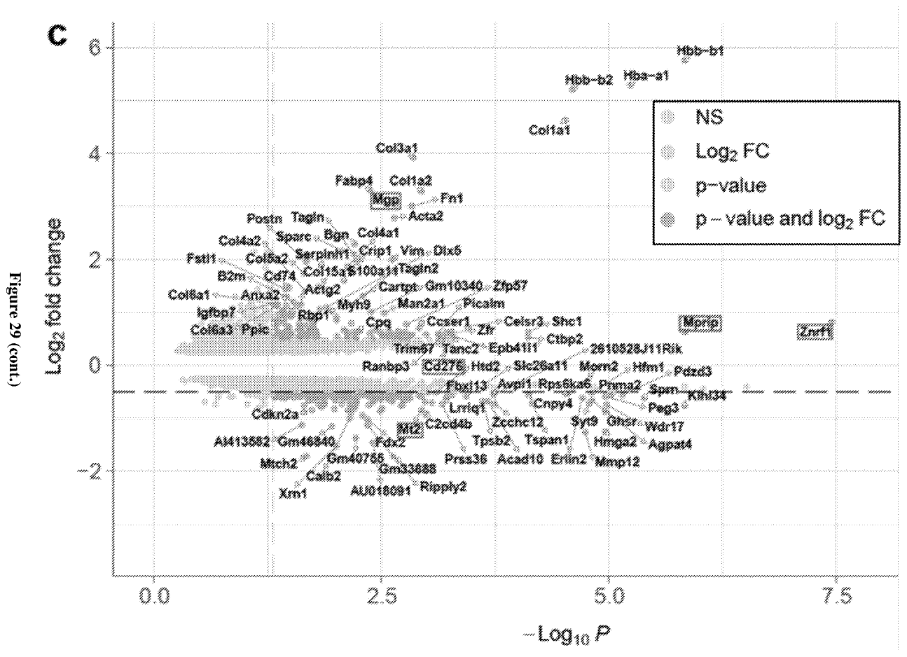

Soluble cytokines secreted in the tumor microenvironment induce pleiotropic effects on immune cells. To understand how the immune response is modulated during the induction sequence, changes occurring within the local cytokine milieu with copper chelation were investigated using multiplex cytokine profiling. Corroborating transcriptomic data, treatment generated significant increase in cytokine levels associated with immune cell recruitment (RANTES/CCL5, GM-CSF, KC/CXCL1/IL-8) and activation and effector functions (IL-10, TNF-α, IFN-γ, IP-10/CXCL10, IL-2, IL-6), while reducing levels of the immunosuppressive cytokine TGF-β (FIG. 28b).

NK cells are recognised as major sources of the above cytokines given their role in promoting anti-tumor responses through regulating effector activity of monocytes, macrophages, neutrophils, dendritic cells, and T cells. Therefore, OPAL multiplex immunohistochemistry was performed to simultaneously validate the presence of NCR1+ NK cells, CD8+ cytotoxic T cells, and CD11b+ myeloid cells within the tumor microenvironment (FIG. 28c). Significant increases in NK and cytotoxic T cell frequencies (FIG. 28d) were observed. Interestingly, copper chelation therapy produced a marked increase in myeloid cells which are known to augment CD8+ T cell effector function in neuroblastoma.

This data suggests that copper chelation favorably modulates cytokine levels to facilitate immune cell infiltration, thereby promoting an immune-permissive neuroblastoma tumor microenvironment.

### Example 7: Copper Chelation Destabilizes the Neuroblastoma Tumor Microenvironment

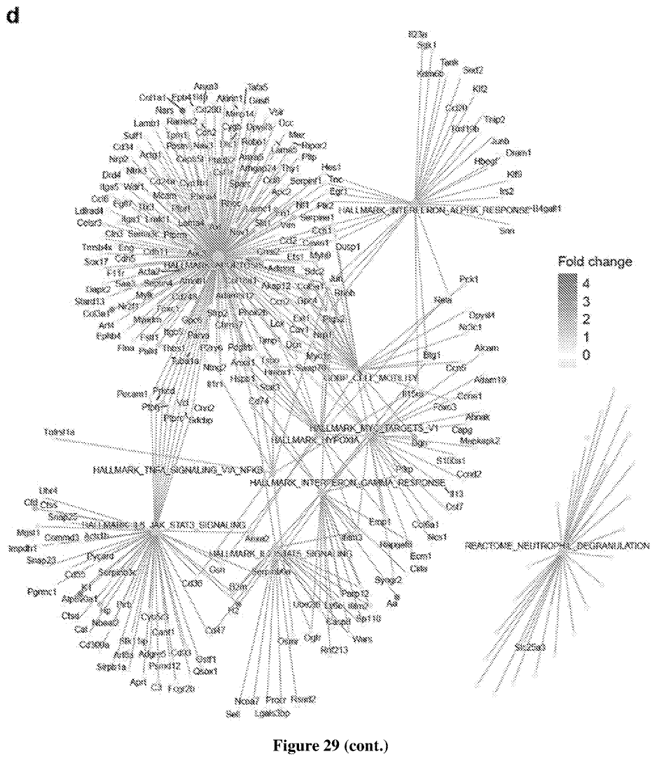

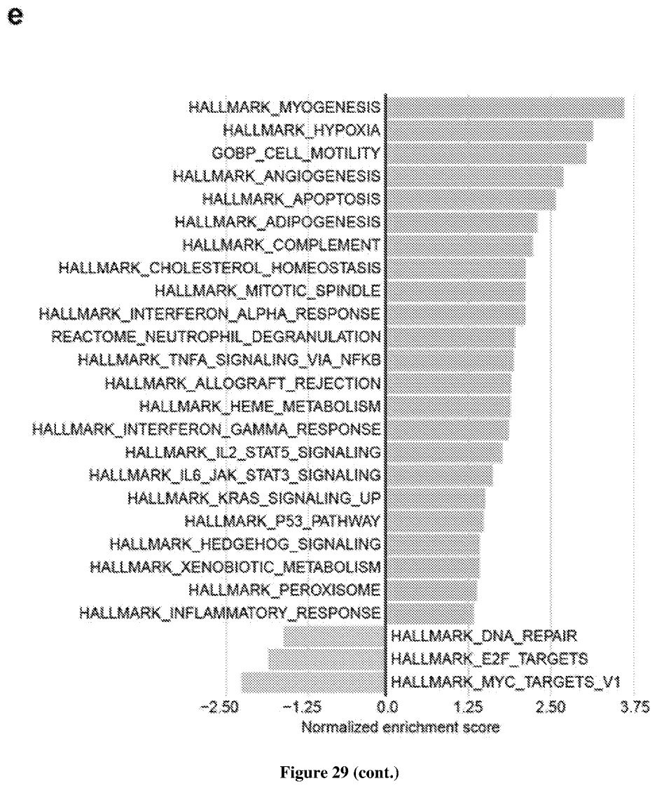

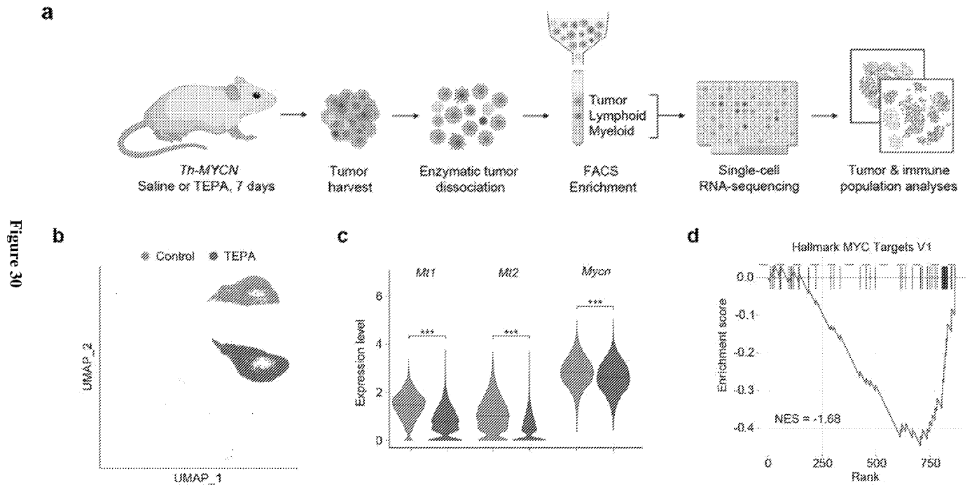

To further characterise changes occurring during induction in the tumor microenvironment, a tissue microarray of Th-MYCN tumors was constructed, consisting of untreated control and TEPA-treated tumors resected after three and seven days of treatment. Utilizing the GeoMx NanoString spatial transcriptomics platform, regions of interest (ROIs) were selected using a ratio of pancytokeratin (PanCK) and pan-leukocyte marker CD45 to assign conditional assignment of false/true immune infiltration for each region. It was observed that copper chelation therapy with TEPA was strongly associated with immune infiltration, as determined by CD45 staining of tumor cores (FIG. 29a). When comparing false/true infiltration assignments, TEPA-treated tumors were observed to exhibit a time-dependent increase in immune infiltration (FIG. 29b). Next, differential gene expression analysis was performed, comparing TEPA-treated infiltrated versus TEPA-treated non-infiltrated ROIs to minimize cross-talk between tumor and immune cell signals. Interestingly, the upregulation of Mgp and Mprip together with Znrf1 was observed, which is suggestive of neuronal differentiation, a key feature associated with a favorable clinical prognosis in neuroblastoma (FIG. 29c). Gene set enrichment analysis reports upregulation of pro-inflammatory pathways involved in interferon (IFN) and tumor necrosis factor (TNF) signaling, the p53 pathway involved in tumor suppression and downregulation of MYC targets responsible for oncogenic signaling (FIG. 29d,e). Taken together, these results strongly indicate a destabilization of the neuroblastoma tumour microenvironment.

### Example 8: Copper Chelation Enhances Neutrophil Infiltration in the Neuroblastoma Tumor Microenvironment

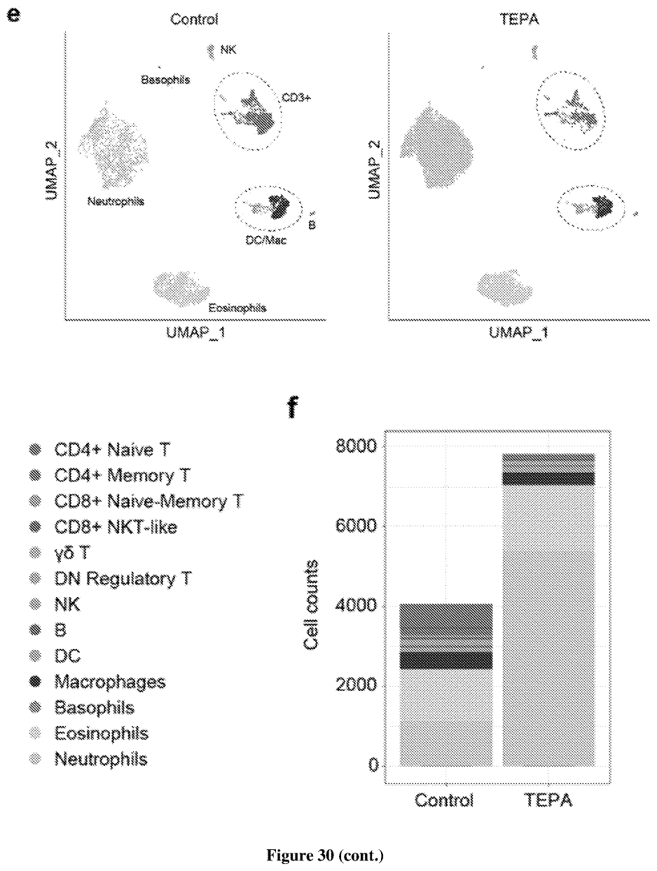

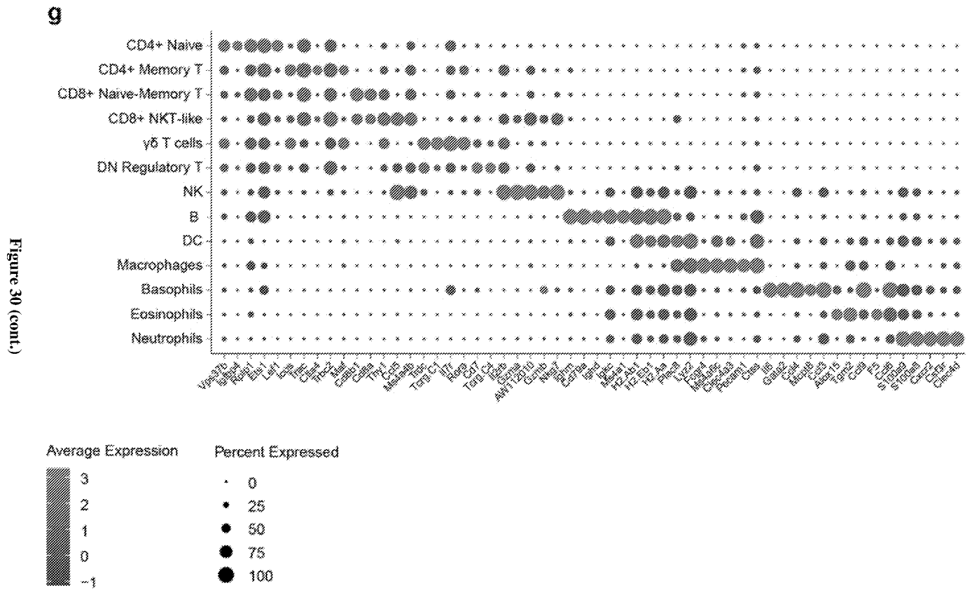

To characterize functional changes in cells a single-cell resolution, tumors treated for seven days with saline or copper chelating agent TEPA using BD Rhapsody system were analyzed. Fresh tumor sections were dissociated using a protocol that ensured high viability of both tumor and immune compartments for subsequent enrichment by flow cytometric sorting (FIG. 30a). After quality control, dimension reduction, and clustering, 13,555 tumor cells (defined using Mycn gene expression; FIG. 30b) were obtained. Importantly, tumor cells were highly responsive to copper chelation therapy with a significant decrease in gene expression of the metallothioneins Mt1 and Mt2, surrogate markers for intracellular copper concentration (FIG. 4c) (21). Unexpectedly, the tumoral oncogene Mycn and associated targets were also significantly downregulated which has been reported to decrease tumor cell proliferation, increase immunogenicity and increase immune cell infiltration (FIG. 30c,d).

The immune compartment consisted of 12,127 immune cells (defined using Ptprc [CD45] gene expression; FIG. 30e) with a diverse repertoire of 13 unique clusters identified in both treatment arms (FIG. 30e-g), including lymphoid and myeloid lineages and unconventional subsets such as CD8+ NKT-like and gamma-delta (γδ) T cells. This data supports tumor-associated immune subsets in the Th-MYCN model and clinical neuroblastoma samples as reviewed by others; however proportions may vary owing to different analytical techniques and tumor stages. The number of cell clusters remained largely unaltered by copper chelation treatment with the notable exception of neutrophils which exhibited a profound five-fold increase after treatment (FIG. 30f). To assign this cluster, the recently identified gene markers S100a8/a9 were used, which together form the heterodimer calprotectin involved in neutrophil recruitment and activation alongside previously established markers (FIG. 30g).

Collectively, this data demonstrates the impact of copper chelation in the neuroblastoma tumor microenvironment, including tumor cell downregulation of Mycn and its targets and increased neutrophil infiltration.

### Example 9: Copper Chelation Reinvigorates the Anti-Tumor Immune Response Via Neutrophil Signaling

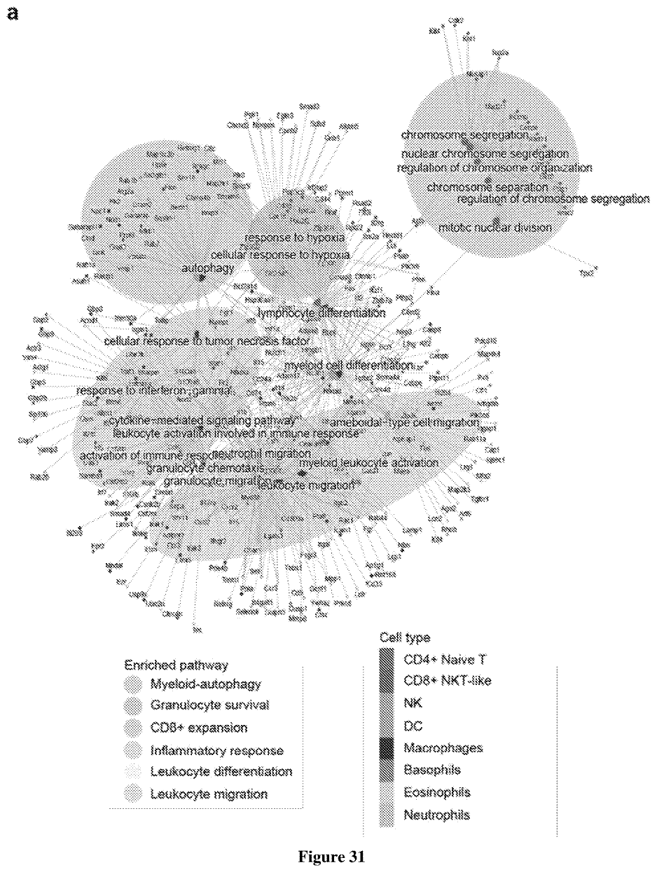

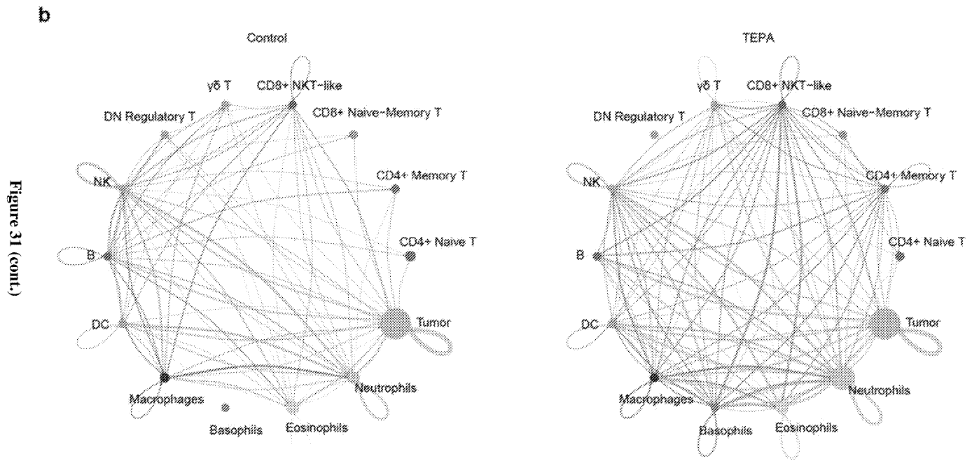

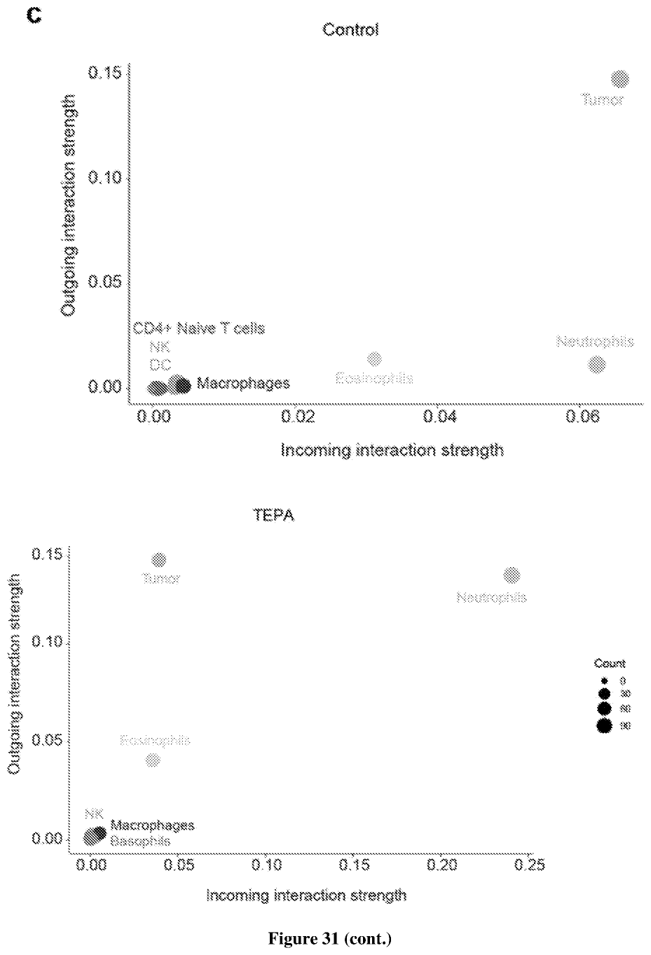

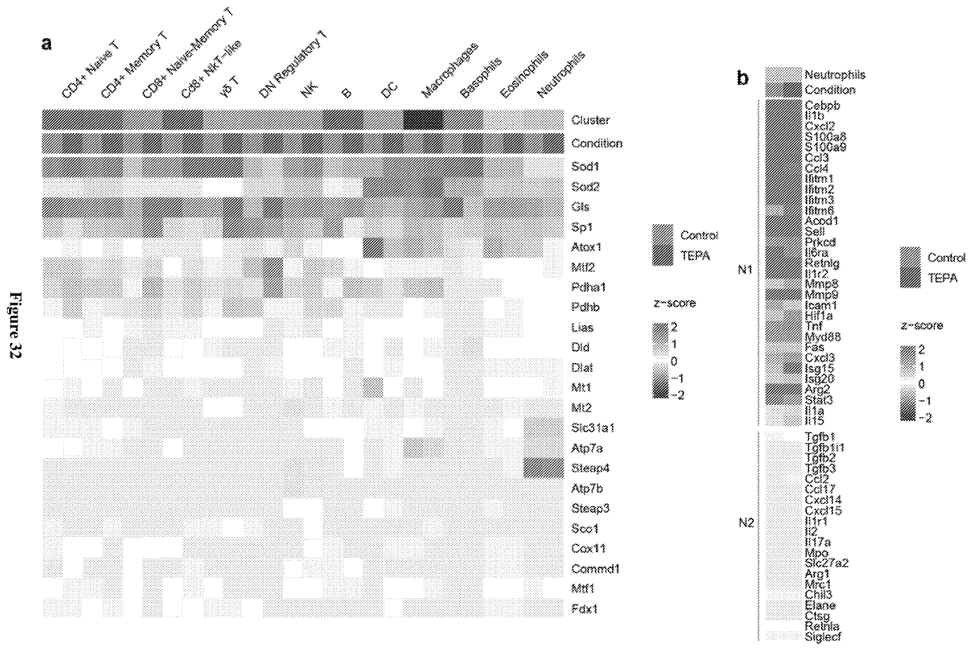

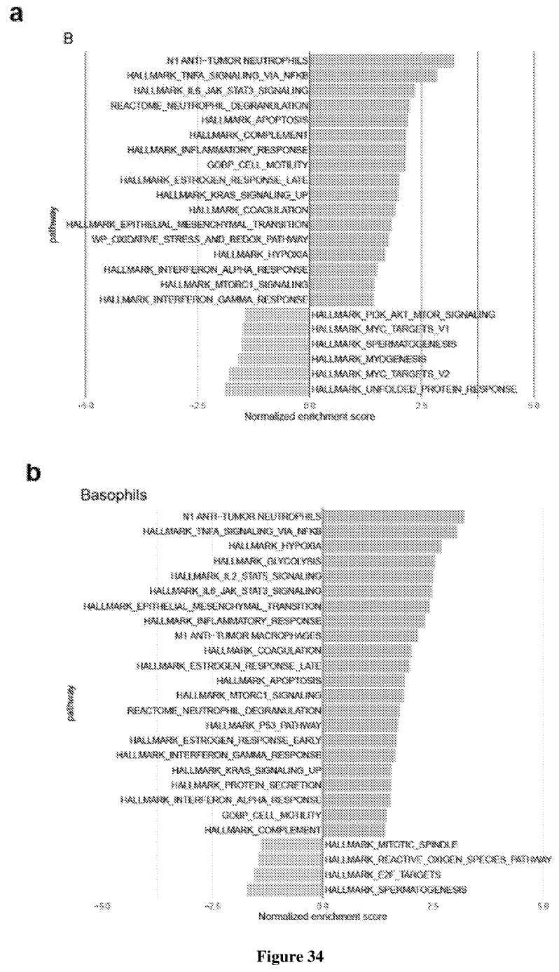

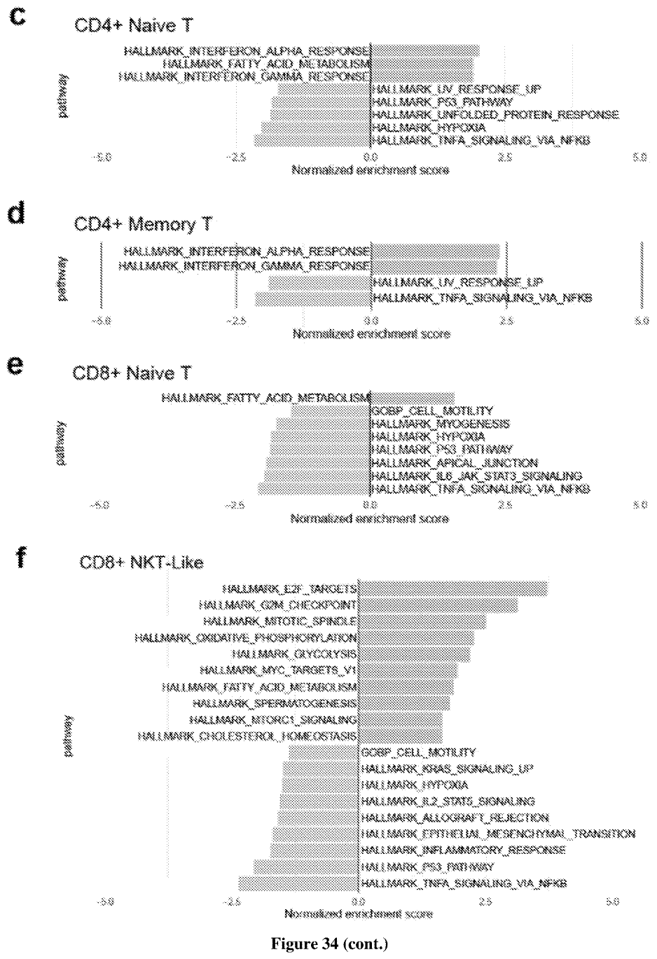

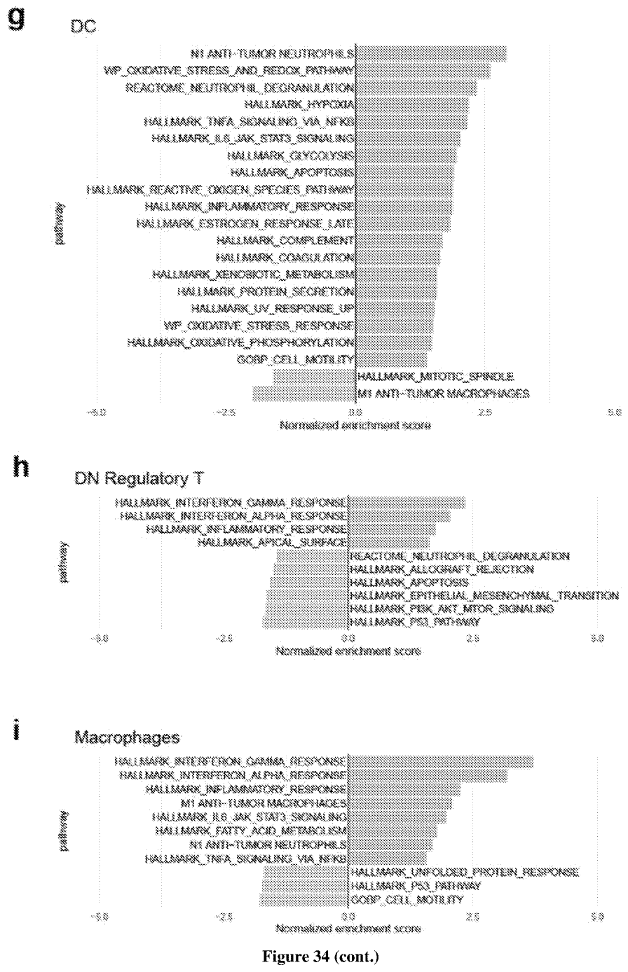

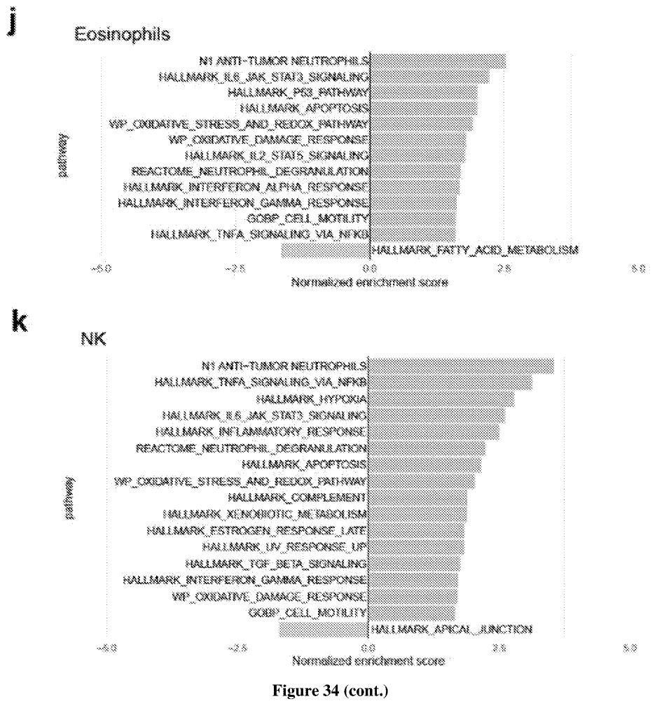

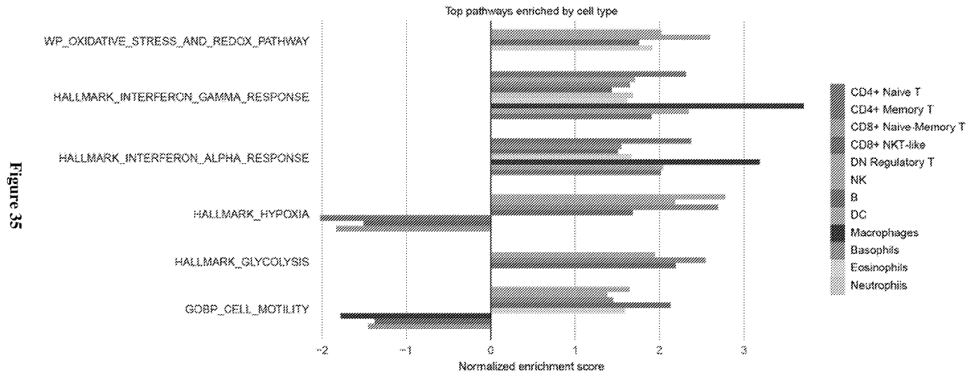

Next, the impact of copper chelation on the immune compartment was assessed by mapping biological processes in the treatment and control arms. Enrichment was observed for gene sets related to CD8+ expansion, myeloid-associated hypoxia and autophagy responses, lymphocyte and myeloid migration, differentiation, and activation as well as cellular cytokine responses to IFN-γ and the tumor necrosis factor (TNF) family (FIG. 31a). Bar plots and top pathways produced by gene set enrichment analysis for each immune cluster upon TEPA treatment indicate metabolic alterations associated with activation of the innate immune response (FIGS. 34 and 35).

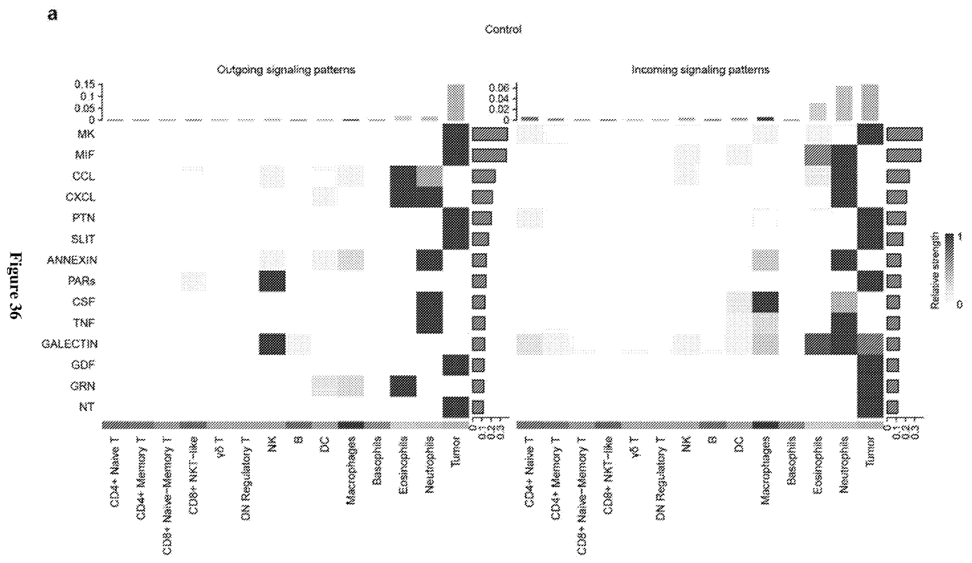

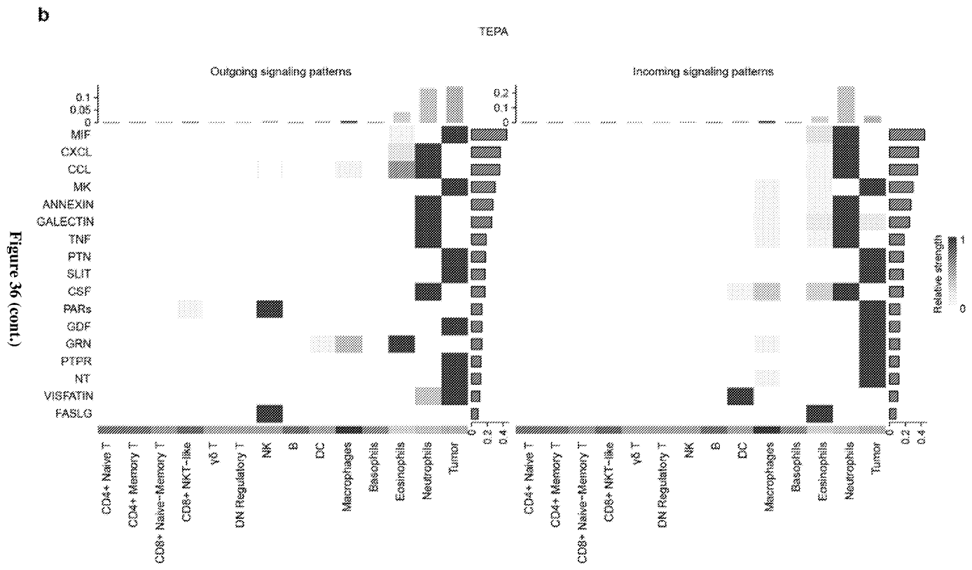

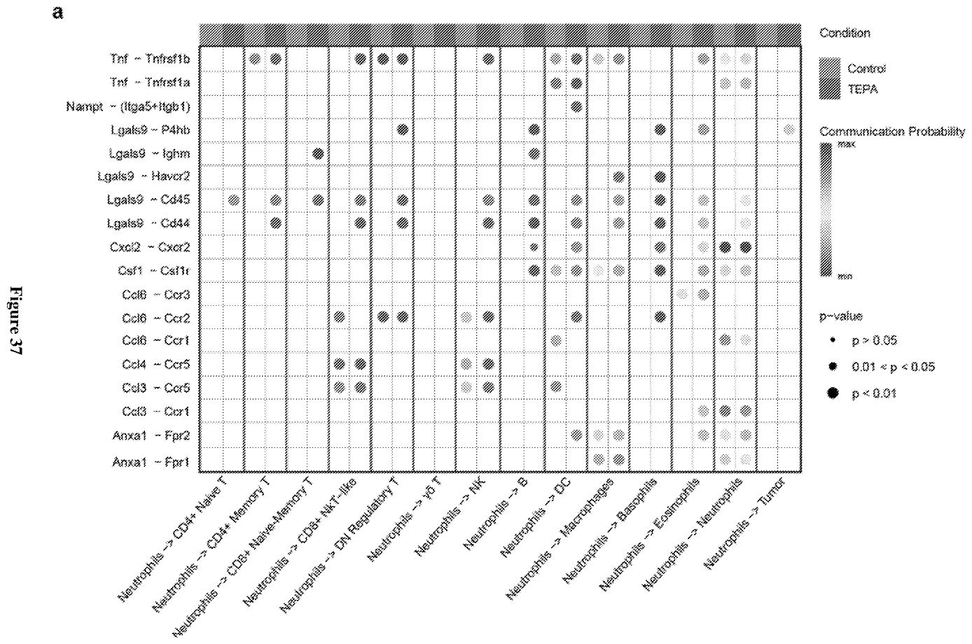

The anti-tumor immune response involves complex coordination of multiple cell types across both innate and adaptive cell subsets. To understand how these processes may be facilitated, CellChatDB was used to infer changes in cell-cell communication between immune and tumor clusters in control and TEPA-treated datasets. An overall increase in signaling networks occurring with copper chelation was observed, dictated by the presence and or increased strength of cell-cell interactions (FIG. 31b). Furthermore, treatment caused neutrophils to supersede tumor cells when comparing incoming interaction strengths, alluding to dampening of tumoral-induced immunosuppression (FIG. 31c; FIG. 36). Given the increase in infiltrating neutrophils with TEPA treatment, ligand-receptor expression occurring between this cluster and other subtypes was examined, and it was found that this was largely driven by the Galectin-9 (Lgals9) axis (FIG. 37). Without being bound by theory, the inventors posit that copper chelation may skew the immunosuppressive tumor microenvironment and reinvigorate the anti-tumor response via enhanced neutrophil signalling.

### Example 10: Mobilization of Copper Enhances Anti-Tumor Activity of Neutrophils

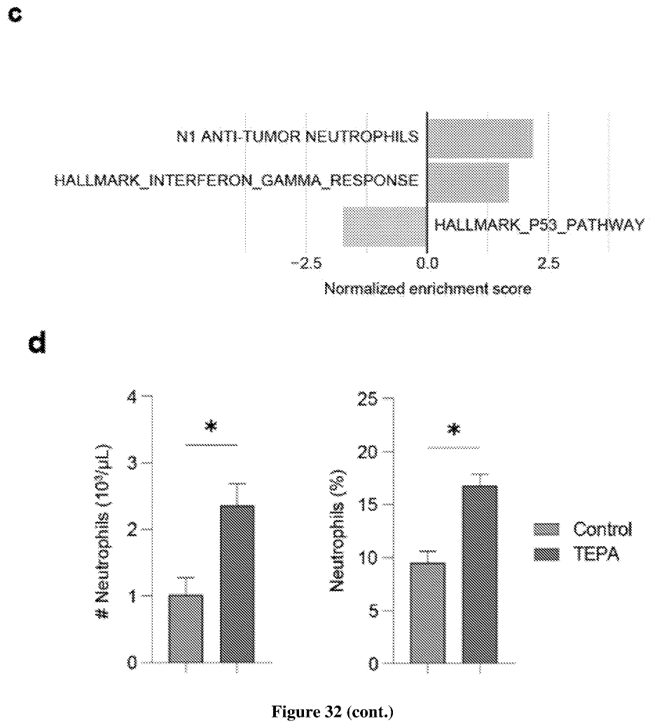

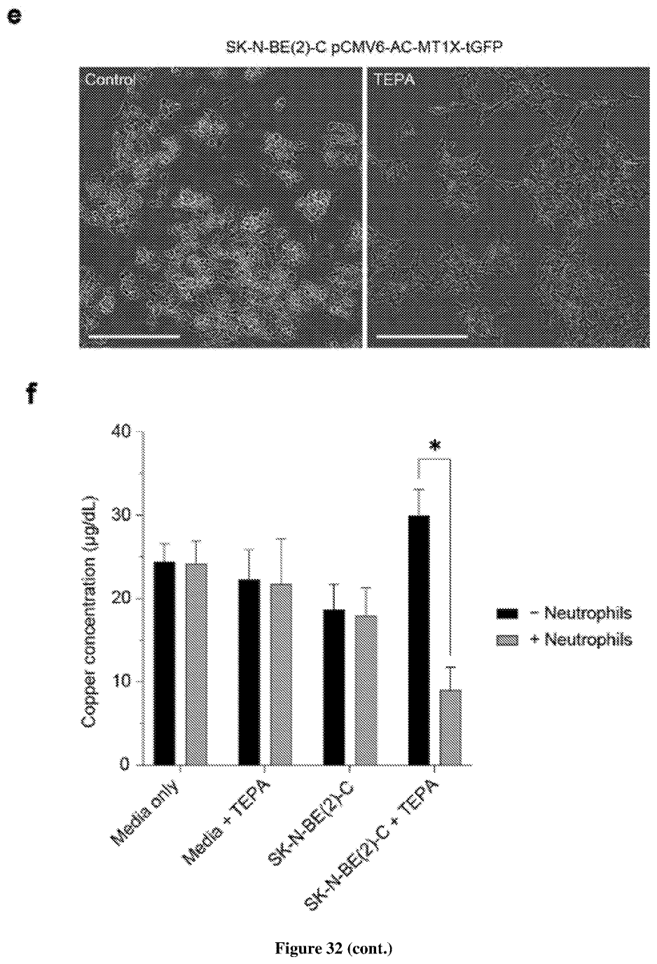

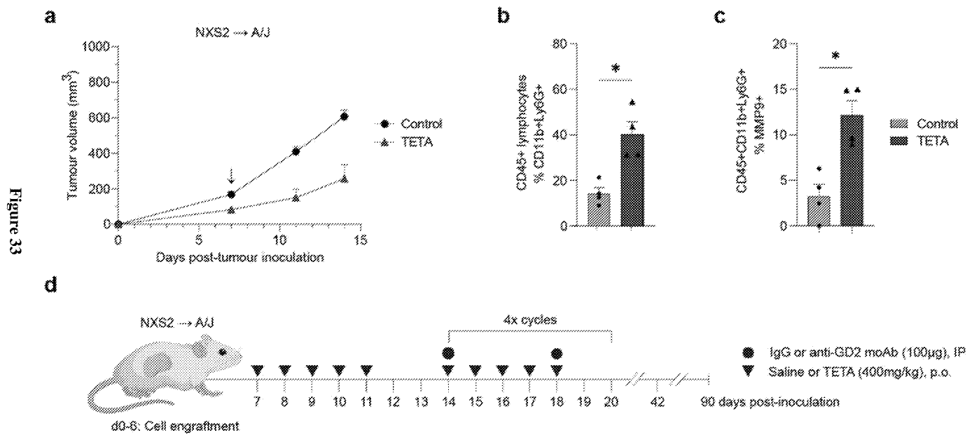

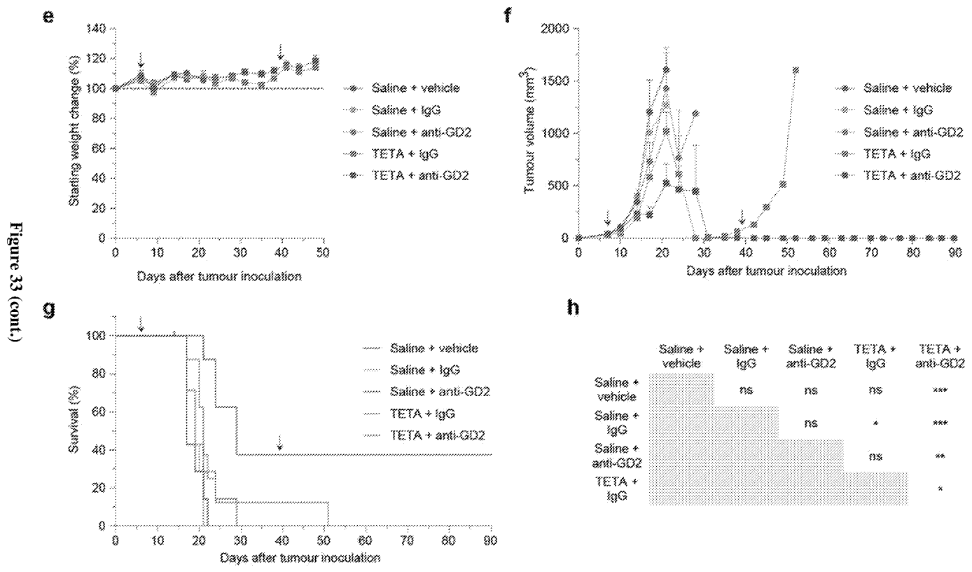

Given the critical importance of copper for effective immune function, changes in copper metabolism occurring within the immune compartment were investigated using single-cell transcriptomics data. Comparing treatment groups, changes in the selected set of copper-related genes was not observed, which may be attributed to the tight homeostatic regulation of copper in non-malignant, healthy cells (FIG. 32a). However, neutrophil-restricted expression of Slc31a1, Atp7a and Steap4, the major genes responsible for intracellular copper import and export in mammals, were observed.

Mirroring the M1-M2 spectrum used for macrophage polarisation states, N1-N2 has recently emerged for neutrophils for respective assignment of anti- and pro-tumor functions. Using recently published datasets, an N1 and N2 gene signature was curated and it was found that tumor-infiltrating neutrophils exhibited a pro-inflammatory N1 phenotype independent of copper chelation treatment (FIG. 32b). A gene set enrichment analysis comparing treatment arms was then performed, and a significant enrichment in genes associated with the curated N1 anti-tumor neutrophil and IFN-γ response pathways was observed (FIG. 32c). IFN-γ stimulation has been demonstrated to enhance N1-associated properties such as ICAM1 expression, reactive oxygen species and neutrophil extracellular trap formation, direct and antibody-dependent cellular cytotoxicity, as well as T cell recruitment and activation.

As the cytokines GM-CSF and KC are potent neutrophil chemoattractants upregulated by copper chelation therapy (FIG. 28b), the inventors postulated that copper chelation may induce neutrophil mobilization from the bone marrow. To assess this, the peripheral blood of control and TEPA-treated animals were immunophenotyped and significant increases in both abundance and relative percentages of circulating neutrophils were confirmed (FIG. 32d). No other immune subsets or erythrocyte measurements were significantly affected (data not shown), with these results collectively underscoring the exclusive relationship between copper levels and neutrophils.

Systemic copper deficiency has been associated with reduced numbers of circulating neutrophils and depressed effector function, which can be rapidly reversed with copper supplementation. As neuroblastoma cells exhibit elevated copper content to drive tumor progression (e.g., proliferation, angiogenesis, metastasis), the inventors postulated that this sequestration may create a copper-depleted microenvironment to suppress immune cell function, particularly neutrophils. The inventors therefore hypothesised that copper chelating agents such as TEPA can redirect the flow of copper ions from the “rich” tumor cells to the “poor” immune cells to slow tumor progression and reinvigorate anti-tumor immunity.

To test this hypothesis, single-cell findings were validated by transducing the neuroblastoma cell line SK-N-BE(2)-C with an MT1X-tGFP construct. This allowed visual confirmation that the fluorescent signal (proportional to MT1X expression, a surrogate marker for intracellular copper) was indeed reduced in cells after 24 hr of TEPA treatment (FIG. 32e). This suggests that, in vitro, tumoral copper is released extracellularly and hence media copper concentration can be assayed to infer copper distribution.

To this end, a staggered co-culture system was devised allowing accurate tracing of copper flow during a simulated neuroblastoma-neutrophil interaction using the SK-N-BE(2)-C cell line. Conditioned media was collected, and copper concentration assayed before and after the addition of naïve circulating neutrophils isolated from healthy donors. Of note, copper concentration was marginally decreased in the presence of media isolated from untreated neuroblastoma cells relative to control media which indicates cells sequester available copper to support their proliferation (FIG. 32f, black bars). In line with the hypothesis, a repletion of copper after tumor cells were treated with TEPA was observed, which was subsequently absorbed by neutrophils after incubation (FIG. 32f, grey bars). Importantly, this phenomenon did not occur in untreated neuroblastoma cells, indicating the existence of secreted soluble factors that impede neutrophil copper uptake, and are restored by copper chelating therapy.

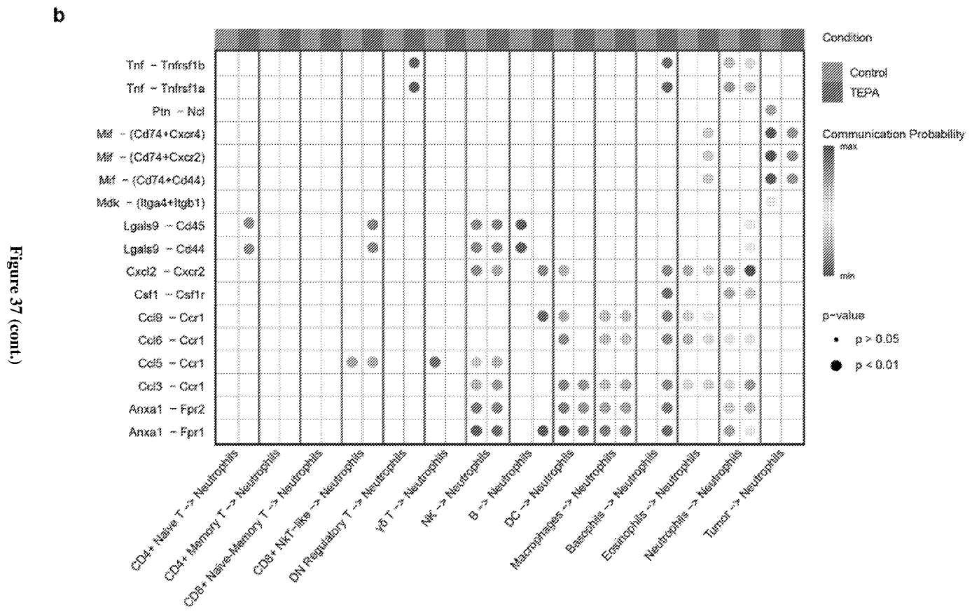

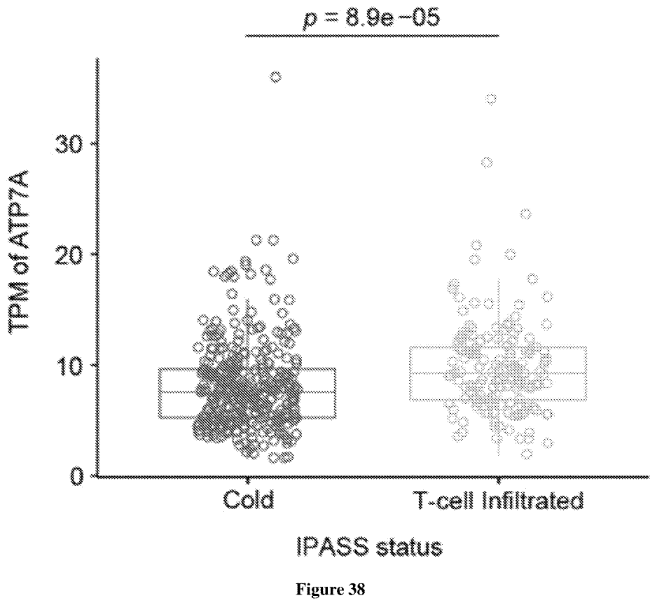

In support of this phenomenon, elevated levels of copper export protein ATP7A was significantly associated with a T cell-infiltrated tumor microenvironment in a wide range of solid pediatric malignancies, including neuroblastoma (FIG. 38). This indicates redistribution of copper within the tumor environment is indeed advantageous and is likely expedited by the use of copper chelating therapy.

These findings demonstrate the effects of copper chelation to stimulate the recruitment of pro-inflammatory neutrophils and enhance N1 functions associated with an anti-tumor response.

### Example 11: TETA Plus Anti-GD2 Antibody Immunocombination Therapy Offers a Curative Strategy for Neuroblastoma

While the Th-MYCN model is considered the standard for the preclinical study of MYCN-amplified neuroblastoma tumors, manual palpation is used to determine tumor burden. To enable better monitoring of tumor growth kinetics, a syngeneic model generated by subcutaneous injection of NXS2 cells (derived from a hybrid of the C1300 neuroblastoma cell line and dorsal root ganglion cells) into immunocompetent A/J mice was utilised. This model has been widely used to study anti-GD2-directed therapies including combination therapies. Having demonstrated the capabilities of copper chelation therapy, the feasibility of repurposing TETA (an analog of TEPA), a copper chelating agent FDA-approved for first-line treatment of Wilson's Disease was evaluated.

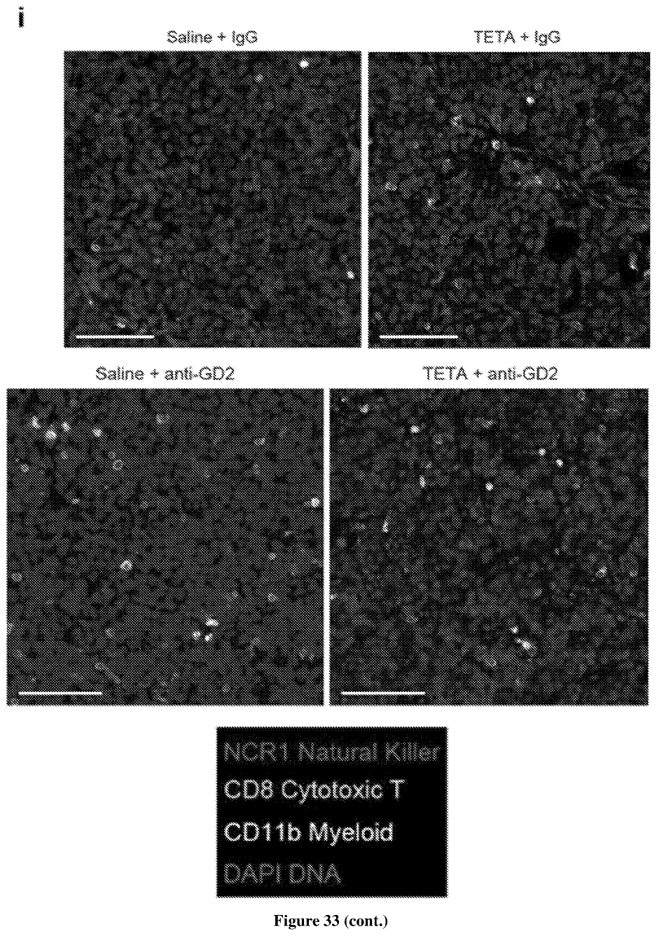

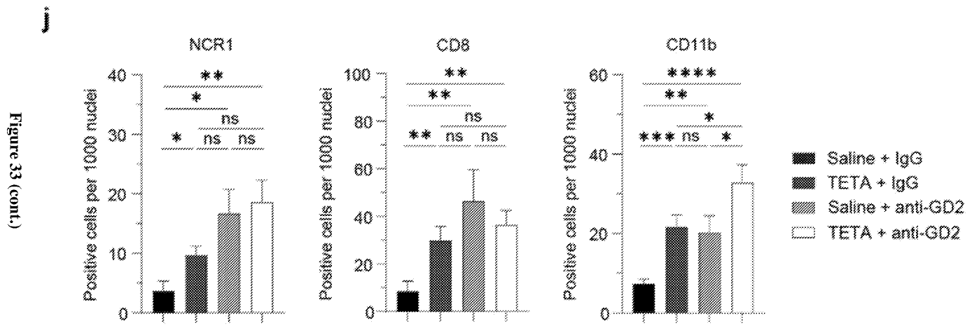

Using the syngeneic NXS2 model, treatment was commenced on day seven post-inoculation (FIG. 33a, black arrow) and animals were randomly assigned to receive control (saline) or TETA (400 mg/kg) daily by oral gavage for seven days. Similarly to TEPA, TETA slowed tumor growth as a single agent (p=0.028, day 14) (FIG. 33a). Subsequently, dissociated tumors were evaluated for neutrophil infiltration and phenotype via flow cytometry. TETA treatment increased frequencies of CD11b+Ly6G+ neutrophils which exhibited increased expression of MMP9, associated with an N1-polarised state (FIG. 33b,c). This indicates that TETA stimulates infiltration and activation of pro-inflammatory N1 neutrophils which are emerging as key effectors of antibody-based immunotherapy.

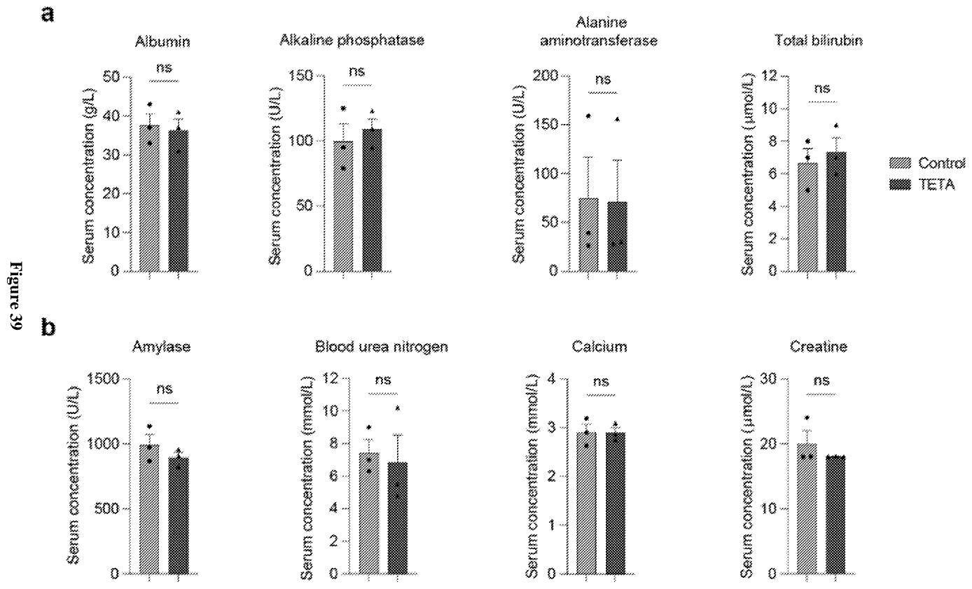

In patients with Wilson's disease, copper chelation therapy can generate bursts of free copper levels which often induce toxic side-effects. To identify potential adverse effects experienced in a tumour-bearing context, peripheral blood was obtained from NXS2-inoculated mice following weeklong TETA treatment and was subjected to blood chemistry analysis. TETA treatment did not impact any analyte concentrations associated with hepatic, renal or overall systemic disorders compared to the control (FIG. 39). This is of particular importance in consideration of the liver as the systemic reservoir of copper.

Overall, these results validate TETA as an alternative to TEPA to favorably remodel the neuroblastoma tumor microenvironment.

This encouraging data prompted investigation of the effect of TETA and anti-GD2 antibody as a combination therapy in the NXS2 syngeneic model. The treatment schedule was similar to that utilised in the TEPA/Th-MYCN model; however, treatment was ceased after four cycles of combination therapy to evaluate relapse rates (FIG. 33d). Upon commencement of the treatment schedule, mice underwent a slight weight loss attributed to the introduction of daily gavage as a stressor. Once accustomed, all treatments were well-tolerated including the immunocombination arm with no adverse events reported (FIG. 33e). As a highly aggressive model of neuroblastoma, control arms exhibited rapid tumor expansion. However, the immunocombination arm was observed to effectively restrain tumor growth (FIG. 33f). It is noted that treatment with TETA+IgG sufficiently reduced tumor burden in a single animal which succumbed to a rapid relapse following cessation of treatment on day 42, potentially due to tumor escape. In contrast to the Th-MYCN model (FIG. 27b,c), anti-GD2 therapy alone did not mediate a substantial anti-tumor effect. Despite this, the TETA+anti-GD2 antibody combination produced remarkable anti-tumor effects (median survival: 29 days vs 19 days in saline+anti-GD2, p=0.009) leading to durable eradication in ~40% of animals (FIG. 33g,h). Notably, these animals did not exhibit any signs of relapse following cessation of treatment up to the experimental endpoint of 90 days. Overall, the addition of TETA to anti-GD2 therapy significantly extended survival when compared to respective monotherapy arms.

To examine changes in the immune compartment (NCR1+ NK cells, CD8+ cytotoxic T cells, and CD11b+ myeloid cells) as a result of treatment, OPAL multiplex immunohistochemistry was performed in tumors resected 14 days post-treatment (FIG. 33i). Control arms (saline+vehicle; saline+IgG) were highly similar in terms of tumor growth and survival which suggests the isotype antibody did not elicit an immunogenic effect. Across all subsets examined, TETA and anti-GD2 monotherapies were comparable and significantly promoted immune infiltration when compared to the control (FIG. 33j). The addition of TETA to anti-GD2 therapy predominantly enhanced CD11b+ infiltration (p=0.02), presumed to be neutrophils, with no observed changes occurring in NCR1+ or CD8+ frequencies (FIG. 33j). Remarkably, the combination group exhibited exceptional tumor control, alluding to TETA-mediated activation of the anti-tumor immune response.

Collectively, these results reinforce the critical role of copper as a modulator of the tumor microenvironment in neuroblastoma. These results demonstrate the ability of copper chelating agents to successfully circumvent immune evasion phenotypes and elicit a robust anti-tumor immune response. Moreover, the results confirm that TETA is a highly effective, non-toxic, and specific copper chelating agent. Study findings provide the first evidence for repurposing the clinically approved copper chelating agent Cuprior® as an immunomodulatory agent to potentiate anti-GD2 immunotherapy and improve responses in patients with neuroblastoma.

### Example 12: Combination Treatment With Copper Chelator TEPA and GD2-Targeting CAR T Cells

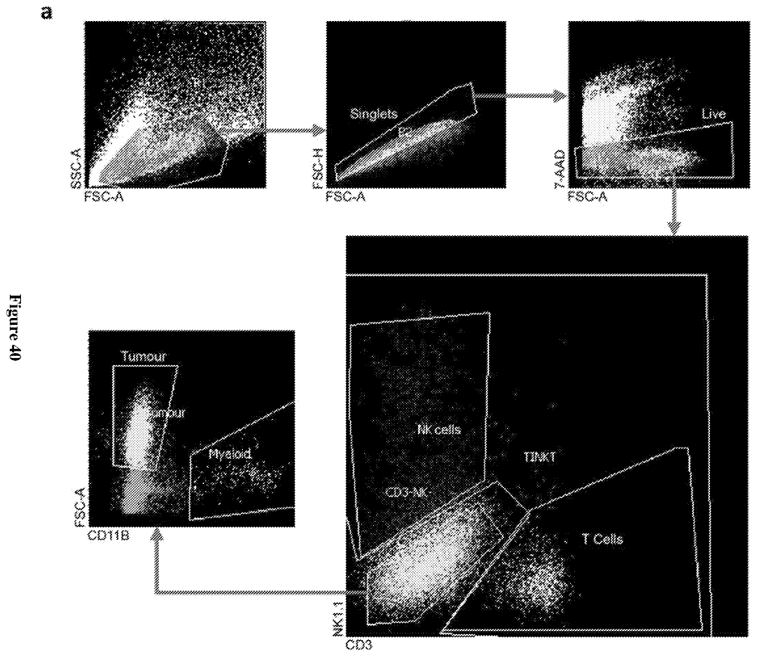

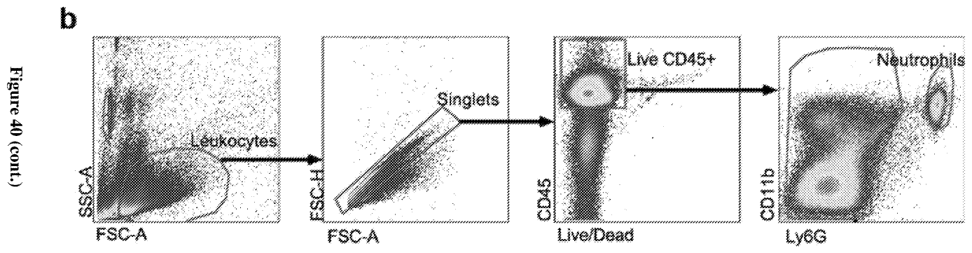

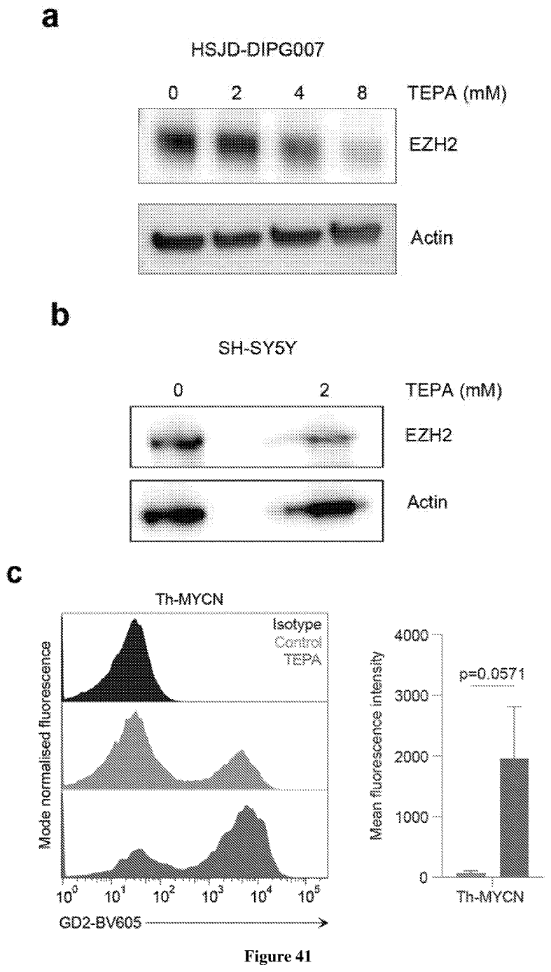

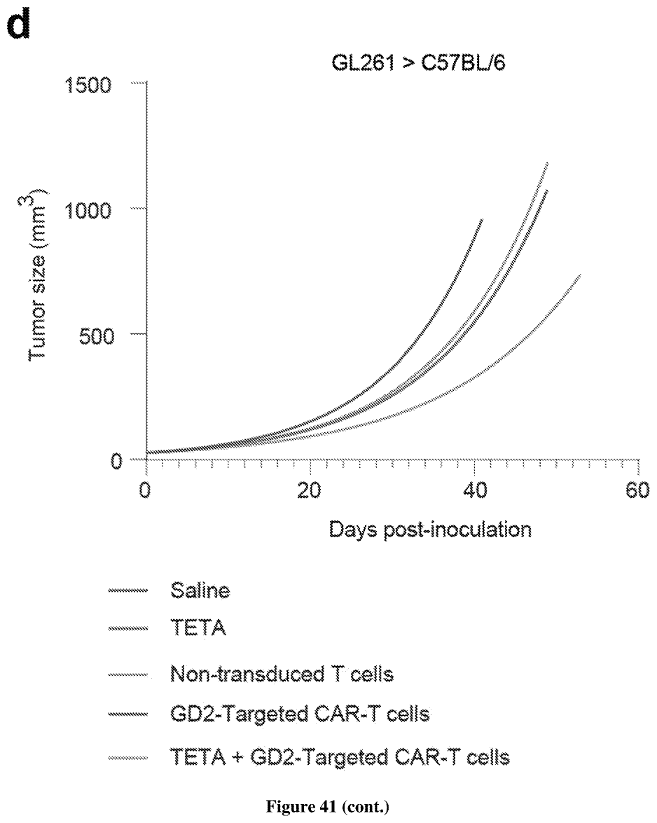

EZH2 inhibition is emerging as an exciting avenue to increase GD2 antigen expression in neuroblastoma and Ewing's sarcoma. It was found that the copper chelator TEPA robustly downregulated EZH2 in diffuse midline glioma (DMG), an aggressive childhood brain cancer (FIG. 41a) and neuroblastoma cell lines (FIG. 41b). Translating this work in vivo, it was found that TEPA robustly upregulated GD2 expression in Th-MYCN neuroblastoma tumours after one week of treatment using flow cytometry (FIG. 41c). Interestingly, preliminary data from a xenograft model of glioblastoma demonstrated that one week of TEPA pre-treatment had a synergistic effect in reducing tumour growth in combination with GD2-targeting CAR T cells (1×10⁶ cells IV in one injection). This data strongly suggests copper chelation can epigenetically modulate GD2 antigen expression and thereby increase sensitivity to GD2 CAR T immunotherapy. Importantly, these results support the idea that by reducing intra-tumoural copper it is possible to simultaneously reduce immune evasion mechanisms in the TME whilst increasing the target of the GD2-CAR T in the cancer cells.

### Example 13: Copper Chelation Therapy in Two Models of Mesothelioma

Both murine models (AB1-HA and AE17-OVA) were generated by exposure to asbestos. They display similar mutational burden and loss of key genes associated with human disease. The models are in two genetic backgrounds (BALB/c and C57BL/6), and respond to checkpoint therapy with α-CTLA4 targeting ICI and α-PDL1 targeting ICI treatment. These models were used to develop the current treatments in mesothelioma as part of the clinical trials. The AB1-HA model has higher cure rates with ICI treatment and is useful for determining increased response rate. The AE17-OVA model typically does not cure from ICI treatment alone, and is useful for finding adjunct therapies that can bolster response rates.

Animals received 2 weeks of daily doses of 800 mg per kg TETA orally, followed by alternative day dosing until the end of the experiment. ICI doses were administered according to the dosing schedule depicted in FIG. 42. No toxicity or loss of weight was observed in the mice.

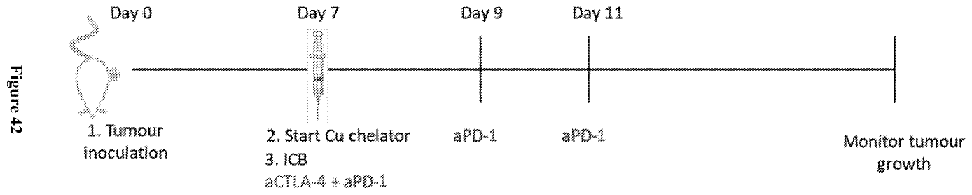

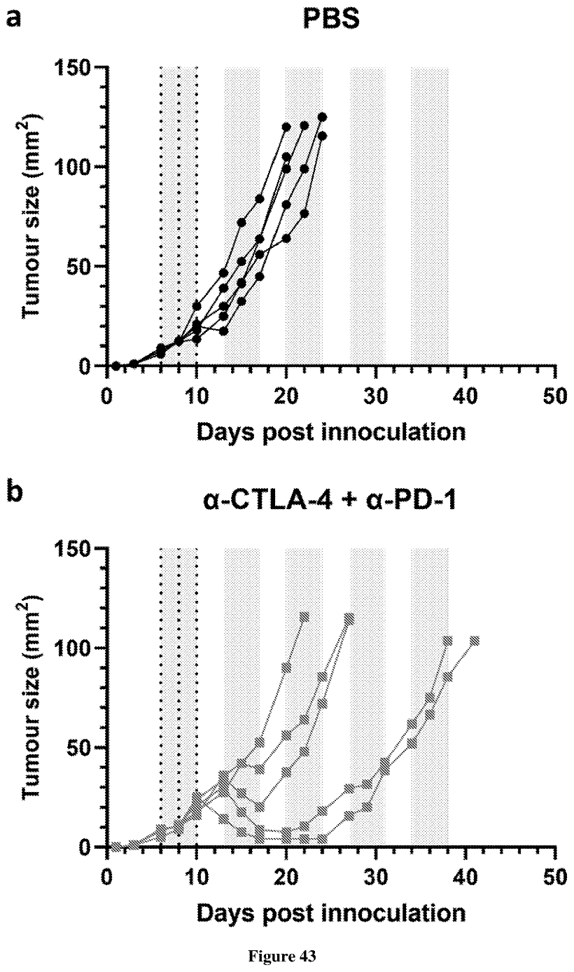

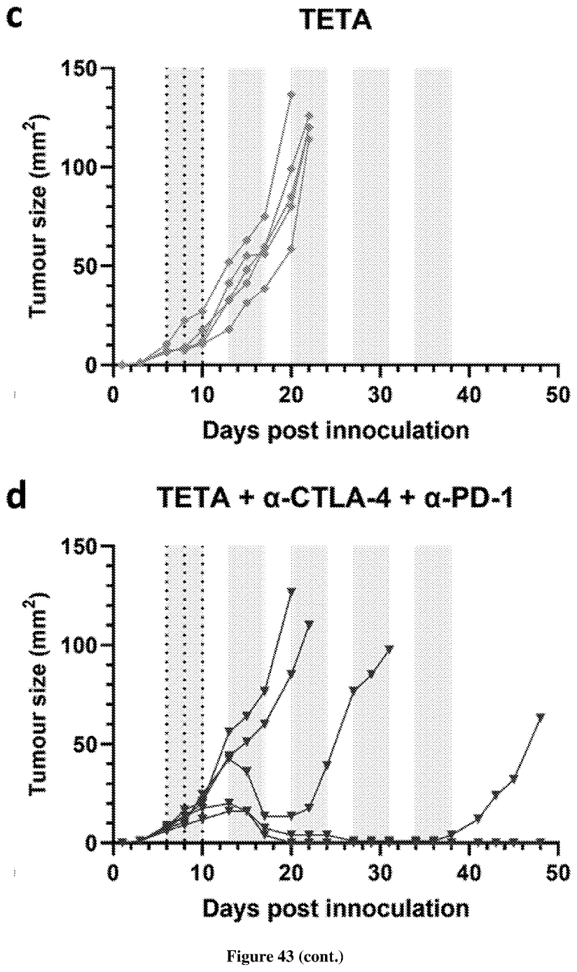

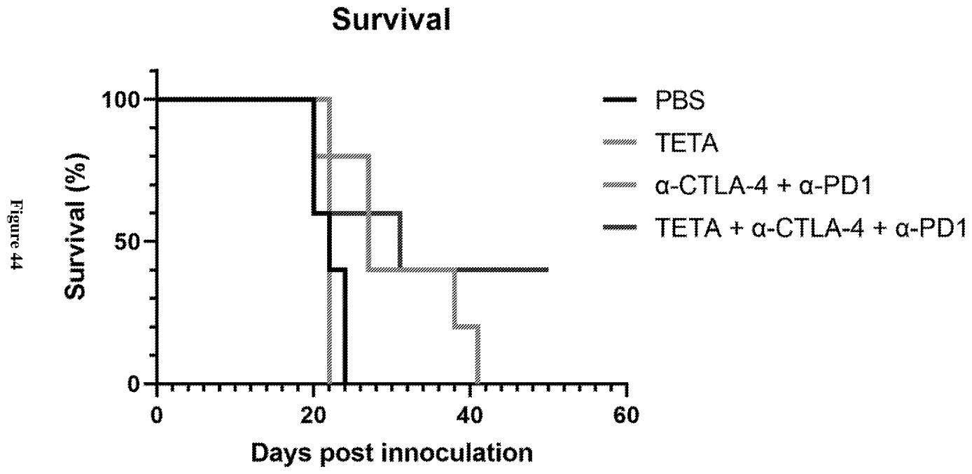

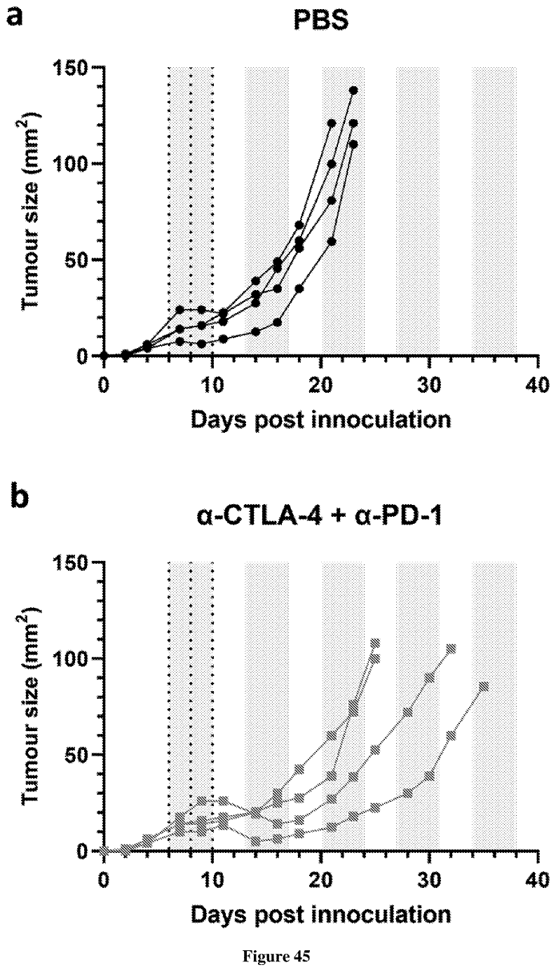

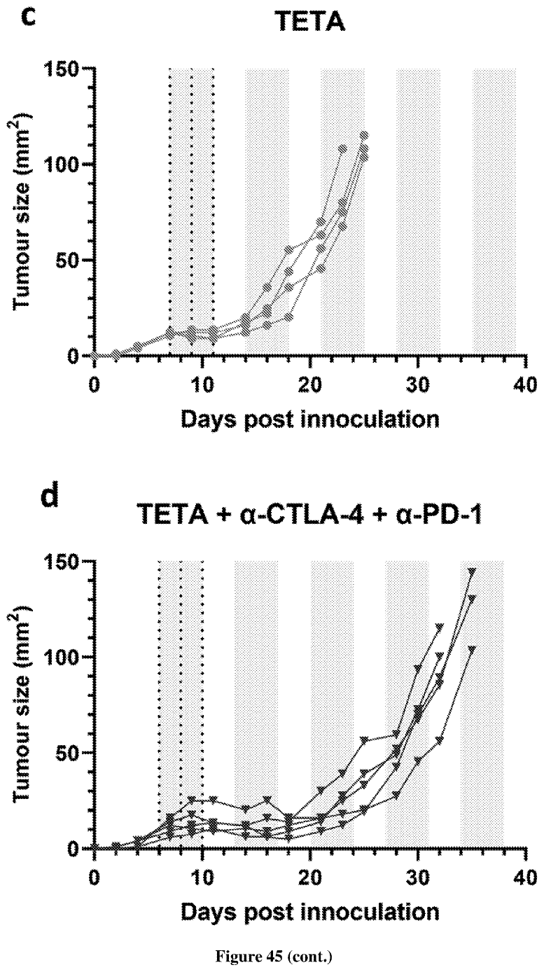

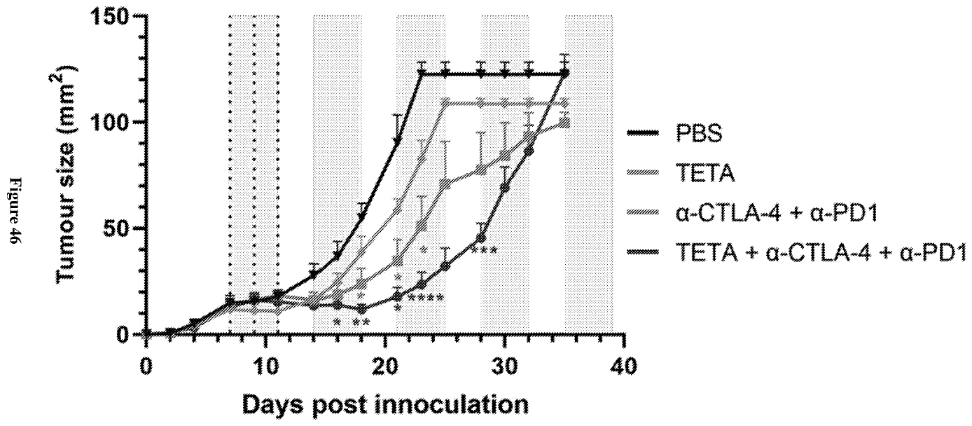

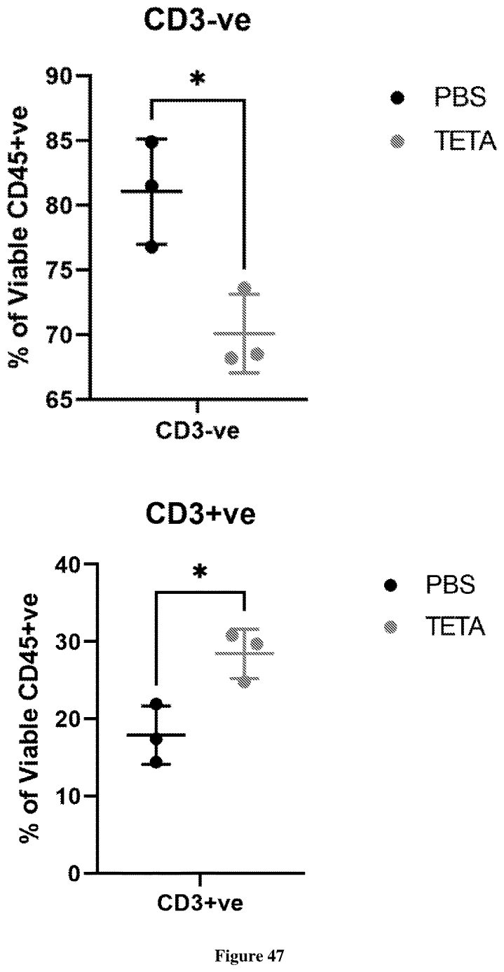

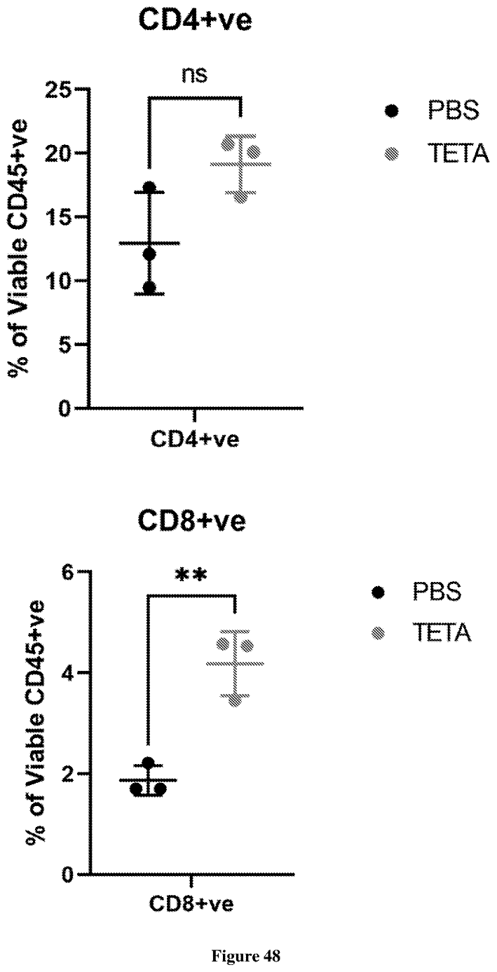

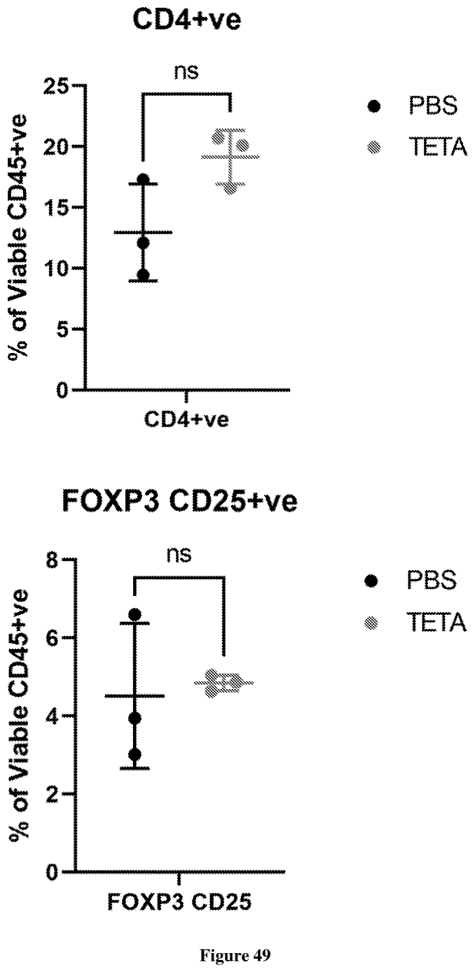

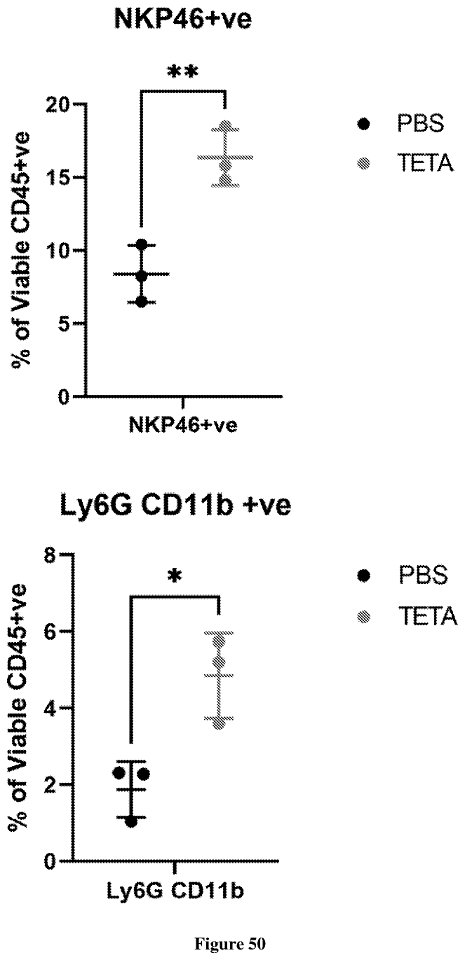

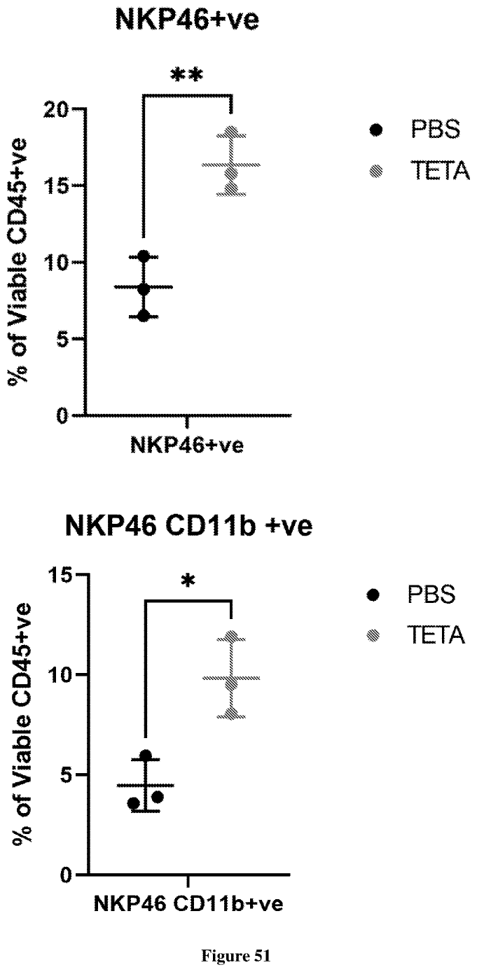

The tumor growth data (FIGS. 43 and 45) and survival data (FIGS. 44 and 46) for each model show an increased response rate and increase in overall survival using the combination treatment in the AB1-HA model of mesothelioma, and a significant decrease in tumour size with the combination treatment in the AE17-OVA model. Flow cytometry result (FIGS. 47-51) for mice treated for 1 week with 800 mg/kg TETA (1 week after inoculation) showed that CD8 and NK cells were significantly increased, and that there is a trend towards increased CD45 and neutrophils.

Based on the results shown in the examples above, a skilled person would reasonably expect that any copper chelator can be used in combination with any anticancer immunotherapy agent to treat any cancer type.

### METHODS

### In Vivo Studies

All experimental procedures were approved by the University of New South Wales Animal Care and Ethics Committee (ACEC) (Approval numbers ACEC 20/25B, 21/96B and 18/97B), according to the 1985 Animal Research Act (New South Wales, Australia) and the Australian Code of Practice for Care and Use of Animals for Scientific Purposes (2013). All animals were housed in a specific pathogen-free facility, with a maintained temperature of 22-24 °C on a 12 hr day/night cycle. Mice were housed in Ventirack cages (Tecniplast, Italy) and were provided food and water ad libitum and received environmental enrichment.

For in vivo treatment: the copper chelating agents tetraethylenepentamine pentahydrochloride (TEPA [C8H23N5·5HCl], Sigma, USA; #357683) and triethylenetetramine tetrahydrochloride (TETA [C6H18N4·4HCl], Sigma, USA; #161969) were freshly dissolved in saline vehicle and administered by oral gavage at 400 mg/kg. The anti-GD2 monoclonal antibody (clone 14G2a, #BE0318) and IgG isotype control (clone IgG2a, #BE0085) were obtained from BioXCell, USA and freshly diluted in medical-grade phosphate buffered saline and administered intraperitoneally in a 100 μg bolus.

The Th-MYCN model was kindly provided by Prof Michelle Haber (Children's Cancer Institute, Australia) and approved for use by the Institutional Biosafety Committee. Th-MYCN mice were maintained onsite, genotyped with only homozygous mice used experimentally. For tumor characterisation studies, male and female animals were recruited when a small tumor (3-4 mm³) was palpated and were treated for seven days with saline or TEPA (400 mg/kg) before tumor collection and sectioning for downstream applications. For immunotherapy combination studies, female and male mice were recruited as above and randomly assigned to the following treatment groups: Saline+saline vehicle; Saline+IgG; Saline+anti-GD2; TEPA+IgG; TEPA+anti-GD2. Regarding survival experiments, animals (n=5/group/sex) were weighed and palpated regularly for progression, regression or relapse and were sacrificed when tumor volume was determined ≥10 mm in diameter.

Female A/J mice were obtained from the Animal Resources Centre (Perth, Australia) and the NXS2 cell line was kindly provided by Prof Holger Lode (University of Greifswald, Germany). Animals aged 6-7 weeks were injected subcutaneously with 1.5×10⁶ NXS2 cells in a 1:1 mix of serum-free Dulbecco's Modified Eagle Media (DMEM; Gibco, USA, #11995065) and Matrigel (Corning, USA, #354234). Tumors engrafted for 7 days (reaching 50-100 mm³) before commencing treatment with saline or TEPA (400 mg/kg) for seven days. Mice were assigned to treatment groups to achieve approximately equal average initial tumor sizes to mitigate bias. Animals were weighed and tumor volumes measured twice weekly using digital callipers (calculated as 0.5×length×width2) or daily if over ≥800 mm³. Concerning immunotherapy combination studies, mice were assigned to the following treatment groups: Saline+saline vehicle; Saline+IgG; Saline+anti-GD2; TEPA+IgG; TEPA+anti-GD2. Animals were sacrificed once tumor volume reached ≥1000 mm³ in survival experiments.

### OPAL Multiplex Immunohistochemistry (IHC)

Tumor sections were formalin fixed and paraffin embedded (FFPE) by the Katharina Gaus Light Microscopy Facility (KGLMF) at the University of New South Wales. Tumors were sectioned at 4 μm and in preparation for staining, slides were baked for 1 hr at 58 °C before deparaffinization and rehydration using a Gemini AS Automated Slide Stainer (Epredia, USA). Chromogen-based IHC analysis was performed using the BOND-RX automated staining system (Leica Biosystems, USA). Spleens obtained from tumor-bearing Th-MYCN mice were used as a control for single antibody and OPAL multiplex optimisation. The following antibodies were all obtained from Abcam, UK: rabbit monoclonal NCR1 (clone EPR23097-35, #ab233558, 1:500, EDTA pH 8-9 antigen retrieval), rabbit monoclonal CD8a (clone EPR20305, #ab209775, 1:1000, EDTA pH 8-9 antigen retrieval), and rabbit monoclonal CD11b (clone EPR1344, #ab133357, 1:20000, citrate pH 6 antigen retrieval). Immunofluorescent signal was visualized using the OPAL 7-color Automation IHC kit (Akoya Biosciences, USA; #NEL871001KT) using TSA dyes 650, 570, and 520 respectively, and counterstained with spectral DAPI. Optimisation also included the sequence of antibodies which was determined to obtain the same dynamic ranges between each fluorophore to avoid signal “cross-talk” known as the umbrella effect. Labelled slides were imaged using the Vectra Polaris system (Akoya Biosciences, USA) using auto-exposure at 20× magnification. Whole slides were imaged using Phenochart software v1.1.0 (Akoya Biosciences, USA) via multispectral field scans. Acquired images were unmixed using Inform v2.5.1 (Akoya Biosciences, USA) and subsequently stitched together in HALO suite v3.6.4134.193 (Indica Labs, USA) to produce a whole-slide multispectral TIFF image. Nuclei segmentation was performed using HALO AI v3.6.4134 which leverages artificial intelligence and machine learning to train the software. Cell phenotyping analysis was performed using the HighPlex FL v4.2.5 module using defined thresholds for nuclear detection and minimum fluorescence intensity. The classifier was trained on all slides and necrotic areas were excluded prior to analysis. Whole tumor sections (necrotic areas excluded) were subjected to analysis and cells were classified as positive if fluorescence intensity exceeded a predefined threshold. Cell density was determined as the number of positive cells per 1000 defined nuclei per tumoral section.

### Single-Cell RNA Sequencing (scRNA-seq)

Fresh tumor sections were roughly minced and incubated in a tumor digestion mix consisting of DMEM supplemented with 25 μg/mL DNase I and 20 μg/mL Collagenase IV for 1 hr at 37 °C at 130 RPM. A single-cell dissociation was achieved by passing the mixture through a 70 μm MACS SmartStrainer (Miltenyi Biotec, Germany; #130-110-916). Cells were pelleted at 330×g for 5 min and resuspended in room temperature ACK Lysis Buffer to remove contaminating erythrocytes and neutralised with Stain Buffer (BD Biosciences, USA; #554656).

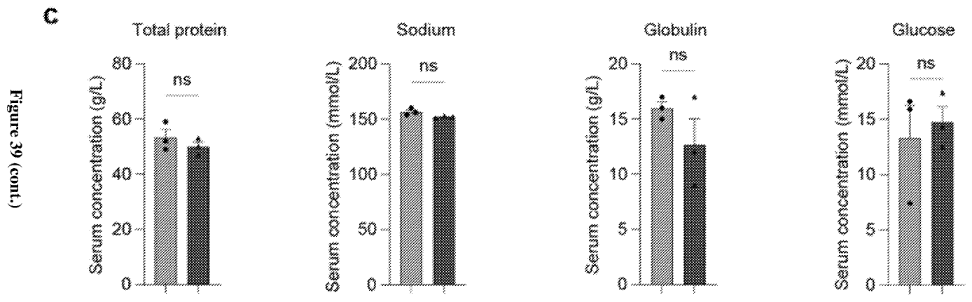

To reduce non-specific antibody staining of IgG receptors, 1×10⁶ cells were aliquoted and pre-incubated with Mouse BD Fc CD16/CD32 Block (BD Pharmingen, USA; #553142). Cells were incubated with CD3e-FITC (Thermo Fisher; #11-0031-82, clone 145-2C11, 1:100), NK1.1-PE (Thermo Fisher; #12-5941-82, clone PK136, 1:200) and CD11b-BV421 (BioLegend; #101251, clone M1/70, 1:200). Tumor cells were positively selected using a previously optimised gating strategy using absence of described markers. Approximately 50,000 single cells of each subset (CD3-NK1.1-CD11b-tumor cells, CD3+/NK1.1+ lymphocytes and CD11b+ myeloid cells) were sorted into a single tube containing foetal bovine serum (FBS; Gibco, USA, #10100-147) using the BD FACSAria III (BD Biosciences, USA). A representative gating strategy is presented in FIG. 40a.

The BD Rhapsody system (BD Biosciences, USA) was used to capture the transcriptomic data of approximately 25,000 total cells applied per cartridge (1× Control; 1× TEPA-treated). Whole transcriptome libraries were constructed following the BD Rhapsody single-cell whole-transcriptome amplification workflow according to the manufacturer's instructions. Libraries were quantified using a High Sensitivity DNA chip (Agilent, USA; #5067-4626) on a Bioanalyzer 2200 and the Qubit High Sensitivity double-stranded DNA Assay Kit (Thermo Fisher Scientific, USA; #Q32851). Resulting DNA libraries were sequenced on an Illumina NovaSeq 6000 S4 2×150 bp kit to yield an average of 80,000 reads per cell.

Raw sequencing data was converted into gene expression profiles for individual cells using the BD Rhapsody WTA Analysis Pipeline provided on the Seven Bridges Platform (Seven Bridges Genomics, USA). The pipeline involves the removal of low-quality reads, read alignment, gene expression quantification, and data normalisation. To reduce bias during dimensionality reduction, downstream analyses were conducted separately for tumor and immune cell compartments. To isolate the tumor compartment, cells expressing Mycn>0; Ptprc=0 were retained, excluding cells that express at least one lymphocyte-associated biomarker in the dataset (Cd3d, Cd3e, Cd3g, Cd8a, Cd8b1). This yielded a total of 13,560 cells across control and treated groups in the tumor compartment. To isolate the immune compartment, cells expressing Ptprc>0; Mycn=0 were retained, including the lymphocytes previously excluded from the tumor compartment. This yielded a total of 22,182 cells across control and treated groups in the immune compartment.

In the immune cell compartment, 12,127 cells passed filtering conditions with the following thresholds: 200<nFeature_RNA>5500 (number of transcripts); nCount_RNA<3000 (number of counts); percent.mt<25 (percentage of mitochondrial counts); and percent.ribo<20 (percentage of ribosomal counts). In the tumor cell compartment, 13,544 cells successfully passed the following filtering conditions: nFeature_RNA>200; percent.mt<25 and percent.ribo<15. Library-specific thresholds were manually assessed after exploring the empirical distribution of these variables in the 2D space. The CCA Integration method implemented in Seurat was used to perform integration-based anchoring to correct for batch effect. Raw counts were normalised through natural-log transformation.

Separate Uniform manifold approximation and projection (UMAP) plots were generated for the tumor and immune cell compartments. Principal Component Analysis (PCA) was used to reduce the dimensionality of the integrated dataset. For the immune cell compartment, 60 PCs were used to retain >70% of variability. Cells are then clustered and sub-clustered via the Louvain algorithm, with a resolution ranging between 0.2 and 0.3. For the tumor cell compartment, 80 PCs were used to retain >86% of variability. Cells were then clustered via the Louvain algorithm, with a resolution of 0.3.

Clustering annotation for the immune cell subsets was initially performed using the scType platform and subsequently curated manually based on the different gene markers identified by the MAST algorithm after batch correction. It was not possible to accurately assign a cell type to 2/15 identified immune clusters due to the minimal number of cells present (n<50) and were therefore excluded from analysis. Concerning the tumor cell compartment, it was not possible to accurately assign a cell type or phenotypic state to the 4 identified clusters after integration of control and treated samples however this observed variation corresponded to the difference between control and TEPA-treated groups. Final cell annotations were performed using relevant markers well-established in the literature and similarly confirmed using previous study annotations conducted by A/Prof Fabio Luciani.

Pathway enrichment analysis was performed to gain insights into the biological processes and pathways associated with each independent cell cluster. The analysis consisted of two main steps: Gene Set Enrichment Analysis (GSEA) using the fgsea function and Over-Representation Analysis (ORA) using the clusterProfiler package. The mouse hallmark gene sets from the Molecular Signatures Database (MsigDB) were used, along with selected pathways of interest obtained from Reactome, Gene Ontology (GO), and WikiPathways (WP) databases (GO:0048870, R-MMU-6798695, WP3941, WP4466, WP412). Additionally, custom-made signatures were included due to their unavailability in the public databases: copper-related genes, N1 anti-tumor phenotype and N2 pro-tumor phenotype.

For the GSEA, enrichment scores were calculated to evaluate the enrichment of the gene sets within the gene expression profiles of each cell type. The gene expression profiles were ranked based on the magnitude of change of genes significantly differentially expressed between control and treatment. To visualize the results, explanatory bar plots were generated per cell type, displaying the top significantly enriched pathways, each associated with a specific Negative Enrichment Score (NES) value. To further explore the enriched pathways, network plots were created to highlight the top 10 genes associated with each gene set. Non-relevant or less informative pathways were excluded from visualization to focus on the most relevant findings.

For the ORA analysis, the GO database was used to identify over-represented gene sets within each cell type. Significantly enriched GO terms associated with biological processes and molecular functions were identified using Fisher's exact test. To visualize the results of the ORA analysis, a selection of enriched pathways was made for each cell type. A network plot was generated, with each node representing a pathway and color-coded according to the corresponding cell type. Additionally, enriched genes within each pathway were identified and displayed within the network plot, allowing for a comprehensive view of the genes associated with each enriched pathway, per cell type.

Statistical tests used in the pathway enrichment analysis were implemented according to established methods. Multiple testing correction using the Benjamini-Hochberg procedure was applied to adjust p-values for multiple comparisons. The significance thresholds for determining enriched pathways were determined based on adjusted p-values and corresponds to p-adj<0.1.

Inference and analysis of cell-cell communication networks was performed using CellChat (v2) using the CellChatDB (v2) as the reference library which contains ~3,300 validated ligand-receptor interactions. The number of inferred signaling networks was narrowed down to 16 using a truncated mean of 25% i.e. for a given a certain cell group, the average gene expression of a certain ligand-receptor pair is set to zero if the percentage of expressed cells in that cell group is <25%.

### Tumoral Cytokine Profiling

Frozen tumor sections were homogenised in RIPA lysis buffer supplemented with 1× Protease and Phosphatase Inhibitors (Roche, USA; #04693159001) using the TissueRuptor II (Qiagen, Germany). To standardise cytokine concentrations, total extracted protein was calculated using the Pierce bicinchoninic acid (BCA) Protein Assay Kit (Thermo Fisher, USA; #23225) as per the manufacturer's instructions. Tumor cytokine levels were measured using the 36-Plex Mouse ProcartaPlex Panel 1A (Thermo Fisher, USA; #EPX360-26092-901) as per manufacturer's instructions. A Luminex MAGPIX System (Luminex Corporation, USA) was calibrated with MAGPIX Calibration and Performance Verification Kits (Millipore, USA) and data acquired using xPONENT software (Luminex Corporation, USA). Acquired data was analyzed using Multiplex Analyst software v5.1 (Merck, Germany) as the Median Fluorescent Intensity (MFI) with spline curve-fitting for calculating analyte concentrations in samples. Samples were diluted 1:10 with assay diluent prior to running and resulting concentrations normalized to extracted protein.

### MindRay Hematological Analysis

Peripheral blood was obtained from animals and collected in K2-EDTA tubes (Greiner, Germany; #450532) and analyzed immediately using the Mindray BC-5150 Auto Hematology Analyzer (Mindray, China) according to the manufacturer's instructions.

### Generation of the SK-N-BE(2)-C pCMV6-Ac-GFP Cell Line

The neuroblastoma cell line SK-N-BE(2)-C was obtained from the American Type Culture Collection with working stocks centrally managed by the Children's Cancer Institute Cell Bank. Both master and working stocks were validated using short tandem repeat profiling and routinely verified as Mycoplasma negative. Cells were cultured in Dulbecco's Modified Eagle Media (DMEM; Gibco, USA, #11995065) supplemented with 10% foetal bovine serum (FBS, Australian origin; Gibco, USA, #10100147). Cells were incubated under standard conditions (37 °C, 5% CO2, 95% humidity) and passaged routinely upon reaching a confluency of ~80%. The neuroblastoma cell line SK-N-BE(2)-C was stably transfected with plasmid pCMV6-Ac-GFP containing the transcript of human MT1X (NM_005952), with a c-terminal TurboGFP tag (tGFP) (Origene, USA; #RG207116). Cultures were kept under positive selection using Geneticin Selective Antibiotic (G418 [Thermo Fisher, USA; #10131035]) at 1 mg/mL. Cells were plated and treated with 6 mM TEPA for 24 hr and imaged using the IncuCyte Live-Cell Analysis system (Essen BioScience, USA). Images were taken in phase contrast and green fluorescence channels (auto-exposure) using a 10×60 objective.

### Staged Co-Culture Copper Transfer Assay

1.5 mL of media alone or containing 0.25×10⁶ SK-N-BE(2)-C cells were seeded in 2% FBS/DMEM before overnight treatment with 1 mM TEPA. The resulting conditioned media was collected immediately after neutrophils were ready for incubation. A portion of the conditioned media was collected as pre-incubation control, designated as: − neutrophil.

Peripheral blood from healthy donors was collected by venipuncture into K2-EDTA tubes (BD Biosciences, USA; #366643). Erythrocytes were sedimented using a 50% volume of Dextran solution (6% Dextran [Merck, USA; #09184-50G-F]; 0.9% NaCl in double-distilled water) for 30 min. A Percoll gradient was prepared using 90% Percoll solution (Percoll [Merck, USA; #P4937-100 ML] in 10×PBS (No Ca²⁺/Mg²⁺) to form bottom (~55% Percoll solution), middle (~68%) and top (~81%) layers in 1×PBS (No Ca²⁺/Mg²⁺).

The bottom layer was added to a fresh 15 mL Falcon tube followed by careful layering of the middle layer so as not to disturb the interface. The top layer of separated blood containing lymphocytes was collected, taking care to avoid contaminating erythrocytes, and moved into a fresh 15 mL Falcon tube. Tubes were centrifuged at 350×g for 20 min (speed 9 for both acceleration and brake) at 20 °C. The resulting plasma supernatant was decanted and pelleted lymphocytes were gently resuspended in the top Percoll layer which was then carefully layered on top of the previously prepared gradient. The resulting preparations were centrifuged at 700×g for 20 min (speed 0 for both acceleration and brake) at 20 °C. The resulting Percoll gradient yielded a top lymphocyte layer and a bottom neutrophil layer. The neutrophil layer was obtained and resuspended in 2% FBS/DMEM and centrifuged at 250×g for 6 min (speed 5 acceleration and speed 9 brake) at 20 °C. The supernatant was aspirated, and neutrophils were resuspended in 2% FBS/DMEM for counting.

0.25×10⁶ neutrophils were aliquoted into 1.5 mL Eppendorf tubes for each type of conditioned media and spun in a microfuge at 250×g for 6 min at 20 °C. The supernatant was aspirated, and pellets were resuspended in the respective conditioned media for 30 min at room temperature before centrifuging again at 1,000×g at 20 °C. Cell-free conditioned media (designated as: + neutrophil) was rapidly transferred to fresh 1.5 mL Eppendorf tubes for copper concentration analysis.

The concentration of copper in media samples was quantitatively determined using the QuantiChrom Copper Assay Kit (Universal Biologicals, UK; #DICU-250) according to the manufacturer's instructions. Fresh 2% FBS/DMEM was used as a blank to prepare the 300 μg/dL standard. Absorbance was determined using a Benchmark Plus Plate Reader with Microplate Manager v5.2.1 (Bio-Rad, USA) at a wavelength of 356 nm.

### Tissue Microarray Construction and Digital Spatial Profiling Hybridization

A tissue microarray (TMA) was prepared using 20 Th-MYCN tumor samples (10 control, 10 TEPA-treated) in duplicate, cored at 1 mm. The FFPE TMA block was sectioned at 4 μm and transferred to a Bond Plus slide (Leica Biosystems, USA; #S21.2113.A) and were processed by the Nanostring GeoMx DSP Technology Access Program. In brief, slides were hybridized with the GeoMx Mouse Whole Transcriptome Atlas (~18,000 targets) followed by immunofluorescent staining with pan-cytokeratin (PanCK; clone AE1/AE3, ThermoFisher; 53-9003-82) for identification of tumor cells, smooth muscle actin (SMA, clone 1A4, Abcam; #ab184675) for extracellular matrix, CD45 (clone D3F8Q, Cell Signaling Technology; #35154) for all hematopoietic cells and DNA GeoMx Nuclear Stain (Nanostring, #121303303) for cell nuclei. Post-staining slides were loaded onto the Nanostring GeoMx instrument and scanned.

For each tumor core, three geometric regions of interest (ROIs) were selected (ROIs=120) and were binarily defined as immune infiltrated or non-infiltrated, determined by the respective presence or absence of CD45 staining.

### Digital Spatial Profiling Data Processing

Segments and probes quality control was performed using the Bioconductor package GeomxTools. Of note, twenty ROIs containing necrotic sections were subsequently excluded. The Seurat package was used to perform the following downstream analyses. Principal Component Analysis (PCA) was used to reduce the dimensionality of the dataset, and 50 PCs were used to retain >85% of variability. Unsupervised clustering did not reveal any pattern of variation independent from treatment and infiltration. Pathway enrichment analysis was performed as described above in the single-cell sequencing dataset with three conditions were evaluated for differential gene expression and gene set enrichment within groups of ROIs: treatment vs control; infiltration vs non-infiltration (treated ROIs); treatment vs control (infiltrated ROIs). A gene set enrichment analysis (GSEA) was performed on the log 2-transformed and normalized gene expression matrix of infiltrated versus non-infiltrated TEPA-treated ROIs.

### Flow Cytometry Immunophenotyping

Tumor sections were dissociated into single cell suspensions as previously described above for flow cytometric staining. Cells were stained in fluorescence-activated cell sorting (FACS) Buffer (1× Phosphate buffered saline [PBS]/1% FBS/0.5 mM EDTA) using the following surface antibodies: CD45-BV510 (clone 30-F11, 1:250, BD Biosciences, #563891), CD11b-APC-ef780 (clone M1/70, 1:400, Thermo Fisher, #47-0112-82), Ly6G-streptavidin-BUV737 (clone 1A8, 1:300, BioLegend, #127604). After surface staining, BD Cytofix/Cytoperm Fixation/Permeabilization Kit (BD Biosciences, USA; #554714) was used according to the manufacturer's instructions, followed by intracellular staining with MMP9-APC (clone S51-82, 1:1250, StressMarq Biosciences, #SMC-396D-APC). Sample acquisition was performed using BD FACSAria III (BD Biosciences, USA) and analysed using FlowJo v10 (TreeStar, USA). A representative gating strategy is presented in FIG. 40b.

### VetScan Blood Chemistry Analysis

Peripheral blood was obtained from animals and collected in lithium heparin tubes (Greiner, Germany; #450536) and immediately analyzed using the VetScan VS2 Chemistry Analyzer (Zoetis, USA) using the Comprehensive Diagnostic Profile rotor (Zoetis, USA; #500-0038) according to the manufacturer's instructions.

### Statistical Analysis

All in vitro experiments were repeated at least three times with data presented as the means±standard error of the mean (SEM). Differences between two groups were determined with Mann-Whitney U tests (unpaired or paired where specified). Kaplan-Meier survival curves were analysed with two-tailed Mantel-Cox log-rank tests. With the exception of single-cell RNA sequencing data, all statistics were performed using GraphPad Prism v10 software (Dotmatics, UK). A p-value<0.05 was considered statistically significant for all experiments (*p≤0.005, **p≤0.01, ***p≤0.001, ns: not significant).

The examples disclosed herein show that copper chelators can be powerful modulators of the neuroblastoma microenvironment. They can slow MYCN-mediated tumour cell metabolism and progression, and can promote infiltration and activation of key subsets involved in ADCC. Significantly, they can enhance the efficacy of anti-GD2 immunotherapy. This effect could lead to improved patient response & outcomes in anti-GD2 immunotherapy, but also in other immunotherapies which would benefit from the action of copper inhibitors in inhibiting immunosuppression in the tumour microenvironment.

Other embodiments of the invention as described herein are defined in the following paragraphs:
1. A method of treating cancer, comprising administering a therapeutically effective amount of a copper chelator or a copper chelator inducing agent to a subject in need thereof in combination with a therapeutically effective amount of an anticancer immunotherapy agent.
2. Use of a copper chelator or a copper chelator inducing agent for the manufacture of a medicament for treating cancer in a subject in need thereof, wherein the copper chelator or copper chelator inducing agent is administered or is to be administered to the subject in combination with an anticancer immunotherapy agent.
3. Use of an anticancer immunotherapy agent for the manufacture of a medicament for treating cancer in a subject in need thereof, wherein the anticancer immunotherapy agent is administered or is to be administered to the subject in combination with a copper chelator or a copper chelator inducing agent.
4. Use of a copper chelator or a copper chelator inducing agent and an anticancer immunotherapy agent for the manufacture of a medicament for treating cancer in a subject in need thereof.
5. A copper chelator or a copper chelator inducing agent for use in the treatment of cancer in a subject in need thereof, wherein said copper chelator or copper chelator inducing agent is administered or is to be administered to the subject in combination with an anticancer immunotherapy agent.
6. An anticancer immunotherapy agent for use in the treatment of cancer in a subject in need thereof, wherein said anticancer immunotherapy agent is administered or is to be administered to the subject in combination with a copper chelator or a copper chelator inducing agent.
7. A combination comprising a copper chelator or a copper chelator inducing agent, and an anticancer immunotherapy agent for use in the treatment of cancer in a subject in need thereof.
8. The method, use, copper chelator or copper chelator inducing agent for use, anticancer immunotherapy agent for use, or combination, according to any one or more of the preceding paragraphs, wherein the copper chelator or copper chelator inducing agent and anticancer agent are administered, or are to be administered, separately to the subject.
9. The method, use, copper chelator or copper chelator inducing agent for use, anticancer immunotherapy agent for use, or combination, according to any one or more of the preceding paragraphs, wherein the copper chelator or copper chelator inducing agent and anticancer agent are administered, or are to be administered concurrently to the subject.
10. The method, use, copper chelator or copper chelator inducing agent for use, anticancer immunotherapy agent for use, or combination, according to any one or more of the preceding paragraphs, wherein the anticancer immunotherapy agent is selected from the group consisting of: antibodies, immune checkpoint inhibitors (e.g. α-CTLA4 targeting ICI and/or α-PDL1 targeting ICI), chimeric antigen receptor (CAR) T-cells, bispecific T-cell engagers (BiTEs), (CAR) NK-cells, adoptive immune cells and antibody-dependent immune cells, optionally the anticancer immunotherapy agent is an anti-GD2 immunotherapy agent, optionally the anticancer immunotherapy agent is a GD2-directed chimeric antigen receptor (CAR) T cell or an anti-GD2 antibody, optionally the anticancer immunotherapy agent is an anti-GD2 antibody, optionally the anticancer immunotherapy agent is selected from the group consisting of: atezolizumab (Tecentriq), avelumab (Bavencio), dostarlizumab (Jemperli), durvalumab (Imfinzi), ipilimumab (Yervoy), nivolumab (Opdivo), dinutuximab (Unituxin), dinutuximab beta (Qarziba), and pembrolizumab (Keytruda).
11. The method, use, copper chelator or copper chelator inducing agent for use, anticancer immunotherapy agent for use, or combination, according to any one or more of the preceding paragraphs, wherein the anticancer immunotherapy agent is an anti-GD2 immunotherapy agent, optionally a GD2-directed chimeric antigen receptor (CAR) T cell or an anti-GD2 antibody, optionally wherein the anticancer immunotherapy agent is an anti-GD2 antibody.
12. The method, use, copper chelator or copper chelator inducing agent for use, anticancer immunotherapy agent for use, or combination, according to any one or more of the preceding paragraphs, wherein the anticancer immunotherapy agent is selected from the group consisting of: atezolizumab (Tecentriq), avelumab (Bavencio), dostarlizumab (Jemperli), durvalumab (Imfinzi), ipilimumab (Yervoy), nivolumab (Opdivo), dinutuximab (Unituxin), dinutuximab beta (Qarziba), and pembrolizumab (Keytruda).
13. The method, use, copper chelator or copper chelator inducing agent for use, anticancer immunotherapy agent for use, or combination, according to any one or more of the preceding paragraphs, wherein the cancer is selected from the group consisting of lung cancer; breast cancer; colorectal cancer; anal cancer; pancreatic cancer; eye cancer; prostate cancer; ovarian carcinoma; liver and bile duct carcinoma; esophageal carcinoma; non-Hodgkin's lymphoma; bladder carcinoma; carcinoma of the uterus; glioma, glioblastoma, medulloblastoma, and other tumors of the brain; kidney cancer; myelofibrosis, cancer of the head and neck; cancer of the stomach; multiple myeloma; testicular cancer; germ cell tumor; neuroendocrine tumor; cervical cancer; oral cancer; carcinoids of the gastrointestinal tract, breast, and other organs; signet ring cell carcinoma; mesenchymal tumors including sarcomas, fibrosarcomas, haemangioma, angiomatosis, haemangiopericytoma, pseudoangiomatous stromal hyperplasia, myofibroblastoma, fibromatosis, inflammatory myofibroblastic tumour, lipoma, angiolipoma, granular cell tumour, neurofibroma, schwannoma, angiosarcoma, liposarcoma, rhabdomyosarcoma, osteosarcoma, leiomyoma or a leiomysarcoma; optionally neuroblastoma, breast cancer, glioblastoma, ovarian cancer, lung cancer, prostate cancer, stomach cancer, mesothelioma, liver cancer, cervical cancer, bladder cancer, thyroid cancer, pancreatic cancer, oral cancer, osteosarcoma, head and neck cancers, medulloblastoma, lower-grade gliomas, DMG (diffuse midline glioma), DIPG (diffuse intrinsic pontine glioma), and leukemia, optionally wherein the cancer is selected from neuroblastoma and mesothelioma; optionally, the cancer is selected from the group consisting of hepatocellular carcinoma, adenocarcinoma, breast cancer and prostate cancer; optionally the cancer is selected from the group consisting of neuroblastoma, breast cancer, glioblastoma, ovarian cancer, lung cancer, prostate cancer, stomach cancer, mesothelioma, liver cancer, cervical cancer, bladder cancer, thyroid cancer, pancreatic cancer, oral cancer, osteosarcoma, head and neck cancers, medulloblastoma, lower-grade gliomas, DMG (diffuse midline glioma), DIPG (diffuse intrinsic pontine glioma), and leukemia; optionally the cancer is breast cancer or neuroblastoma; optionally the cancer is selected from neuroblastoma and mesothelioma; optionally the cancer is neuroblastoma; optionally, the cancer is treatable by modulation of GD2, by for example, treatment with an anti-GD2 immunotherapy agent, such as an anti-GD2 antibody or a GD2-directed chimeric antigen receptor (CAR) T cell; optionally the cancer expresses GD2; optionally, the cancer is MYCN-mediated; optionally wherein the cancer is lung cancer it includes lung adenocarcinoma, squamous cell carcinoma, large cell carcinoma, bronchoalveolar carcinoma, non-small-cell carcinoma, small cell carcinoma and mesothelioma; optionally wherein the cancer is breast cancer it includes ductal carcinoma, lobular carcinoma, inflammatory breast cancer, clear cell carcinoma, and mucinous carcinoma; optionally wherein the cancer is colorectal cancer it includes colon cancer and rectal cancer; optionally wherein the cancer is pancreatic cancer it includes pancreatic adenocarcinoma, islet cell carcinoma and neuroendocrine tumors; optionally wherein the cancer is ovarian carcinoma it includes ovarian epithelial carcinoma or surface epithelial-stromal tumour including serous tumour, endometrioid tumor and mucinous cystadenocarcinoma, and sex-cord-stromal tumor; optionally wherein the cancer is liver and bile duct carcinoma it includes hepatocellular carcinoma, cholangiocarcinoma and hemangioma; optionally wherein the cancer is esophageal carcinoma it includes esophageal adenocarcinoma and squamous cell carcinoma; optionally wherein the cancer is carcinoma of the uterus it includes endometrial adenocarcinoma, uterine papillary serous carcinoma, uterine clear-cell carcinoma, uterine sarcomas and leiomyosarcomas and mixed Mullerian tumors; optionally wherein the cancer is kidney cancer it includes renal cell carcinoma, clear cell carcinoma and Wilm's tumor; optionally wherein the cancer is cancer of the head and neck it includes squamous cell carcinomas; optionally wherein the cancer is cancer of the stomach it includes stomach adenocarcinoma and gastrointestinal stromal tumor; optionally wherein the cancer is eye cancer it includes retinoblastoma and uveal melanoma.
14. The method, use, copper chelator or copper chelator inducing agent for use, anticancer immunotherapy agent for use, or combination, according to any one or more of the preceding paragraphs, wherein the copper chelator is selected from the group consisting of: ethylenediamine, polyethylenimine and derivatives thereof, tetrathiomolybdate; N,N′-bis(2-aminoethyl)ethane-1,2-diamine (trientine, triethylenetetramine, TETA); penicillamine; D-penicillamine; N′-(2-aminoethyl)-N-[2-(2-amino ethylamino) ethyl]ethane-1,2-diamine (tetraethylenepentamine, TEPA); 2-[2-[bis(carboxymethyl)amino]ethyl-(carboxymethyl)amino]acetic acid (EDTA); 2-[bis[2-[bis(carboxymethyl)amino]ethyl] amino]acetic acid (DTPA); N,N,N′,N′-tetrakis(2-pyridyl-methyl) ethylenediamine (TPEN); 2-[4,8,11-tris(carboxymethyl)-1,4,8,11-tetrazacyclotetradec-1-yl]acetic acid; 2-[4,7,10-tris(carboxymethyl)-1,4,7,10-tetrazacyclododec-1-yl]acetic acid (DOTA); (2S)-2-amino-3-methyl-3-sulfanylbutanoic acid (D-pen); bis(sulfanylidene)molybdenum sulfanide (TTM); 5-chloro-7-iodo-8-hydroxy-quinoline (Cliquinol); metformin; diethylcarbamodithioic acid (DDC); 2,9-dimethyl-1,10-phenanthroline (Neocuproine); 2,9-dimethyl-4,7-diphenyl-1,10-phenanthroline (Bathocuproine); 4-[2,9-dimethyl-7-(4-sulfophenyl)-1,10-phenanthrolin-4-yl]benzenesulfonic acid (BCS); and N,N′-bis(cyclohexylideneamino)oxamide (Cuprizone), optionally the copper chelator is selected from the group consisting of triethylenetetramine, tetraethylenepentamine, penicillamine, tetrathiomolibdic acid, and salts thereof, optionally the copper chelator is selected from the group consisting of tetraethylenepentamine, triethylenetetramine, and salts thereof; optionally the copper chelator is triethylenetetramine, or a salt thereof; optionally the copper chelator inducing agent is a zinc salt; optionally, the copper chelator is selected from the group consisting of triethylenetetramine, tetraethylenepentamine, penicillamine, tetrathiomolibdic acid, and salts thereof.
15. The method, use, copper chelator or copper chelator inducing agent for use, anticancer immunotherapy agent for use, or combination, according to any one or more of the preceding paragraphs, wherein the copper chelator is triethylenetetramine, or a salt thereof.
16. The method, use, copper chelator or copper chelator inducing agent for use, anticancer immunotherapy agent for use, or combination, according to any one or more of the preceding paragraphs, wherein the copper chelator inducing agent is a zinc salt.
17. The method, use, copper chelator or copper chelator inducing agent for use, anticancer immunotherapy agent for use, or combination, according to any one or more of the preceding paragraphs, wherein the copper chelator or copper chelator inducing agent and anticancer immunotherapy agent are administered or are to be administered in combination with a further active agent, optionally wherein the further active agent is an anticancer agent, optionally wherein the anticancer agent is selected from the group consisting of irinotecan (Camptosar) and temozolomide (Temodar).
18. A pharmaceutical composition comprising a copper chelator or a copper chelator inducing agent, and an anticancer immunotherapy agent.
19. The pharmaceutical composition according to any one or more of the preceding paragraphs, wherein the anticancer immunotherapy agent is selected from the group consisting of: antibodies, immune checkpoint inhibitors (e.g. α-CTLA4 targeting ICI and/or α-PDL1 targeting ICI), chimeric antigen receptor (CAR) T-cells, bispecific T-cell engagers (BiTEs), (CAR) NK-cells, adoptive immune cells and antibody-dependent immune cells, optionally the anticancer immunotherapy agent is an anti-GD2 immunotherapy agent, optionally the anticancer immunotherapy agent is a GD2-directed chimeric antigen receptor (CAR) T cell or an anti-GD2 antibody, optionally the anticancer immunotherapy agent is an anti-GD2 antibody, optionally the anticancer immunotherapy agent is selected from the group consisting of: atezolizumab (Tecentriq), avelumab (Bavencio), dostarlizumab (Jemperli), durvalumab (Imfinzi), ipilimumab (Yervoy), nivolumab (Opdivo), dinutuximab (Unituxin), dinutuximab beta (Qarziba), and pembrolizumab (Keytruda); optionally wherein the anticancer immunotherapy agent is an anti-GD2 immunotherapy agent, optionally a GD2-directed chimeric antigen receptor (CAR) T cell or an anti-GD2 antibody, optionally wherein the anticancer immunotherapy agent is an anti-GD2 antibody.
20. The pharmaceutical composition according to any one or more of the preceding paragraphs, wherein the anticancer immunotherapy agent is selected from the group consisting of: antibodies, immune checkpoint inhibitors (e.g. α-CTLA4 targeting ICI and/or α-PDL1 targeting ICI), chimeric antigen receptor (CAR) T-cells, bispecific T-cell engagers (BiTEs), (CAR) NK-cells, adoptive immune cells and antibody-dependent immune cells.
21. The pharmaceutical composition according to any one or more of the preceding paragraphs, wherein the copper chelator is selected from the group consisting of: ethylenediamine, polyethylenimine and derivatives thereof, tetrathiomolybdate; N,N′-bis(2-aminoethyl)ethane-1,2-diamine (trientine, triethylenetetramine, TETA); penicillamine; D-penicillamine; N′-(2-aminoethyl)-N-[2-(2-amino ethylamino) ethyl]ethane-1,2-diamine (tetraethylenepentamine, TEPA); 2-[2-[bis(carboxymethyl)amino]ethyl-(carboxymethyl)amino]acetic acid (EDTA); 2-[bis[2-[bis(carboxymethyl)amino]ethyl] amino]acetic acid (DTPA); N,N,N′,N′-tetrakis(2-pyridyl-methyl) ethylenediamine (TPEN); 2-[4,8,11-tris(carboxymethyl)-1,4,8,11-tetrazacyclotetradec-1-yl]acetic acid; 2-[4,7,10-tris(carboxymethyl)-1,4,7,10-tetrazacyclododec-1-yl]acetic acid (DOTA); (2S)-2-amino-3-methyl-3-sulfanylbutanoic acid (D-pen); bis(sulfanylidene)molybdenum sulfanide (TTM); 5-chloro-7-iodo-8-hydroxy-quinoline (Cliquinol); metformin; diethylcarbamodithioic acid (DDC); 2,9-dimethyl-1,10-phenanthroline (Neocuproine); 2,9-dimethyl-4,7-diphenyl-1,10-phenanthroline (Bathocuproine); 4-[2,9-dimethyl-7-(4-sulfophenyl)-1,10-phenanthrolin-4-yl]benzenesulfonic acid (BCS); and N,N′-bis(cyclohexylideneamino)oxamide (Cuprizone), optionally the copper chelator is selected from the group consisting of triethylenetetramine, tetraethylenepentamine, penicillamine, tetrathiomolibdic acid, and salts thereof, optionally the copper chelator is selected from the group consisting of tetraethylenepentamine, triethylenetetramine, and salts thereof; optionally the copper chelator is triethylenetetramine, or a salt thereof; optionally the copper chelator inducing agent is a zinc salt; optionally, the copper chelator is selected from the group consisting of triethylenetetramine, tetraethylenepentamine, penicillamine, tetrathiomolibdic acid, and salts thereof; optionally wherein the copper chelator is selected from the group consisting of triethylenetetramine, tetraethylenepentamine, penicillamine, tetrathiomolibdic acid, and salts thereof.
22. The pharmaceutical composition according to any one or more of the preceding paragraphs, wherein the copper chelator inducing agent is a zinc salt.

Although the invention has been described with reference to specific examples, it will be appreciated by those skilled in the art that the invention may be embodied in many other forms. In particular, features of any one of the various described examples may be provided in any combination in any of the other described examples. Various modifications and alterations to this invention will become apparent to those skilled in the art without departing from the scope and spirit of this invention. It should be understood that this invention is not intended to be unduly limited by the illustrative embodiments and examples set forth herein and that such examples and embodiments are presented by way of example only with the scope of the invention intended to be limited only by the claims set forth herein as follows.

1. A method of treating cancer, comprising administering a therapeutically effective amount of a copper chelator or a copper chelator inducing agent to a subject in need thereof in combination with a therapeutically effective amount of an anticancer immunotherapy agent.

2-6. (canceled)

7. A kit or combination comprising a copper chelator or a copper chelator inducing agent, and an anticancer immunotherapy agent for use in the treatment of cancer in a subject in need thereof.

8. The method of claim 1, wherein the copper chelator or copper chelator inducing agent and anticancer agent are administered separately to the subject.

9. The method of claim 1 wherein the copper chelator or copper chelator inducing agent and anticancer agent are administered concurrently to the subject.

10. The method of claim 1, wherein the anticancer immunotherapy agent is selected from the group consisting of: antibodies, immune checkpoint inhibitors, chimeric antigen receptor (CAR) T-cells, bispecific T-cell engagers (BiTEs), (CAR) NK-cells, adoptive immune cells and antibody-dependent immune cells.

11. The method of claim 1, wherein the anticancer immunotherapy agent is an anti-GD2 immunotherapy agent, optionally a GD2-directed chimeric antigen receptor (CAR) T cell or an anti-GD2 antibody, optionally wherein the anticancer immunotherapy agent is an anti-GD2 antibody.

12. The method of claim 1, wherein the anticancer immunotherapy agent is selected from the group consisting of: atezolizumab (Tecentriq), avelumab (Bavencio), dostarlizumab (Jemperli), durvalumab (Imfinzi), ipilimumab (Yervoy), nivolumab (Opdivo), dinutuximab (Unituxin), dinutuximab beta (Qarziba), and pembrolizumab (Keytruda).

13. The method of claim 1, wherein the cancer is selected from the group consisting of neuroblastoma, breast cancer, glioblastoma, ovarian cancer, lung cancer, prostate cancer, stomach cancer, mesothelioma, liver cancer, cervical cancer, bladder cancer, thyroid cancer, pancreatic cancer, oral cancer, osteosarcoma, head and neck cancers, medulloblastoma, lower-grade gliomas, DMG (diffuse midline glioma), DIPG (diffuse intrinsic pontine glioma), and leukaemia, optionally wherein the cancer is selected from neuroblastoma and mesothelioma.

14. The method of claim 1, wherein the copper chelator is selected from the group consisting of triethylenetetramine, tetraethylenepentamine, penicillamine, tetrathiomolybdic acid, and salts thereof, or wherein the copper chelator inducing agent is a zinc salt.

15. The method of claim 1, wherein the copper chelator is triethylenetetramine, or a salt thereof.

16. (canceled)

17. The method of claim 1, wherein the copper chelator or copper chelator inducing agent and anticancer immunotherapy agent are administered in combination with a further active agent, optionally wherein the further active agent is an anticancer agent, optionally wherein the anticancer agent is selected from the group consisting of irinotecan (Camptosar) and temozolomide (Temodar).

18. A pharmaceutical composition comprising a copper chelator or a copper chelator inducing agent, and an anticancer immunotherapy agent.

19. The pharmaceutical composition of claim 18, wherein the anticancer immunotherapy agent is an anti-GD2 immunotherapy agent, optionally a GD2-directed chimeric antigen receptor (CAR) T cell or an anti-GD2 antibody, optionally wherein the anticancer immunotherapy agent is an anti-GD2 antibody.

20. The pharmaceutical composition of claim 18, wherein the anticancer immunotherapy agent is selected from the group consisting of: antibodies, immune checkpoint inhibitors (e.g. α-CTLA4 targeting ICI and/or α-PDL1 targeting ICI), chimeric antigen receptor (CAR) T-cells, bispecific T-cell engagers (BiTEs), (CAR) NK-cells, adoptive immune cells and antibody-dependent immune cells.

21. The pharmaceutical composition of claim 18, wherein the copper chelator is selected from the group consisting of triethylenetetramine, tetraethylenepentamine, penicillamine, tetrathiomolybdic acid, and salts thereof; or wherein the copper chelator inducing agent is a zinc salt.

22. (canceled)

23. The kit or combination of claim 7, wherein the anticancer immunotherapy agent is an anti-GD2 immunotherapy agent, optionally a GD2-directed chimeric antigen receptor (CAR) T cell or an anti-GD2 antibody, optionally wherein the anticancer immunotherapy agent is an anti-GD2 antibody.

24. The kit or combination of claim 7, wherein the anticancer immunotherapy agent is selected from the group consisting of: antibodies, immune checkpoint inhibitors (e.g. α-CTLA4 targeting ICI and/or α-PDL1 targeting ICI), chimeric antigen receptor (CAR) T-cells, bispecific T-cell engagers (BiTEs), (CAR) NK-cells, adoptive immune cells and antibody-dependent immune cells.

25. The kit or combination of claim 7, wherein the anticancer immunotherapy agent is selected from the group consisting of: atezolizumab (Tecentriq), avelumab (Bavencio), dostarlizumab (Jemperli), durvalumab (Imfinzi), ipilimumab (Yervoy), nivolumab (Opdivo), dinutuximab (Unituxin), dinutuximab beta (Qarziba), and pembrolizumab (Keytruda).

26. The kit or combination of claim 7, wherein the copper chelator is selected from the group consisting of triethylenetetramine, tetraethylenepentamine, penicillamine, tetrathiomolybdic acid, and salts thereof, or wherein the copper chelator inducing agent is a zinc salt.

27. The kit or combination of claim 7, wherein the copper chelator is triethylenetetramine, or a salt thereof.
